(19) United States
(12) Patent Application Publication
(10) Pub. No.: US 2023/0391866 A1
(43) Pub. Date: Dec. 7, 2023

(54) POLYFUNCTIONAL ORTHOGONAL PROTEIN CHIMERAS

(71) Applicant: The Trustees of Columbia University in the City of New York, New York, NY (US)

(72) Inventors: Siddhartha MUKHERJEE, New York, NY (US); Abdullah Mahmood ALI, Tenafly, NJ (US); Alan BURKE, New York, NY (US); Florence BOROT, New York, NY (US)

(21) Appl. No.: 18/014,229

(22) PCT Filed: Jul. 6, 2021
(86) PCT No.: PCT/US2021/040538

§ 371 Date: Jan. 3, 2023

Related U.S. Application Data
(60) Provisional application No. 63/047,938, filed on Jul. 3, 2020.
(60) Provisional application No. 63/050,346, filed on Jul. 10, 2020.
(60) Provisional application No. 63/075,388, filed on Sep. 8, 2020.
(60) Provisional application No. 63/145,083, filed on Feb. 3, 2021.
(60) Provisional application No. 63/189,412, filed on May 17, 2021.

(51) Int. Cl. C07K 16/28; C07K 14/74; A61P 35/02

(52) U.S. Cl. CPC C07K 16/2803; C07K 16/2809; C07K 14/70539; A61P 35/02; C07K 16/283; C07K 2317/31; C07K 2317/622; C07K 2317/732; A61K 2039/505

(57) ABSTRACT

Disclosed herein are engineered heterodimer or heterotrimer proteins which use a non-naturally occurring polypeptide domain comprising 1-5 alpha helices connected by amino acid linkers and an IgG2 hinge domain either alone or in conjunction with an IgG2 Fc domain. The heterodimer and heterodimer proteins can further comprise an antigen-binding fragment that binds a lineage-specific cell-surface antigen, a polypeptide that binds to a molecule expressed on an immune cell (e.g., natural killer cell) and/or a polypeptide that binds to a molecule expressed on another type of immune cell (e.g., T cells). Also disclosed herein are nucleic acids encoding the proteins, vectors comprising the nucleic acids, compositions, and methods of treatment.

### CROSS-REFERENCE TO RELATED APPLICATIONS

The present application claims priority to U.S. Provisional Patent Application Nos. 63/047,938 filed Jul. 3, 2020, 63/050,346 filed Jul. 10, 2020, 63/075,388 filed Sep. 8, 2020, 63/145,083 filed Feb. 3, 2021, and 63/189,412 filed May 17, 2021, all of which are incorporated herein by reference in its entirety.

### SEQUENCE LISTING

The instant application contains a sequence listing which has been submitted electronically in ASCII format and is hereby incorporated by reference in its entirety.

### BACKGROUND

Presented on most acute myeloid leukemia (AML) leukemic cells, CD33 is an appealing target for immunotherapy, and represents a target population for which existing therapies are underperforming. Gemtuzumab ozogamicin, a drug fusing a monoclonal antibody to CD33 with a DNA-scission cytotoxic calicheamicin, was available for patients after first AML relapse between 2000 to 2010 before being pulled for increasing patient death rate without discernible benefit (Lowenburg et al. 2010). In 2017, it was reintroduced to market for primary CD33+ AML with modestly improved median survival in combination with cytosine arabinoside and daunorubicin, DNA intercalators that prevent DNA synthesis. As a monotherapy, it does not improve outcomes (Renneville et al. 2014). Bi-specific T-cell engagers (BiTE) which are currently in Phase I clinical trials, target CD33+ AML to CD3+ T-cells (Krupka et al. 2014). BiTEs, including those approved for B-cell malignancies such as blinatumomab which targets CD19+ B-cells for destruction via CD3+ T-cells, have such low bioavailability that they are injected continuously for weeks at a time, repeating for several months, prompting the need for alternative approaches to immune therapy (Nanbakhsh et al. 2014).

Many malignancies, including AML, are linked to decreased activity of the natural killer (NK) cytotoxic response. Expression of NK-activating ligands such as ULBP1 are positively correlated with survival but known to have depressed expression in AML even in conditions that would normally upregulate it (Elias et al. 2014). CD48, a ligand of the NK receptor 2B4, is downregulated on the surface of AML cells expressing fusion proteins such as AML1-ETO (Mastaglio et al. 2018). Conversely, NK-inhibiting ligands such as PD-L2 are upregulated (Dulphy et al. 2016). AML has in some cases been shown to dysregulate maturation of NK cells, as well as “distracting” NK cells with soluble or exosome-bound ligands of receptors such as NKG2D (Mundy-Bosse et al. 2014). Suppression of NK maturation, noted by low numbers of CD11b+CD27+NK cells, is more potent with increasing AML burden (Stringaris et al. 2014). The activating-inhibiting balance of NK cells—where cytotoxic function depends on expression of and engagement of inhibitory and activating receptors on NK cells—is frequently disturbed by AML, reducing surface expression of activating receptors and increasing expression of inhibiting receptors such as NKG2A (Orleans-Lindsay et al. 2001). In the case of exosomes or soluble ligands, NK cells struggle to find appropriate targets, revealing a therapeutic opportunity if a method can be developed that utilizes the NK cytotoxic response without relying on natural ligands on the surface of leukemic cells.

T-cells and NK cells alike have reduced function in AML contexts. Both primary blasts and AML cell lines like HL60 have demonstrated capacity to suppress the growth of T- and NK cells without affecting cytolytic activity or causing the death of these cells (LeDieu et al. 2009). A more comprehensive screen of patients suggested that T-cells in circulation greatly increase without an increase in activity, and others note increased proportion of T-regulatory cells in particular (Shenghui et al. 2011; Schnoffeil et al. 2015). Shifts toward memory T-cells, correlated with elevated PD-1 expression, have been demonstrated that once again do not suggest impairment in activity or in “exhaustion” of cytotoxic T-cells (Chamuleau et al. 2008). Breakdown of tryptophan and arginine via indoleamine 2,3-dioxygenase and arginase II, upregulated in some AMLs also contribute to poor proliferation and activation of T-cells, and may serve as a useful serum indicator of immune avoidance by myeloid malignancies (Thorsson et al. 2018). The inflammatory landscape is also implicated in AML's escape from T-cell activity, but the predictive and therapeutic potential of specific interactions has not been harnessed (Benci et al. 2016; Chen et al. 2019). Ultimately, the methods by which myeloid blasts avoid immune clearance-modulating immune sub-populations, inhibiting cytotoxicity, and masking themselves—are widely varied and difficult to predict.

Currently, new approaches are needed for diseases such as acute myeloid leukemia (AML) in which the outcomes, especially in older patients who are unable to receive intensive chemotherapy, the current standard of care, remains very poor, with a median survival of only 5 to 10 months (Dohner et al. 2015).

### SUMMARY

The present disclosure provides for engineered proteins or polyfunctional orthogonal protein chimeras.

Polyspecific or polyfunctional proteins are biomolecules that can simultaneously engage two or more different types of agents (proteins, DNA, RNA or cells) based on the specificity of interaction and affinity to each of these agents.

For example, bispecific or bifunctional antibodies genetically engineered from two different monoclonal antibodies, one with specificity to an immune cell (e.g., T or NK) and other with specificity towards a cancer cell, are being used to enhance tumor killing. Traditionally, these polyspecific proteins are made as a fusion protein but such fusion proteins may be rendered nonfunctional due to steric inhibition of engaging sites. Recent advances in synthetic biology enabled creation of synthetic polypeptides that allow for creation of orthogonal protein heterodimers formed via non-covalent interaction based on hydrogen bonding, similar to that observed between two anti-parallel strands of DNA.

Shown herein are improved polyspecific or polyfunctional or hetero or heterodimer or heterotrimer proteins which use a non-naturally occurring polypeptide domain comprising 1-5 alpha helices connected by amino acid linkers and an IgG2 hinge domain either alone or in conjunction with an IgG2 Fc domain. These polyfunctional proteins show improved properties over the previously described polyfunctional proteins. For one, the use of the IgG sequences allows for the formation of covalent disulfide bonds making the proteins more stable. These polyfunctional proteins have antibody-like properties and engage the body's complement system via the Fc domain. Furthermore, these polyfunctional proteins, like antibodies, are not easily destroyed in the body. Additionally, because these proteins are antibody-like, there are less immunogenic and have better pharmacokinetics than the standard, previously disclosed bispecific proteins.

While there are engineered proteins exemplified herein, the innovative use of the non-naturally occurring polypeptide domain comprising 1-5 alpha helices connected by amino acid linkers and an IgG2 hinge domain either alone or in conjunction with an IgG2 Fc domain can be expanded to construct engineered proteins with other specificities that also have superior binding and cytotoxic properties. These engineered proteins can comprise an antigen-binding fragment or other moiety which binds any lineage-specific cell surface antigen or other antigens expressed and/or over-expressed by cancer and tumor cells, and any polypeptide which binds to a molecule expressed on immune cells.

Exemplified herein is a heterodimer bispecific protein using a NKG2D binding domain of a protein ULBP1 to create a heterodimer with antigen recognition domain of monoclonal antibody that bind a CD33 protein (present on myeloid cells), to engage a NK cell with a myeloid cell. See FIGS. 1, 2 and 10.

Also, exemplified herein is a heterodimer bispecific protein using an antigen recognition domain of monoclonal antibodies that binds CD3 protein (present on T cells) and a CD33 protein (present on myeloid cells) to engage a T-cell with a myeloid cell. See FIGS. 1,2 and 10.

Also exemplified herein is a heterodimer bispecific protein using an antigen recognition domain of monoclonal antibodies that binds CD16 protein (present on NK cells) to create a heterodimer with antigen recognition domain of monoclonal antibody that bind a CD33 protein (present on myeloid cells), to engage a NK cell with a myeloid cell. See FIG. 23.

While the engineered proteins exemplified herein utilize an antigen-binding fragment or other moiety which binds a lineage-specific cell surface antigen (e.g., CD33), the disclosure includes engineered proteins comprising an antigen-binding fragment or other moiety which recognizes other antigens expressed and/or over-expressed by cancer and tumor cells. The disclosure also includes engineered proteins comprising polypeptides which bind to other molecules expressed on immune cells.

These exemplified heterodimer proteins comprise an IgG2 hinge domain. See FIGS. 1A and 23A.

Also shown herein are the exemplified heterodimer proteins comprising an IgG2 hinge domain and an IgG2 Fc domain (CH2 and CH3 domains). See FIGS. 1B and 23B.

Polyfunctional proteins can also be constructed comprising more than two polypeptides. These proteins can be formed by combining the monomer a of each heterodimer X, Y or Z with a linker and monomer b fused to a target binding domain. While this schematic shows a trispecific protein, tetraspecific proteins as well as engineered proteins comprising more than four can be constructed as shown in the schematic and methods herein. See FIG. 9.

The utility of these synthetic molecules includes therapeutic targeting, gene editing, diagnostics, pathway manipulation by activating and or deactivating two or more signals simultaneously.

The present disclosure provides for an engineered heterodimer protein. In some embodiments, a first engineered heterodimer protein comprises: a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and a second polypeptide comprising a polypeptide that binds a molecule expressed on natural killer (NK) cells, a non-naturally occurring polypeptide domain comprising 1-5 alpha helices connected by amino acid linkers, and a second covalent dimerization domain; and wherein the first and second polypeptides are covalently bonded through the covalent dimerization domain.

In some embodiments, a second engineered heterodimer protein comprises: a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and a second polypeptide comprising a polypeptide that binds a molecule expressed on T cells, a non-naturally occurring polypeptide domain comprising 1-5 alpha helices connected by amino acid linkers, and a second covalent dimerization domain; and wherein the first and second polypeptides are covalently bonded through the covalent dimerization domain.

In some embodiments, the non-naturally occurring polypeptide domain comprising 1-5 alpha helices connected by amino acid linkers in the first polypeptide and the second polypeptide are chosen from the group consisting of 6DMPb and 6DMPa.

In some embodiments, the first covalent dimerization domain and/or the second covalent dimerization domain comprise an IgG2 hinge domain. In some embodiments, the first covalent dimerization domain and/or the second covalent dimerization domain further comprise an IgG2 Fc domain.

In some embodiments, the lineage-specific cell-surface antigen may be CD33, CD19, or any of the lineage-specific cell-surface antigens described herein.

In some embodiments, the molecule expressed on the NK cells may be NKG2D, and the polypeptide that binds a molecule expressed on NK cells is ULBP1, ULBP2, ULBP3, ULBP4, ULBP5, ULBP6, MICA, MICB, or mutants or fragments thereof. In some embodiments, the polypeptide that binds a molecule expressed on NK cells is an ectodomain of ULBP1, ULBP2, ULBP3, ULBP4, ULBP5, ULBP6, MICA, or MICB.

In some embodiments, the molecule expressed on NK cells is CD16, and the polypeptide that binds a molecule expressed on NK cells is a monoclonal antibody of CD16.

In some embodiments, the molecule expressed on T cells is CD3, and the polypeptide that binds a molecule expressed on T cells is a monoclonal antibody of CD3.

In some embodiments, the antigen-binding fragment is a single-chain antibody fragment (scFv).

The present disclosure also provides for an engineered heterotrimer protein comprising three polypeptides, or an engineered protein comprising more than three polypeptides, comprising four polypeptides, comprising five polypeptides, comprising six polypeptides, or comprising more than six polypeptides, wherein the engineered hetero proteins comprise an additional polypeptide with binding domains to each of the other polypeptides in the engineered protein.

In some embodiments, the engineered heterotrimer protein comprises: a first polypeptide comprising a polypeptide that binds a molecule expressed on T cells, a non-naturally occurring polypeptide domain comprising 1-5 alpha helices connected by amino acid linkers (a1), and a first covalent dimerization domain; a second polypeptide comprising an antigen-binding fragment that binds a lineage-specific cell-surface antigen, a non-naturally occurring polypeptide domain comprising 1-5 alpha helices connected by amino acid linkers (b1), and a second covalent dimerization domain; a third polypeptide comprising a polypeptide that binds a molecule expressed on natural killer (NK) cells, a non-naturally occurring polypeptide domain comprising 1-5 alpha helices connected by amino acid linkers (c1), and a third covalent dimerization domain; and a fourth polypeptide comprising three non-naturally occurring polypeptide domains comprising 1-5 alpha helices connected by amino acid linkers, wherein each domain is the binding domain of a1, b1 and c1 (a2, b2 and c2), and a fourth, fifth and sixth covalent dimerization domain; and wherein the first and second and third and fourth polypeptides are covalently bonded through the covalent dimerization domain.

In some embodiments, the non-naturally occurring polypeptide domain comprising 1-5 alpha helices connected by amino acid linkers in the first polypeptide, the second polypeptide, the third polypeptide and the fourth polypeptide are chosen from the group consisting of 6DMPb and 6DMPa.

In some embodiments, the first covalent dimerization domain, the second covalent dimerization domain, the third covalent dimerization domain, the fourth covalent dimerization domain, the fifth covalent dimerization domain, and/or the sixth covalent dimerization domain comprise an IgG2 hinge domain. In some embodiments, one or more covalent dimerization domains further comprise an IgG2 Fc domain.

In some embodiments, the lineage-specific cell-surface antigen may be CD33, CD19, or any of the lineage-specific cell-surface antigens described herein.

In some embodiments, the molecule expressed on the NK cells may be NKG2D, and the polypeptide that binds a molecule expressed on NK cells is ULBP1, ULBP2, ULBP3, ULBP4, ULBP5, ULBP6, MICA, MICB, or mutants or fragments thereof. In some embodiments, the polypeptide that binds a molecule expressed on NK cells is an ectodomain of ULBP1, ULBP2, ULBP3, ULBP4, ULBP5, ULBP6, MICA, or MICB.

In some embodiments, the molecule expressed on the NK cells may be CD16 and the polypeptide that binds a molecule expressed on T cells is a monoclonal antibody of CD16.

In some embodiments, the molecule expressed on T cells is CD3, and the polypeptide that binds a molecule expressed on T cells is a monoclonal antibody of CD3.

In some embodiments, the third polypeptide is a chemokine or cytokine protein, which increases the immune response. The chemokine or cytokine protein includes but is not limited to CXCLs including CXCL14, GCSF, and interleukins, including IL2 and IL16.

In some embodiments, the antigen-binding fragment is a single-chain antibody fragment (scFv).

In certain embodiments, the first polypeptide in the first and second and third engineered heterodimer proteins comprises an amino acid at least 80% or at least 90% identical to SEQ ID NO: 1 (FIG. 3). In some embodiments, the first polypeptide in the engineered heterodimer proteins comprises an amino acid at least 80% or at least 90% identical to SEQ ID NO: 4 (FIG. 6).

In certain embodiments, the second polypeptide in the first engineered heterodimer comprises an amino acid at least 80% or at least 90% identical to SEQ ID NO: 2 (FIG. 4). In some embodiments, the second polypeptide in the first engineered heterodimer protein comprises an amino acid at least 80% or at least 90% identical to SEQ ID NO: 5 (FIG. 7).

In certain embodiments, the second polypeptide in the second engineered heterodimer protein comprises an amino acid at least 80% or at least 90% identical to SEQ ID NO: 3 (FIG. 5). In some embodiments, the second polypeptide in the second engineered heterodimer protein comprises an amino acid at least 80% or at least 90% identical to SEQ ID NO: 6 (FIG. 8).

In certain embodiments, the second polypeptide in the third engineered heterodimer protein comprises an amino acid at least 80% or at least 90% identical to SEQ ID NO: 26 (FIG. 23A). In some embodiments, the second polypeptide in the second engineered heterodimer protein comprises an amino acid at least 80% or at least 90% identical to SEQ ID NO: 27 (FIG. 23B).

The present disclosure provides for a composition comprising any of the engineered proteins, or a nucleic acid molecule encoding any of the engineered proteins.

The present disclosure also provides for a nucleic acid molecule encoding a first engineered heterodimer protein. The engineered dimer protein may comprise: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprising a polypeptide that binds a molecule expressed on natural killer (NK) cells, a non-naturally occurring polypeptide domain comprising 1-5 alpha helices connected by amino acid linkers, and a second covalent dimerization domain as described herein.

The present disclosure also provides for a nucleic acid molecule encoding a second engineered heterodimer protein. The engineered dimer protein may comprise: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprises a polypeptide that binds a molecule expressed on T cells, a non-naturally occurring polypeptide domain comprising 1-5 alpha helices connected by amino acid linkers, and a second covalent dimerization domain as described herein.

The present disclosure also provides for a nucleic acid molecule encoding a third engineered heterodimer protein. The engineered dimer protein may comprise: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprising a polypeptide that binds a molecule expressed on natural killer (NK) cells, a non-naturally occurring polypeptide domain comprising 1-5 alpha helices connected by amino acid linkers, and a second covalent dimerization domain as described herein.

The present disclosure also provides for a nucleic acid molecule encoding an engineered trimer protein. The engineered heterotrimer protein may comprise: (i) a first polypeptide comprises a polypeptide that binds a molecule expressed on T cells, a non-naturally occurring polypeptide domain comprising 1-5 alpha helices connected by amino acid linkers, and a second covalent dimerization domain an antigen-binding fragment that binds a lineage-specific cell-surface antigen; and (ii) a second polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (iii) a third polypeptide comprising a polypeptide that binds a molecule expressed on natural killer (NK) cells, a non-naturally occurring polypeptide domain comprising 1-5 alpha helices connected by amino acid linkers, and a second covalent dimerization domain; and (iv) a fourth polypeptide comprising three non-naturally occurring polypeptide domains comprising 1-5 alpha helices connected by amino acid linkers, wherein each domain is the binding domain of a1, b1 and c1 (a2, b2 and c2), and a fourth, fifth and sixth covalent dimerization domain, as described herein.

The present disclosure provides for a vector comprising any of the present nucleic acid molecules, or a composition comprising any of the present nucleic acid molecules.

The present disclosure provides for a cell comprising the present vectors or nucleic acid molecules.

The present disclosure provides for a composition comprising at least one vector encoding: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprising a polypeptide that binds a molecule expressed on natural killer (NK) cells, a non-naturally occurring polypeptide domain comprising 1-5 alpha helices connected by amino acid linkers, and a second covalent dimerization domain as described herein.

The present disclosure provides for a composition comprising at least one vector encoding: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprises a polypeptide that binds a molecule expressed on T cells, a non-naturally occurring polypeptide domain comprising 1-5 alpha helices connected by amino acid linkers, and a second covalent dimerization domain as described herein.

The present disclosure provides for a composition comprising at least one vector encoding: (i) a first polypeptide comprises a polypeptide that binds a molecule expressed on T cells, a non-naturally occurring polypeptide domain comprising 1-5 alpha helices connected by amino acid linkers, and a second covalent dimerization domain an antigen-binding fragment that binds a lineage-specific cell-surface antigen; and (ii) a second polypeptide comprising an antigen-binding fragment that binds a lineage-specific cell-surface antigen, a non-naturally occurring polypeptide domain comprising 1-5 alpha helices connected by amino acid linkers and a first covalent dimerization domain and (iii) a third polypeptide comprising a polypeptide that binds a molecule expressed on natural killer (NK) cells, a non-naturally occurring polypeptide domain comprising 1-5 alpha helices connected by amino acid linkers, and a second covalent dimerization domain; and (iv) a fourth polypeptide comprising three non-naturally occurring polypeptide domains comprising 1-5 alpha helices connected by amino acid linkers, wherein each domain is the binding domain of a1, b1 and c1 (a2, b2 and c2), and a fourth, fifth and sixth covalent dimerization domain, as described herein.

The present disclosure provides for a composition comprising any of the engineered proteins, any of the nucleic acid molecules, any of the present vectors, and/or any of the present cells.

Also encompassed by the present disclosure is a kit comprising any of the engineered proteins, any of the nucleic acid molecules, any of the present vectors, any of the present cells and/or any of the present compositions.

The present disclosure provides for a method of treating cancer in a subject, comprising administering to the subject an effective amount of any of the engineered proteins, any of the nucleic acid molecules, any of the present vectors, any of the present cells and/or any of the present compositions disclosed or described herein.

The present disclosure provides for a method of treating a hematopoietic malignancy in a subject, comprising administering to the subject an effective amount of any of the engineered proteins, any of the nucleic acid molecules, any of the present vectors, any of the present cells and/or any of the present compositions disclosed or described herein.

The hematopoietic malignancy may be a myeloid malignancy.

The hematopoietic malignancy may be Hodgkin's lymphoma, non-Hodgkin's lymphoma, leukemia, or multiple myeloma.

The hematopoietic malignancy may be acute myeloid leukemia, chronic myelogenous leukemia, acute lymphoblastic leukemia, or chronic lymphoblastic leukemia.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 are schematics of the various binding proteins. FIG. 1A is a schematic of the anti-CD33, anti-CD3, and NKG2D binding proteins with the IgG2 hinge domain only used in the engineered proteins. FIG. 1B is a schematic of the anti-CD33, anti-CD3, and NKG2D binding proteins with both IgG2 hinge and IgG2 Fc domains used in the engineered proteins.

FIG. 2 is a schematic of the bifunctional protein dimers engaging immune cells with cancer cells.

FIG. 3 is the amino acid sequence of the anti-CD33 construct with the IgG2 hinge domain only (SEQ ID NO: 1).

FIG. 4 is the amino acid sequence of the ULBP1 construct with the IgG2 hinge domain only (SEQ ID NO: 2).

FIG. 5 is the amino acid sequence of the anti-CD3 construct with the IgG2 hinge domain only (SEQ ID NO: 3).

FIG. 6 is the amino acid sequence of the anti-CD33 construct with both IgG2 hinge and IgG2 Fc domains (SEQ ID NO: 4).

FIG. 7 is the amino acid sequence of the ULBP1 construct with both IgG2 hinge and IgG2 Fc domains (SEQ ID NO: 5).

FIG. 8 is the amino acid sequence of the anti-CD3 construct with both IgG2 hinge and IgG2 Fc domains (SEQ ID NO: 6).

FIG. 9 is a schematic of an engineered heterotrimer protein.

FIG. 10 are schematics of the engineered heterodimer proteins with the IgG2-hinge and the 6DMPa/b heterodimerizing structure. FIG. 10A is an anti-CD33/anti-CD3 heterodimer protein. FIG. 10B is an anti-CD33/ULPB1 heterodimer protein.

FIG. 11 are plasmid maps of the expression vectors used in the studies. FIG. 11A is a map of the aCD33-6DMPa-Hinge expression vector. FIG. 11B is a map of the ULBP1-6DMPb-Hinge expression vector. FIG. 11C is a map of the aCD3-6DMPb expression vector.

FIG. 12 are immunoblots showing the expression and purification of the anti-CD33/ULBP engineered protein in 293T cells transfected with anti-CD33-ULBP1 chimeras 1 and 2 plasmids. FIG. 12A shows the expression in cells. FIG. 12B shows the expression and purification in supernatant.

FIG. 13 is an immunoblot showing the expression and purification of hinge-based constructs in CHO cells. The proteins are secreted into supernatant and can be 6×His-purified using cobalt-resin and elution with imidazole.

FIG. 14 shows FACS plots of HL-60 cells incubated with either anti-CD33 construct alone or with one of the engineered heterodimer proteins. FIG. 14A shows co-incubation with an anti-FLAG antibody. FIG. 14B shows co-incubation with an anti-MYC antibody.

FIG. 15 is a table summarizing binding experiments of the engineered heterodimer proteins to HL-60 cells.

FIG. 16 is a table summarizing binding experiments of the engineered heterodimer proteins to Jurkat cells.

FIG. 17 is a table summarizing binding experiments of the engineered heterodimer proteins to PMBCs.

FIG. 18 shows the result of a cytotoxicity assay using MOLM14 cells and the anti-CD33/anti-CD3 engineered heterodimer protein. FIG. 18A is a representative dot plot demonstrating flow cytometry gating scheme. Single dTomato+ (MOLM14) cells are gated, and % specific cells killed is assessed as total % of dTomato+ cells that are also DAPI+. FIG. 18B is a graph of the results of a cytotoxicity in MOLM14 cells using the anti-CD33/anti-CD3 engineered heterodimer protein.

FIG. 19 shows the result of a cytotoxicity assay using HL60 cells and the anti-CD33/anti-CD3 engineered heterodimer protein. FIG. 19A is a representative dot plot demonstrating flow cytometry gating scheme. Single CellTrace Violet+ (HL-60) cells are gated, and % specific cells killed is assessed as total % of CellTrace Violet+ cells that are also DAPI+. FIG. 19B is a graph showing the results of a cytotoxicity assay in HL-60 cells using the anti-CD33/anti-CD3 engineered heterodimer protein.

FIG. 20 is a graph showing further results of a cytotoxicity assay in HL-60 cells using the anti-CD33/anti-CD3 engineered heterodimer protein as compared to monomers.

FIG. 21 is a graph showing results of a dose dependent cytotoxicity assay using protein titration (i.e., increase in the anti-CD33/anti-CD3 engineered heterodimer protein) in HL-60 cells.

FIG. 22 is a graph showing results of a dose dependent cytotoxicity assay using effector titration (i.e., increase in the effector/target ratio) in HL-60 cells.

FIG. 23 are the amino acid sequences of the anti-CD16 construct. FIG. 23A is the amino acid sequence of the anti-CD16 construct with IgG2 hinge domain only (SEQ ID NO: 26). FIG. 23B is the amino acid sequence of the anti-CD16 construct with both IgG2 hinge and IgG2 Fc domains (SEQ ID NO: 27).

FIG. 24 shows the results of the in vivo anti-tumor activity of anti-CD33-anti-CD3 engineered heterodimer protein. FIG. 24A is a schematic of the experiment. FIG. 24B are images of bioluminescence imaging (BLI) used to monitor the growth of FFluc-dtomato transduced MOLM14. FIG. 24C is a graph of the quantification of BLI in mice treated with MOLM14 alone, unloaded T cells or anti-CD33-anti-CD3 engineered heterodimer protein loaded T cells. FIG. 24D is a Kaplan-Meier survival plot. Mice treated with anti-CD33-anti-CD3 engineered heterodimer protein loaded T cells have better survival than the 2 control groups (no treatment or unloaded T cells). Log-rank test *p<0.05.

### DETAILED DESCRIPTION

### Definitions

The term “engineered protein” or “engineered heterodimer protein” or “engineered heterotrimer protein” or “engineered hetero protein” or “polyfunctional protein” or “polyfunctional orthogonal protein chimera” or “protein chimera” or the like as used herein refers to a hybrid polypeptide which comprises protein domains from at least two different proteins. One domain may be located at the amino-terminal (N-terminal) portion of the fusion protein or at the carboxy-terminal (C-terminal) portion of the fusion protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include, but are not limited to, plasmids, phages, and viruses. Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA which codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct” or “gene construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, WI), pRSET or pREP plasmids (Invitrogen, San Diego, CA), or pMAL plasmids (New England Biolabs, Beverly, MA), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

The term “recombinant expression vector” means a genetically modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally occurring as a whole. Parts of the vectors can be naturally occurring. The non-naturally occurring recombinant expression vectors of the present disclosure can comprise any type of nucleotides, including, but not limited to DNA and RNA, which can be single-stranded or double-stranded, synthesized or obtained in part from natural sources, and which can contain natural, non-natural or altered nucleotides.

“Transfection,” “transformation,” or “transduction,” as used herein, refer to the introduction of one or more exogenous polynucleotides into a host cell by using physical or chemical methods.

“Antibody,” “fragment of an antibody,” “antibody fragment,” “functional fragment of an antibody,” or “antigen-binding portion” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide linked Fv, Fc, or variants and/or mixtures. The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

The present antibodies or antigen-binding portions can specifically bind with a dissociation constant (K_{D}) of less than about 10⁻⁷ M, less than about 10⁻⁸ M, less than about 10⁻⁹ M, less than about 10⁻¹⁰ M, less than about 10⁻¹¹ M, or less than about 10⁻¹² M. Affinities of the antibodies according to the present disclosure can be readily determined using conventional techniques (see, e.g., Scatchard et al., Ann. N. Y. Acad. Sci. (1949) 51:660; and U.S. Pat. Nos. 5,283,173, 5,468,614, or the equivalent).

The antigen recognition moiety of the engineered protein encoded by the nucleic acid sequence can contain any lineage antigen-specific, antigen-binding antibody fragment. The antibody fragment can comprise one or more CDRs, the variable region (or portions thereof), the constant region (or portions thereof), or combinations of any of the foregoing.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

The term “cell lineage” refers to cells with a common ancestry and developing from the same type of identifiable cell into specific identifiable/functioning cells. The cell lineages used herein include, but are not limited to, respiratory, prostatic, pancreatic, mammary, renal, intestinal, neural, skeletal, vascular, hepatic, hematopoietic, muscle or cardiac cell lineages.

The term “inhibition” when used in reference to gene expression or function of a lineage specific antigen refers to a decrease in the level of gene expression or function of the lineage specific antigen, where the inhibition is a result of interference with gene expression or function. The inhibition may be complete, in which case there is no detectable expression or function, or it may be partial. Partial inhibition can range from near complete inhibition to a near absence of inhibition.

The terms “treat”, “treatment”, and the like refer to a means to slow down, relieve, ameliorate or alleviate at least one of the symptoms of the disease, or reverse the disease after its onset.

“Treating” or “treatment” of a state, disorder or condition includes:
(1) preventing or delaying the appearance of clinical symptoms of the state, disorder, or condition developing in a person who may be afflicted with or predisposed to the state, disorder or condition but does not yet experience or display clinical symptoms of the state, disorder or condition; or
(2) inhibiting the state, disorder or condition, i.e., arresting, reducing or delaying the development of the disease or a relapse thereof (in case of maintenance treatment) or at least one clinical symptom, sign, or test, thereof; or
(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

The terms “prevent”, “prevention”, and the like refer to acting prior to overt disease onset, to prevent the disease from developing or minimize the extent of the disease or slow its course of development.

An “immune response” refers to the development in the host of a cellular and/or antibody-mediated immune response to a composition or vaccine of interest. Such a response usually consists of the subject producing antibodies, B cells, helper T cells, suppressor T cells, regulatory T cells, and/or cytotoxic T cells directed specifically to an antigen or antigens included in the composition or vaccine of interest.

A “therapeutically effective amount” or “effective amount” means the amount of a compound or agent that, when administered to an animal for treating a state, disorder or condition, is sufficient to affect such treatment. The “therapeutically effective amount” will vary depending on the compound, the disease and its severity and the age, weight, physical condition and responsiveness of the animal to be treated.

The compositions disclosed herein may include a “therapeutically effective amount” or a “prophylactically effective amount” of a compound described herein. A “therapeutically effective amount” refers to an amount effective, at dosages and for periods of time necessary, to achieve the desired therapeutic result. A therapeutically effective amount of an antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

While it is possible to use a composition provided by the present disclosure for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present disclosure provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the disclosure can be formulated for administration in any convenient way for use in human or veterinary medicine. The invention therefore includes within its scope pharmaceutical compositions comprising a product of the present invention that is adapted for use in human or veterinary medicine.

The term “pharmaceutical composition,” as used herein, refers to a composition that can be administrated to a subject in the context of treatment and/or prevention of a disease or disorder. In some embodiments, a pharmaceutical composition comprises an active ingredient, e.g., the present fusion polypeptide, nucleic acid molecule, vector, agent, etc., and optionally a pharmaceutically acceptable excipient, diluent and/or carrier.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985); Transcription and Translation (B. D. Hames & S. J. Higgins, eds. (1984»; Animal Cell Culture (R. I. Freshney, ed. (1986»; Immobilized Cells and Enzymes (lRL Press, (1986).

The present disclosure provides for agents comprising an antigen-binding fragment that binds a lineage-specific cell-surface antigen (e.g., CD33) which can cause cell death of the cells expressing the lineage-specific cell-surface antigen. Immunotherapies involving the combination of an antigen-binding fragment that binds a lineage-specific cell-surface antigen (e.g., CD33), and a polypeptide that binds a molecule expressed on an immune cell such as a natural killer (NK) cell and/or a T cell, would provide an efficacious method of treatment for hematopoietic malignancies.

The present disclosure provides for a first and a third engineered heterodimer protein, comprising (or consisting essentially of, or consisting of): (i) an antigen-binding fragment that binds a lineage-specific cell-surface antigen (e.g., CD33); and (ii) a polypeptide that binds a molecule expressed on immune cells. In some embodiments, the immune cells are natural killer (NK) cells. In some embodiments, the molecule is ULBP. In some embodiments, the molecule is CD16.

The present disclosure provides for a second engineered heterodimer protein, comprising (or consisting essentially of, or consisting of): (i) an antigen-binding fragment that binds a lineage-specific cell-surface antigen (e.g., CD33); and (ii) a polypeptide that binds a molecule expressed on T cells. In some embodiments, the molecule is CD3.

The present disclosure further provides for an engineered heterotrimer protein, comprising (or consisting essentially of, or consisting of): (i) an antigen-binding fragment that binds a lineage-specific cell-surface antigen (e.g., CD33); and (ii) a polypeptide that binds to immune cells (e.g., NK cells); and (iii) a polypeptide that binds a molecule expressed on T cells (e.g., CD3).

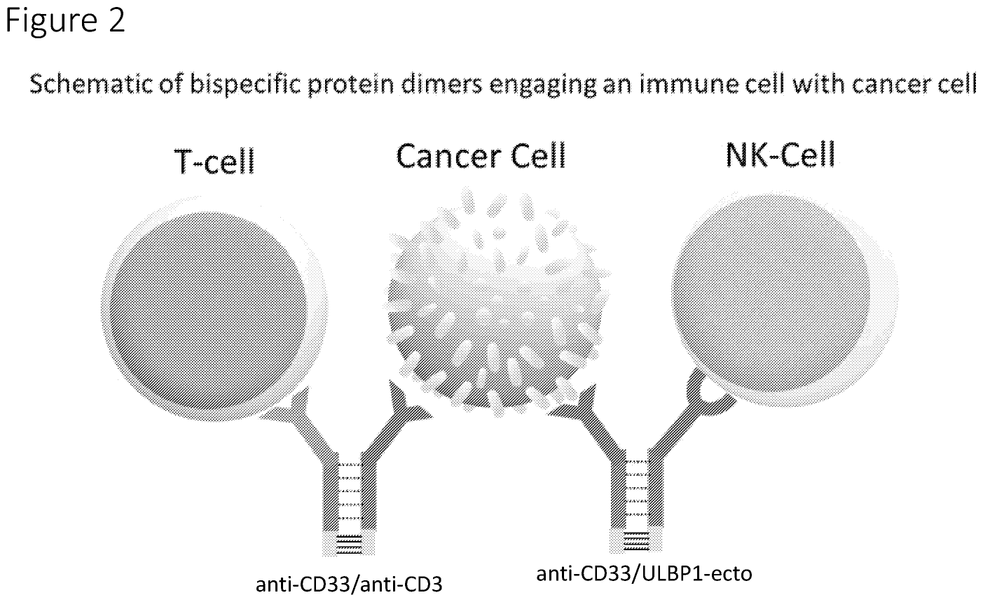

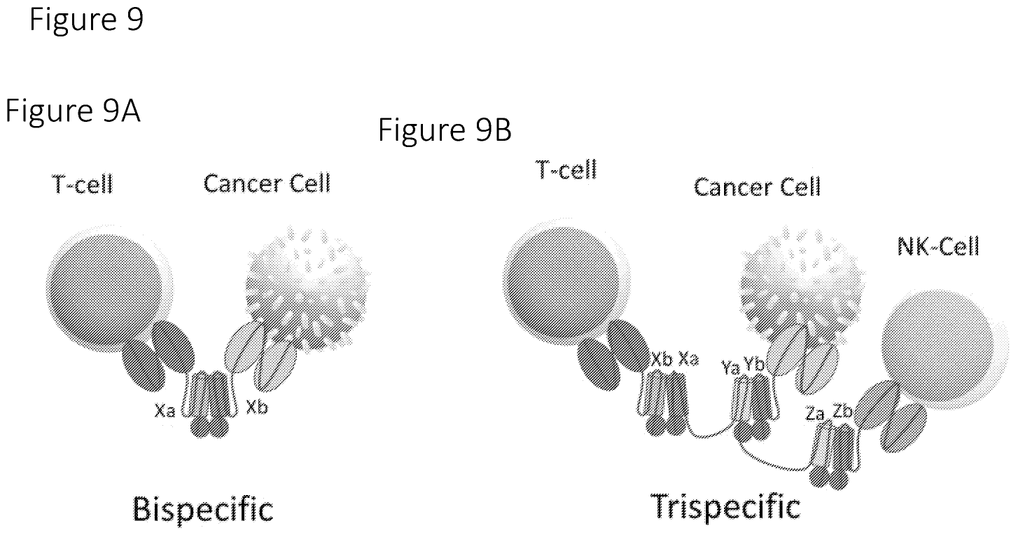

See FIGS. 2, 9, and 10.

The present compositions and methods may help activate NKG2D-bearing immune effector cells, such as natural killer (NK) cells and/or CD8+ T cells. The present compositions and methods may enhance or prompt a cellular immune response against diseased cells (such as tumor cells) that may induce cytotoxicity (e.g., culminate in the death of the diseased cells such as tumor cells). The present compositions and methods may enhance a subject's immune response, including, but not limited to one or more of the following: upregulation of natural killer (NK) cell; upregulation of T cell (e.g., gamma delta T cell, alpha beta T cell) function; upregulation of natural killer T (NKT) cell function; and upregulation of B cell function. In some embodiments, upregulation of one or more of NK cell, T cell, natural killer T (NKT) cell, and B cell function includes enhancement and/or endowment of activity capable of inhibiting or decreasing cancer progression.

In some embodiments, inhibiting cancer progression may be accomplished by cytolysis of tumor cells, e.g., by direct induction of tumor cell apoptosis, induction of tumor cell cytolysis through stimulation of intrinsic host antitumor responses, induction of tumor cell apoptosis through stimulation of intrinsic host antitumor responses, inhibition of tumor cell metastasis, inhibition of tumor cell proliferation, and induction of senescence in the tumor cell.

The present disclosure also provides one or more nucleic acid (polynucleotide) molecules encoding the present engineered proteins, agents or compositions.

Other aspects of the present disclosure provide vectors comprising any of the nucleic acid (or polynucleotide) molecules provided herein. Also, within the scope of the present disclosure are polynucleotides encoded by the nucleic acids described herein and cells expressing such polynucleotides.

In some embodiments, the cells can be obtained from a patient having a hematopoietic malignancy. In some embodiments, the cell is a hematopoietic cell, such as a hematopoietic stem cell (e.g., CD34⁺). In some embodiments, cells are provided, e.g., for recombinant expression and purification of the engineered proteins provided herein. The cells include any cell suitable for recombinant protein expression, for example, cells comprising a genetic construct or vector expressing or capable of expressing an engineered proteins (e.g., cells that have been transformed or transfected with one or more vectors described herein, or cells having genomic modifications, for example, those that express a protein provided herein). Methods for transforming cells, genetically modifying cells, and expressing genes and proteins in such cells are well known in the art, and include those provided by, for example, Green and Sambrook, Molecular Cloning: A Laboratory Manual (4th ed., Cold Spring Harbor Laboratory Press, Cold Spring Harbor, N.Y. (2012)) and Friedman and Rossi, Gene Transfer: Delivery and Expression of DNA and RNA, A Laboratory Manual (1st ed., Cold Spring Harbor Laboratory Press, Cold Spring Harbor, N.Y. (2006)).

Further, the present disclosure provides pharmaceutical compositions comprising the present agents, polypeptides, nucleic acid (or polynucleotide) molecules, vectors, cells, and/or compositions.

The present disclosure also provides for a method of treating a hematopoietic malignancy. The method may comprise administering to a subject in need thereof an effective amount of any of the disclosed engineered proteins or a polynucleotide encoding the engineered proteins. The method may comprise administering to a subject in need thereof an effective amount of the present agents or composition, or a polynucleotide encoding the agents (e.g., a combination of polypeptides) or compositions.

Another aspect of the present disclosure provides a method for treating a hematopoietic malignancy (or a hematological neoplasm), the method comprising administering to a subject in need thereof an effective amount of any of the disclosed engineered proteins or a polynucleotide encoding the engineered proteins. The method may comprise administering to a subject in need thereof an effective amount of the present agents or composition, or a polynucleotide encoding the agents (e.g., a combination of polypeptides) or compositions.

The present disclosure also relates to methods of using the engineered proteins to treat hematopoietic malignancies such as myeloid malignancies.

Also, within the scope of the present disclosure are kits comprising the present agents, polypeptides, nucleic acid (or polynucleotide) molecules, vectors, cells, and/or compositions.

### Engineered Hetero Proteins

The current disclosure provides for engineered hetero proteins.

In one embodiment, the present disclosure provides for a first and third engineered heterodimer protein, comprising (or consisting essentially of, or consisting of): (i) an antigen-binding fragment that binds a lineage-specific cell-surface antigen (e.g., CD33); and (ii) a polypeptide that binds a molecule expressed on immune cells. In some embodiments, the immune cells are natural killer (NK) cells. In some embodiments, the molecule is ULBP. In some embodiments, the molecule is CD16.

In a further embodiment, the present disclosure provides for a second engineered heterodimer protein, comprising (or consisting essentially of, or consisting of): (i) an antigen-binding fragment that binds a lineage-specific cell-surface antigen (e.g., CD33); and (ii) a polypeptide that binds a molecule expressed on T cells. In some embodiments, the molecule is CD3.

In yet a further embodiment, the present disclosure provides for an engineered heterotrimer protein, comprising (or consisting essentially of, or consisting of): (i) an antigen-binding fragment that binds a lineage-specific cell-surface antigen (e.g., CD33); and (ii) a polypeptide that binds to immune cells (e.g., NK cells); and (iii) a polypeptide that binds a molecule expressed on T cells (e.g., CD3).

In some embodiments, the third polypeptide is a chemokine or cytokine protein, which increases the immune response. The chemokine or cytokine protein includes but is not limited to CXCLs including CXCL14, GCSF, and interleukins, including IL2 and IL16.

In some embodiments, the antigen-binding fragment binds a lineage-specific cell-surface antigen that is a type 2 lineage-specific cell-surface antigen (e.g., CD33). In some embodiments, the antigen-binding fragment binds a lineage-specific cell-surface antigen that is a type 1 lineage-specific cell-surface antigen (e.g., CD19). In some embodiments, the antigen-binding fragment binds an antigen expressed or over-expressed by cancer and/or tumor cells.

The polypeptide that binds a molecule expressed on natural killer (NK) cells may be a fragment of fragment of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18, homologs thereof, mutants thereof, or fragments thereof. The polypeptide that binds a molecule expressed on natural killer (NK) cells may be an ectodomain of ULBP1, ULBP2, ULBP3, ULBP4, ULBP5, ULBP6, MICA, MICB, Raet1a, Raet1b, Raet1c, Raet1d, Raet1e, H60b, H60c, HCMV UL18, homologs thereof, mutants thereof, or fragments thereof.

In certain embodiments, the fragment of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18 comprises an ectodomain of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18. In certain embodiments, the fragment of ULBP1, ULBP2, ULBP3, ULBP4, ULBP5, ULBP6, MICA, MICB, Raet1a, Raet1b, Raet1c, Raet1d, Raet1e, H60b, H60c, or HCMV UL18 comprises an ectodomain of ULBP1, ULBP2, ULBP3, ULBP4, ULBP5, ULBP6, MICA, MICB, Raet1a, Raet1b, Raet1c, Raet1d, Raet1e, H60b, H60c, HCMV UL18, homologs thereof, mutants thereof, or fragments thereof.

In some embodiments, the polypeptide binds a molecule expressed on NK cells is an antibody of a cell surface marker of NK cells. In some embodiments, the cell surface marker is CD16. In some embodiments, the antibody is a monoclonal antibody.

In certain embodiments, the polypeptide that binds a molecule expressed on T cells is an antibody of a cell surface marker of T cells. In some embodiments, the cell surface marker is CD3. In some embodiments, the antibody is a monoclonal antibody.

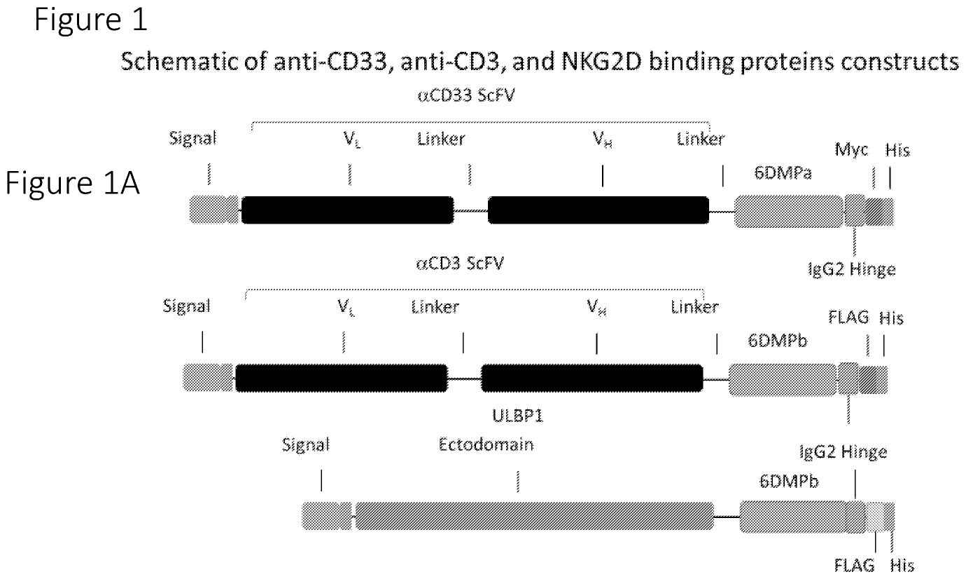

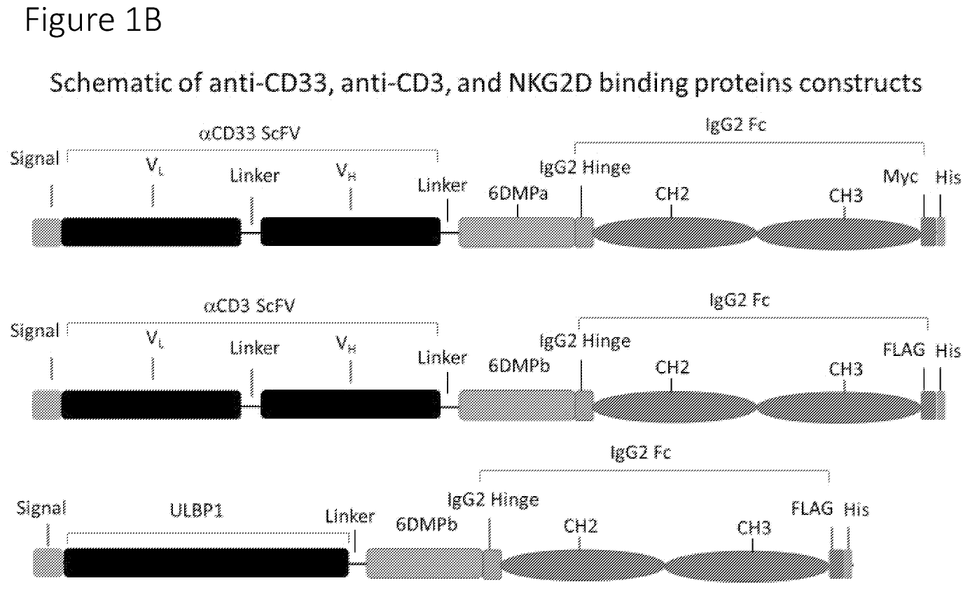

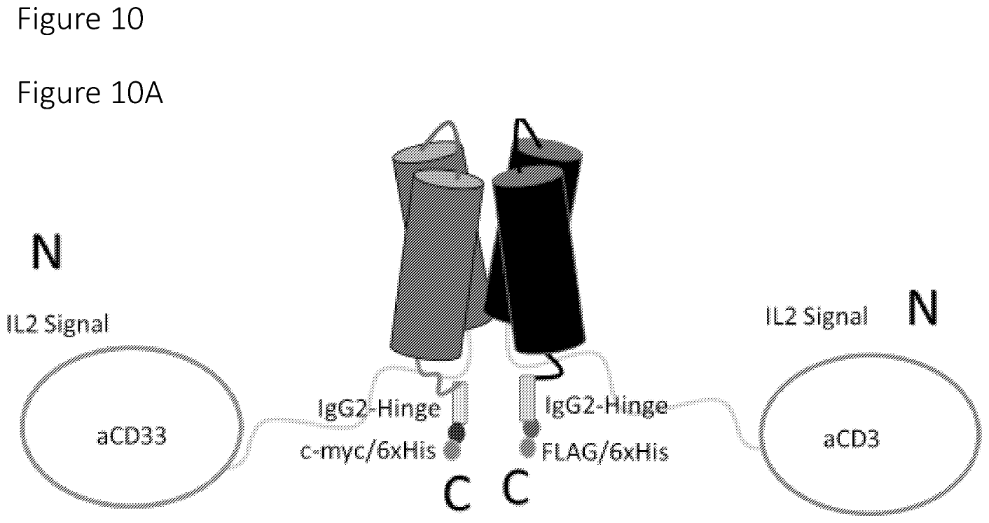

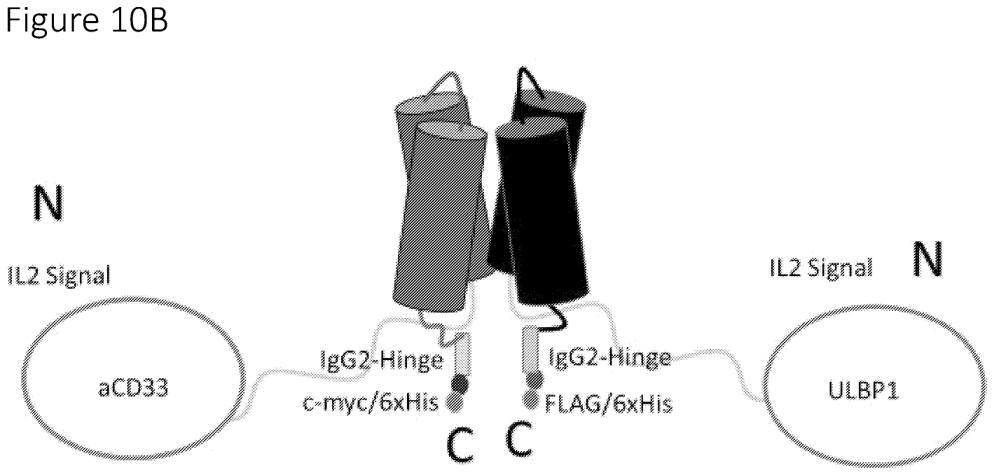

In certain embodiments, the first engineered heterodimer protein comprises: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprising a polypeptide that binds a molecule expressed on natural killer (NK) cells, a non-naturally occurring polypeptide domain comprising 1-5 alpha helices connected by amino acid linkers, and a second covalent dimerization domain as described herein. See FIGS. 1 and 10B.

In certain embodiments, antigen-binding fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment, an isolated CDR, diabodies, triabodies, tetrabodies, linear antibodies, single-chain antibody molecules. In some embodiments, scFv comprises a heavy chain variable region (V_{H}), and a light chain variable region (V_{L}).

In some embodiments, the non-naturally occurring polypeptide domain comprising 1-5 alpha helices is 6DMPa (Chen et al. 2019). In some embodiments, the non-naturally occurring polypeptide domain comprising 1-5 alpha helices is 6DMPb (Chen et al. 2019).

In some embodiments, the covalent dimerization domains are IgG2 hinge domains. In some embodiments, the covalent dimerization domains are IgG2 domains and IgG2 Fc domains. In some embodiments, the Fc domains are CH2 and CH3 domains.

The first engineered heterodimer protein can be designed to place the functional moieties (an antigen-binding fragment that binds a lineage-specific cell-surface antigen, and a polypeptide that binds a molecule expressed on NK cells) in any order. In certain embodiments, the antigen-binding fragment that binds a lineage-specific cell-surface antigen is located at the N-terminus or C-terminus of the fusion polypeptide. In certain embodiments, the polypeptide that binds a molecule expressed on natural killer (NK) cells is located at the C-terminus or N-terminus of the fusion polypeptide.

In some embodiments, the first engineered heterodimer protein comprises, from N-terminus to C-terminus, a scFv that binds to the lineage-specific cell-surface antigen (e.g., CD33 or CD19), and an ectodomain of ULBP1 (or an ectodomain of ULBP2, ULBP3, ULBP4, ULBP5, ULBP6, MICA, or MICB). In some embodiments, the fusion polypeptide comprises, from N terminus to C terminus, an ectodomain of ULBP1 (or an ectodomain of ULBP2, ULBP3, ULBP4, ULBP5, ULBP6, MICA, or MICB), and a scFv that binds to the lineage-specific cell-surface antigen (e.g., CD33 or CD19).

The first engineered heterodimer protein may further comprise a signal sequence, and/or one or more linkers. In the fusion polypeptide, these functional moieties may be covalently ligated continuously or non-continuously (e.g., they may be separated by linkers). The linker may have up to 50, up to 40, up to 30, up to 20, up to 18, up to 15, up to 12, up to 11, or up to 10, amino acid residues in length. In certain embodiments, the linker has about 1, 2, 3, 4, 5, 6, 7, 8, 9, 10-20, 8-10, 8-12, 8-15, 8-20, or 8-30 amino acid residues in length. In certain embodiments, the linker has about 7-10, 7-12, 7-15, 7-20, or 7-30 amino acid residues in length.

One type of derivatized protein is produced by crosslinking two or more polypeptides (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and phycoerythrin. A polypeptide can also be derivatized with detectable enzymes, such as alkaline phosphatase, horseradish peroxidase, beta-galactosidase, acetylcholinesterase, glucose oxidase and the like. A polypeptide can also be derivatized with a prosthetic group (e.g., streptavidin/biotin and avidin/biotin).

The first engineered heterodimer protein can be derivatized or linked to another functional molecule. For example, first engineered heterodimer protein can be functionally linked (by chemical coupling, genetic fusion, noncovalent interaction) to one or more other molecular entities, such as an antibody or antibody fragment, a detectable agent, an immunosuppressant, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof.

The first engineered heterodimer protein may further comprise a fragment (e.g., a tag) useful for polypeptide production and/or detection, including, but not limited to, poly-histidine (e.g., six histidine residues), a maltose binding protein, GST, green fluorescent protein (GFP), hemagglutinin, or alkaline phosphatase, secretion signal peptides (e.g., preprotyrypsin signal sequence), Myc, and/or FLAG.

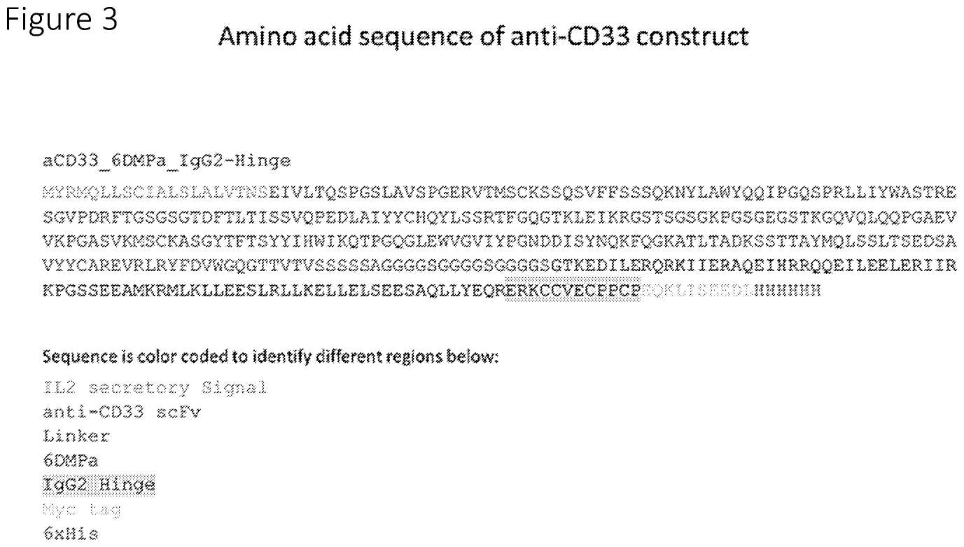

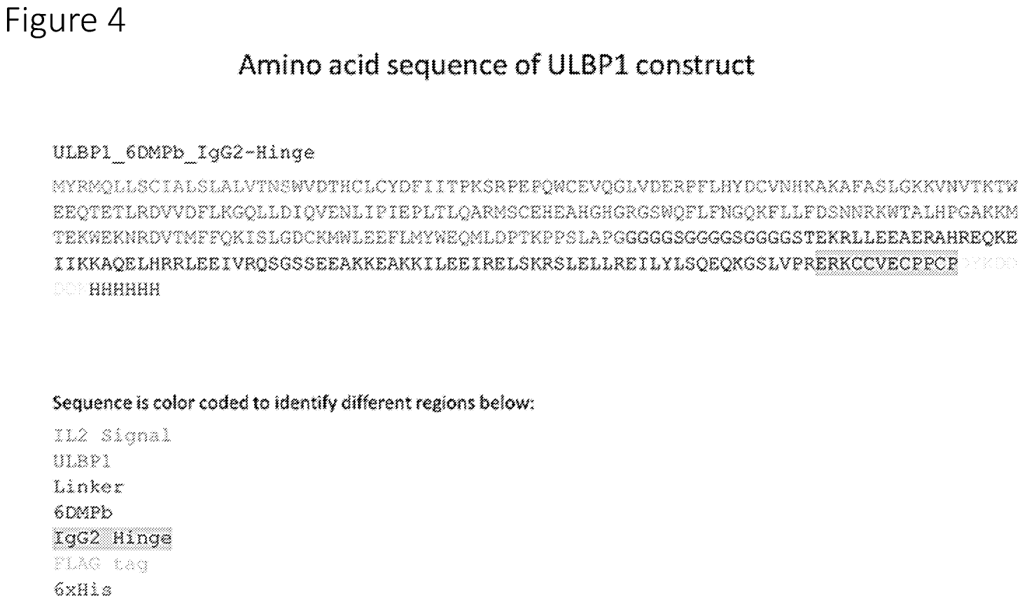

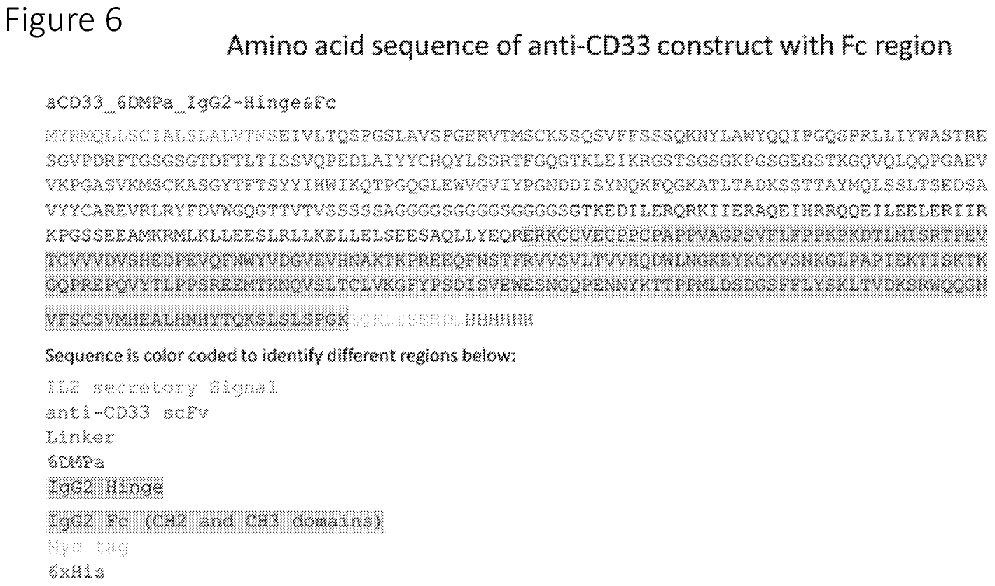

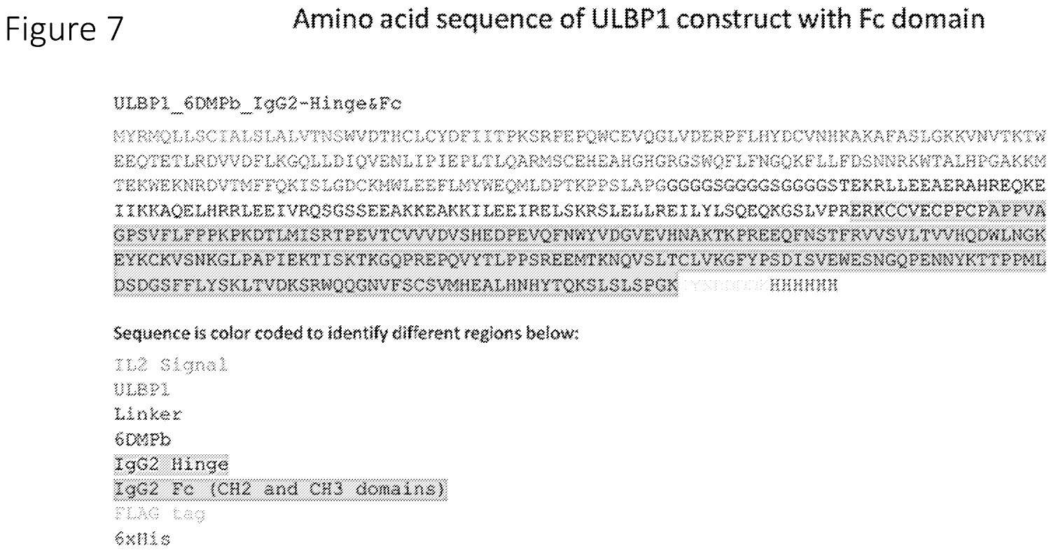

In one embodiment, the first engineered heterodimer protein comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 1 (FIG. 3) and SEQ ID NO: 2 (FIG. 4) or SEQ ID NO: 4 (FIG. 6) and SEQ ID NO: 5 (FIG. 7).

In one embodiment, the first engineered heterodimer protein comprises a signal sequence comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to an IL2 secretory signal sequence: MYRMQLLSCIALSLALVTNS (SEQ ID NO:7) (FIGS. 3, 4, 6 and 8).

In one embodiment, the first engineered heterodimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to Myc: EQKLISEEDL (SEQ ID NO: 8) (FIGS. 3 and 6).

In one embodiment, the first engineered heterodimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to FLAG: DYKDDDDK (SEQ ID NO: 14) (FIGS. 4 and 7)

In one embodiment, the first engineered heterodimer protein comprises an anti-CD33 scFv comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the anti-CD33 scFv disclosed in U.S. Patent Publication No. 20130078241.

In one embodiment, the first engineered heterodimer protein comprises an antigen-binding fragment that binds CD33, where the antigen-binding fragment comprises a light chain variable region (V_{L}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 9 (FIGS. 3 and 6).

Anti-CD33 Light Chain variable region (V_{L}) (SEQ
ID NO: 9):
EIVLTQSPGSLAVSPGERVTMSCKSSQSVFFSSSQKNYLAWYQQIPGQS
PRLLIYWASTRESGVPDRFTGSGSGTDFTLTISSVQPEDLAIYYCHQYL
SSRTFGQGTKLEIKR

In one embodiment, the first engineered heterodimer protein comprises an antigen-binding fragment that binds CD33, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 10 (FIGS. 3 and 6):

Anti-CD33 Heavy Chain variable region (V_{H}) (SEQ
ID NO: 10):
QVQLQQPGAEVVKPGASVKMSCKASGYTFTSYYIHWIKQTPGQGLEWVG
VIYPGNDDISYNQKFQGKATLTADKSSTTAYMQLSSLTSEDSAVYYCAR
EVRLRYFDVWGQGTTVTVSSSSSA

In certain embodiments, the polypeptide that binds a molecule expressed on natural killer (NK) cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence of the full-length, or a fragment, of wildtype ULBP1, ULBP2, ULBP3, ULBP4, ULBP5, ULBP6, MICA, MICB, or HCMV UL18 (including human ULBP1, ULBP2, ULBP3, ULBP4, ULBP5, ULBP6, MICA, MICB, or HCMV UL18), or of the full-length, or a fragment, of the human homolog of Raet1a, Raet1b, Raet1c, Raet1d, Raet1e, H60b, H60c.

In one embodiment, human ULBP1 has a UniProt accession number Q9BZM6. In one embodiment, an ectodomain human ULBP1 comprises (or consists essentially of, or consists of) amino acid residues 27 to 216 of Q9BZM6-1.

In one embodiment, the first engineered heterodimer protein comprises a ULBP1 ectodomain comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 15 (FIGS. 4 and 7):

ULBP1 ectodomain (27 to 216 aa of Q9BZM6-1) (SEQ
ID NO: 15):
WVDTHCLCYDFIITPKSRPEPQWCEVQGLVDERPFLHYDCVNHKAKAFA
SLGKKVNVTKTWEEQTETLRDVVDFLKGQLLDIQVENLIPIEPLTLQAR
MSCEHEAHGHGRGSWQFLFNGQKFLLFDSNNRKWTALHPGAKKMTEKWE
KNRDVTMFFQKISLGDCKMWLEEFLMYWEQMLDPTKPPSLAPG

In one embodiment, the first engineered heterodimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 12 (FIGS. 3 and 6).

6DMPa (SEQ ID NO: 12):
GTKEDILERQRKIIERAQEIHRRQQEILEELERIIRKPGSSEEAMKRML
KLLEESLRLLKELLELSEESAQLLYEQR

In one embodiment, the first engineered heterodimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 16 (FIGS. 4 and 7).

6DMPb (SEQ ID NO: 16):
TEKRLLEEAERAHREQKEIIKKAQELHRRLEEIVRQSGSSEEAKKEAKK
ILEEIRELSKRSLELLREILYLSQEQKGSLVPR

In one embodiment, the first engineered heterodimer protein comprises one or more covalent dimerization IgG2 hinge domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to IgG2 hinge: ERKCCVECPPCP (SEQ ID NO: 13) (FIGS. 3, 4, 6, and 7).

In one embodiment, the first engineered heterodimer protein comprises one or more covalent dimerization IgG2 Fc domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 19 (FIGS. 6 and 7).

IgG2 Fc Domain (SEQ ID NO: 19):
APPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIS
VEWESNGQPENNYKTTPPMLDSDGSFFLYSKLTVDKSRWQQGNVFSCSV
MHEALHNHYTQKSLSLSPGK

In one embodiment, the first engineered heterodimer protein comprises one or more linkers comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 11: GGGGSGGGGSGGGGS (FIGS. 3, 4, 6, and 7).

In further embodiments, the first engineered heterodimer protein comprises a His6 tag (HHHHHH; SEQ ID NO: 20).

In certain embodiments, the second engineered heterodimer protein comprises: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprising a polypeptide that binds a molecule expressed on T cells, a non-naturally occurring polypeptide domain comprising 1-5 alpha helices connected by amino acid linkers, and a second covalent dimerization domain as described herein.

In certain embodiments, antigen-binding fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment, an isolated CDR, diabodies, triabodies, tetrabodies, linear antibodies, single-chain antibody molecules. In some embodiments, scFv comprises a heavy chain variable region (V_{H}), and a light chain variable region (V_{L}).

In some embodiments, the non-naturally occurring polypeptide domain comprising 1-5 alpha helices is 6DMPa (Chen et al. 2019). In some embodiments, the non-naturally occurring polypeptide domain comprising 1-5 alpha helices is 6DMPb (Chen et al. 2019).

In some embodiments, the covalent dimerization domains are IgG2 hinge domains. In some embodiments, the covalent dimerization domains are IgG2 domains and IgG2 Fc domains. In some embodiments, the Fc domains are CH2 and CH3 domains.

The second engineered heterodimer protein can be designed to place the functional moieties (an antigen-binding fragment that binds a lineage-specific cell-surface antigen, and a polypeptide that binds a molecule expressed on T cells) in any order. In certain embodiments, the antigen-binding fragment that binds a lineage-specific cell-surface antigen is located at the N-terminus or C-terminus of the fusion polypeptide. In certain embodiments, the polypeptide that binds a molecule expressed on T cells is located at the C-terminus or N-terminus of the fusion polypeptide.

In some embodiments, the second engineered heterodimer protein comprises, from N-terminus to C-terminus, a scFv that binds to the lineage-specific cell-surface antigen (e.g., CD33 or CD19), and a polypeptide that binds a molecule expressed on T cells (e.g., an anti-CD3 monoclonal antibody). In some embodiments, the fusion polypeptide comprises, from N terminus to C terminus, a polypeptide that binds a molecule expressed on T cells (e.g., an anti-CD3 mono clonal antibody). and a scFv that binds to the lineage-specific cell-surface antigen (e.g., CD33 or CD19).

The second engineered heterodimer protein may further comprise a signal sequence, and/or one or more linkers. In the second engineered heterodimer, these functional moieties may be covalently ligated continuously or non-continuously (e.g., they may be separated by linkers). The linker may have up to 50, up to 40, up to 30, up to 20, up to 18, up to 15, up to 12, up to 11, or up to 10, amino acid residues in length. In certain embodiments, the linker has about 1, 2, 3, 4, 5, 6, 7, 8, 9, 10-20, 8-10, 8-12, 8-15, 8-20, or 8-30 amino acid residues in length. In certain embodiments, the linker has about 7-10, 7-12, 7-15, 7-20, or 7-30 amino acid residues in length.

One type of derivatized protein is produced by crosslinking two or more polypeptides (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A polypeptide can also be derivatized with detectable enzymes, such as alkaline phosphatase, horseradish peroxidase, beta-galactosidase, acetylcholinesterase, glucose oxidase and the like. A polypeptide can also be derivatized with a prosthetic group (e.g., streptavidin/biotin and avidin/biotin).

The second engineered heterodimer protein can be derivatized or linked to another functional molecule. For example, second engineered heterodimer protein can be functionally linked (by chemical coupling, genetic fusion, noncovalent interaction, etc.) to one or more other molecular entities, such as an antibody or antibody fragment, a detectable agent, an immunosuppressant, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof.

The second engineered heterodimer protein may further comprise a fragment (e.g., a tag) useful for polypeptide production and/or detection, including, but not limited to, poly-histidine (e.g., six histidine residues), a maltose binding protein, GST, green fluorescent protein (GFP), hemagglutinin, or alkaline phosphatase, secretion signal peptides (e.g., preprotyrypsin signal sequence), Myc, and/or FLAG.

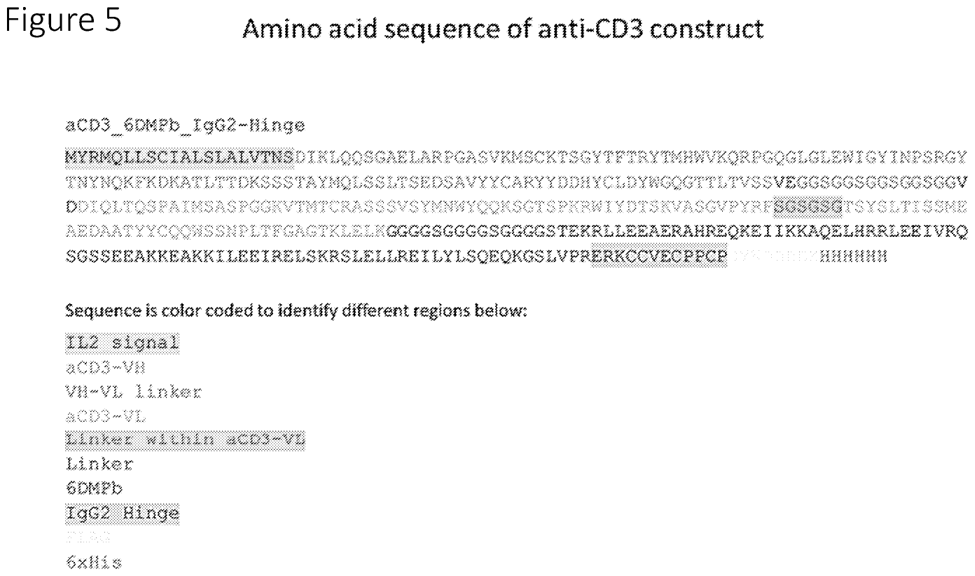

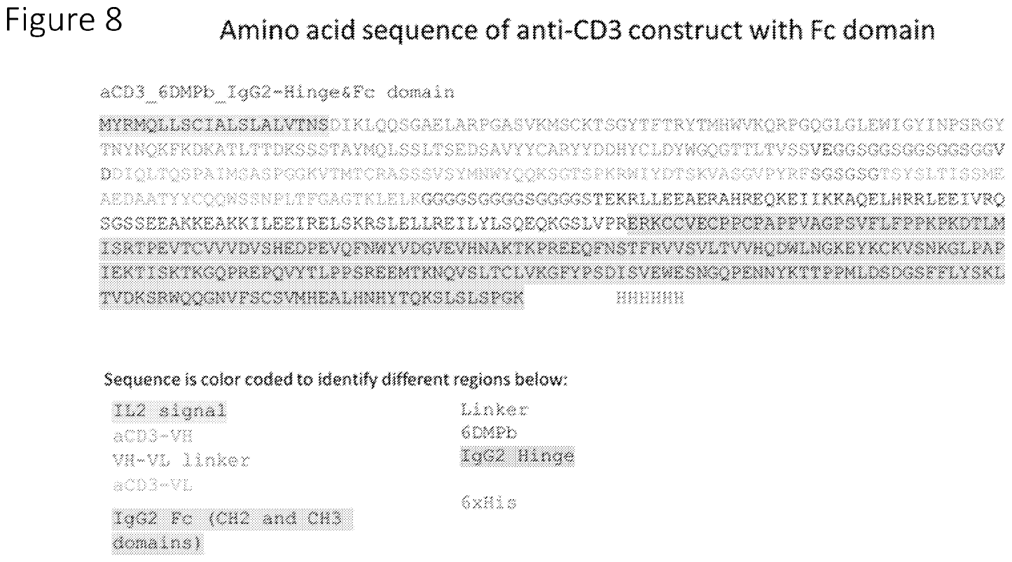

In one embodiment, the second engineered heterodimer protein comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 1 (FIG. 3) and SEQ ID NO: 3 (FIG. 5) or SEQ ID NO: 4 (FIG. 6) and SEQ ID NO: 6 (FIG. 8).

In one embodiment, the second engineered heterodimer protein comprises a signal sequence comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to an IL2 secretory signal sequence: MYRMQLLSCIALSLALVTNS (SEQ ID NO:7) (FIGS. 3, 5, 6 and 8).

In one embodiment, the second engineered heterodimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to Myc: EQKLISEEDL (SEQ ID NO: 8) (FIGS. 3 and 6).

In one embodiment, the second engineered heterodimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to FLAG: DYKDDDDK (SEQ ID NO: 14) (FIGS. 5 and 8)

In one embodiment, the second engineered heterodimer protein comprises an anti-CD33 scFv comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the anti-CD33 scFv disclosed in U.S. Patent Publication No. 20130078241.

In one embodiment, the second engineered heterodimer protein comprises an antigen-binding fragment that binds CD33, where the antigen-binding fragment comprises a light chain variable region (V_{L}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 9 (FIGS. 3 and 6).

Anti-CD33 Light Chain variable region (V_{L}) (SEQ
ID NO: 9):
EIVLTQSPGSLAVSPGERVTMSCKSSQSVFFSSSQKNYLAWYQQIPGQS
PRLLIYWASTRESGVPDRFTGSGSGTDFTLTISSVQPEDLAIYYCHQYL
SSRTFGQGTKLEIKR

In one embodiment, the second engineered heterodimer protein comprises an antigen-binding fragment that binds CD33, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 10 (FIGS. 3 and 6):

Anti-CD33 Heavy Chain variable region (V_{H}) (SEQ
ID NO: 10):
QVQLQQPGAEVVKPGASVKMSCKASGYTFTSYYIHWIKQTPGQGLEWVG
VIYPGNDDISYNQKFQGKATLTADKSSTTAYMQLSSLTSEDSAVYYCAR
EVRLRYFDVWGQGTTVTVSSSSSA

In certain embodiments, the polypeptide that binds a molecule expressed on T cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence of SEQ ID NOs: 17 and 18.

In one embodiment, the second engineered heterodimer protein comprises an antigen-binding fragment that binds CD3, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 17 (FIGS. 5 and 8):

Anti-CD3 Heavy Chain variable region (V_{H}) (SEQ
ID NO: 17):
DIKLQQSGAELARPGASVKMSCKTSGYTFTRYTMHWVKQRPGQGLGLEW
IGYINPSRGYTNYNQKFKDKATLTTDKSSSTAYMQLSSLTSEDSAVYYC
ARYYDDHYCLDYWGQGTTLTVSS

In one embodiment, the second engineered heterodimer protein comprises an antigen-binding fragment that binds CD3, where the antigen-binding fragment comprises a light chain variable region (VL) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 18 (FIGS. 5 and 8):

Anti-CD3 Light Chain variable region (V_{H}) (SEQ
ID NO: 18):
DIQLTQSPAIMSASPGGKVTMTCRASSSVSYMNWYQQKSGTSPKRWIYD
TSKVASGVPYRFTSYSLTISSMEAEDAATYYCQQWSSNPLTFGAGTKLE
LK

In one embodiment, the second engineered heterodimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 12 (FIGS. 3 and 6).

6DMPa (SEQ ID NO: 12):
GTKEDILERQRKIIERAQEIHRRQQEILEELERIIRKPGSSEEAMKRML
KLLEESLRLLKELLELSEESAQLLYEQR

In one embodiment, the second engineered heterodimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 16 (FIGS. 5 and 8).

6DMPb (SEQ ID NO: 16):
TEKRLLEEAERAHREQKEIIKKAQELHRRLEEIVRQSGSSEEAKKEAKK
ILEEIRELSKRSLELLREILYLSQEQKGSLVPR

In one embodiment, the second engineered heterodimer protein comprises one or more covalent dimerization IgG2 hinge domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to IgG2 hinge: ERKCCVECPPCP (SEQ ID NO: 13) (FIGS. 3, 5, 6 and 8).

In one embodiment, the second engineered heterodimer protein comprises one or more covalent dimerization IgG2 Fc domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 19. (FIGS. 6 and 8).

IgG2 Fc Domain (SEQ ID NO: 19):
APPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIS
VEWESNGQPENNYKTTPPMLDSDGSFFLYSKLTVDKSRWQQGNVFSCSV
MHEALHNHYTQKSLSLSPGK

In one embodiment, the second engineered heterodimer protein comprises one or more linkers comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 11: GGGGSGGGGSGGGGS (FIGS. 3-8) or to SEQ ID NO: 21: GGSGGSGGSGGSGG (FIGS. 5 and 8, CD3 VH-VL linker) or to SEQ ID NO: 22: SGSGSG (FIGS. 5 and 8, linker within in CD3-VL) In further embodiments, the second engineered heterodimer protein comprises a His6 tag (HHHHHH; SEQ ID NO: 20).

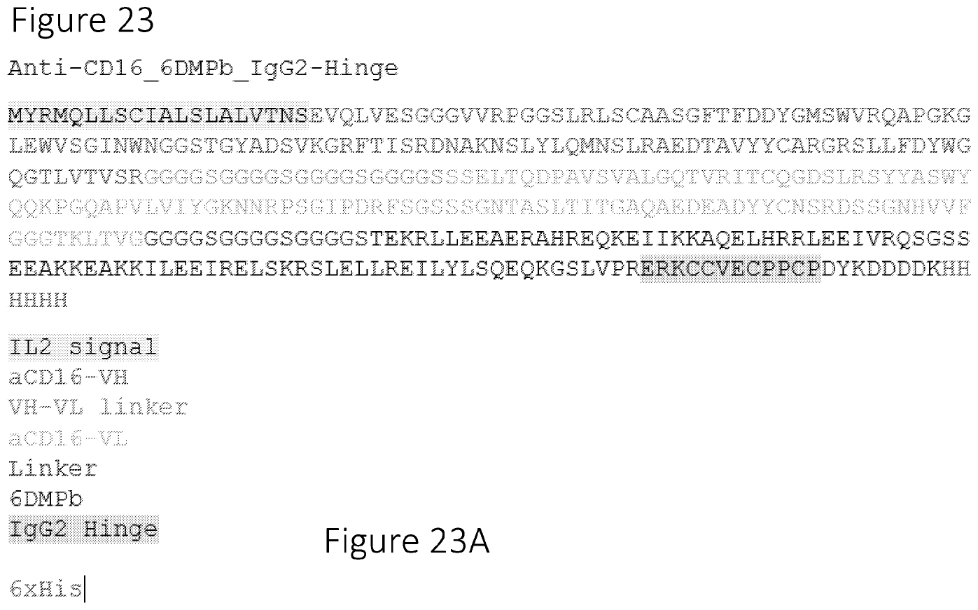

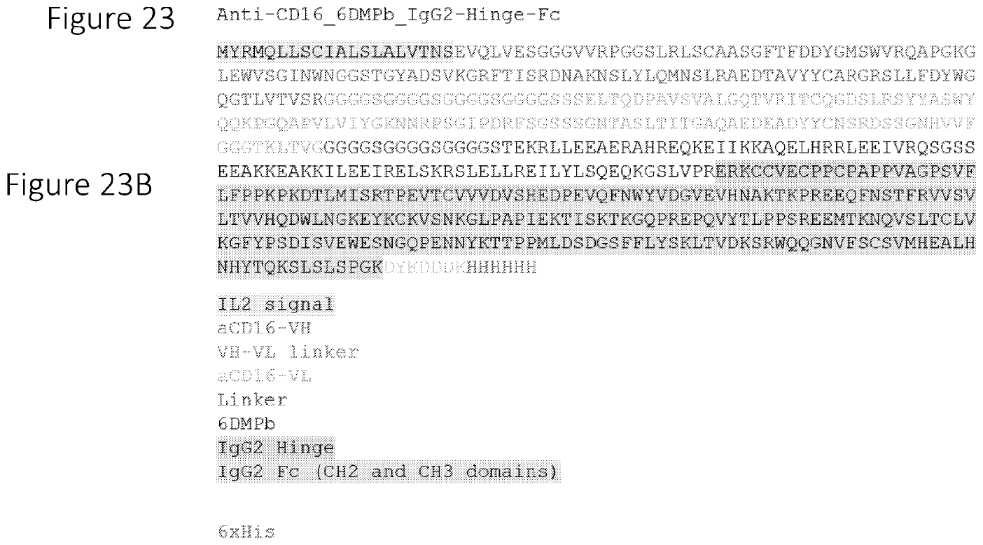

In certain embodiments, the disclosure provides for a third engineered heterodimer protein which comprises: (i) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and (ii) a second polypeptide comprising a polypeptide that binds a molecule expressed on natural killer (NK) cells, a non-naturally occurring polypeptide domain comprising 1-5 alpha helices connected by amino acid linkers, and a second covalent dimerization domain as described herein. See FIG. 23.

In certain embodiments, antigen-binding fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment, an isolated CDR, diabodies, triabodies, tetrabodies, linear antibodies, single-chain antibody molecules. In some embodiments, scFv comprises a heavy chain variable region (V_{H}), and a light chain variable region (V_{L}).

In some embodiments, the non-naturally occurring polypeptide domain comprising 1-5 alpha helices is 6DMPa (Chen et al. 2019). In some embodiments, the non-naturally occurring polypeptide domain comprising 1-5 alpha helices is 6DMPb (Chen et al. 2019).

In some embodiments, the covalent dimerization domains are IgG2 hinge domains. In some embodiments, the covalent dimerization domains are IgG2 domains and IgG2 Fc domains. In some embodiments, the Fc domains are CH2 and CH3 domains.

The third engineered heterodimer protein can be designed to place the functional moieties (an antigen-binding fragment that binds a lineage-specific cell-surface antigen, and a polypeptide that binds a molecule expressed on NK cells) in any order. In certain embodiments, the antigen-binding fragment that binds a lineage-specific cell-surface antigen is located at the N-terminus or C-terminus of the fusion polypeptide. In certain embodiments, the polypeptide that binds a molecule expressed on natural killer (NK) cells is located at the C-terminus or N-terminus of the fusion polypeptide.

In some embodiments, the third engineered heterodimer protein comprises, from N-terminus to C-terminus, a scFv that binds to the lineage-specific cell-surface antigen (e.g., CD33 or CD19), and a polypeptide that binds a molecule expressed on NK cells (e.g., an anti-CD16 monoclonal antibody). In some embodiments, the fusion polypeptide comprises, from N terminus to C terminus, a polypeptide that binds a molecule expressed on NK cells (e.g., an anti-CD16 monoclonal antibody) and a scFv that binds to the lineage-specific cell-surface antigen (e.g., CD33 or CD19).

The third engineered heterodimer protein may further comprise a signal sequence, and/or one or more linkers. In the fusion polypeptide, these functional moieties may be covalently ligated continuously or non-continuously (e.g., they may be separated by linkers). The linker may have up to 50, up to 40, up to 30, up to 20, up to 18, up to 15, up to 12, up to 11, or up to 10, amino acid residues in length. In certain embodiments, the linker has about 1, 2, 3, 4, 5, 6, 7, 8, 9, 10-20, 8-10, 8-12, 8-15, 8-20, or 8-30 amino acid residues in length. In certain embodiments, the linker has about 7-10, 7-12, 7-15, 7-20, or 7-30 amino acid residues in length.

One type of derivatized protein is produced by crosslinking two or more polypeptides (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and phycoerythrin. A polypeptide can also be derivatized with detectable enzymes, such as alkaline phosphatase, horseradish peroxidase, beta-galactosidase, acetylcholinesterase, glucose oxidase and the like. A polypeptide can also be derivatized with a prosthetic group (e.g., streptavidin/biotin and avidin/biotin).

The third engineered heterodimer protein can be derivatized or linked to another functional molecule. For example, the third engineered heterodimer protein can be functionally linked (by chemical coupling, genetic fusion, noncovalent interaction) to one or more other molecular entities, such as an antibody or antibody fragment, a detectable agent, an immunosuppressant, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof.

The third engineered heterodimer protein may further comprise a fragment (e.g., a tag) useful for polypeptide production and/or detection, including, but not limited to, poly-histidine (e.g., six histidine residues), a maltose binding protein, GST, green fluorescent protein (GFP), hemagglutinin, or alkaline phosphatase, secretion signal peptides (e.g., preprotyrypsin signal sequence), Myc, and/or FLAG.

In one embodiment, the third engineered heterodimer protein comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 1 (FIG. 3) and SEQ ID NO: 26 (FIG. 23A) or SEQ ID NO: 4 (FIG. 6) and SEQ ID NO: 27 (FIG. 23B).

In one embodiment, the third engineered heterodimer protein comprises a signal sequence comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to an IL2 secretory signal sequence: MYRMQLLSCIALSLALVTNS (SEQ ID NO:7) (FIGS. 3, 4, and 23).

In one embodiment, the third engineered heterodimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to Myc: EQKLISEEDL (SEQ ID NO: 8) (FIGS. 3 and 6).

In one embodiment, the third engineered heterodimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to FLAG: DYKDDDDK (SEQ ID NO: 14) (FIG. 23)

In one embodiment, the third engineered heterodimer protein comprises an anti-CD33 scFv comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the anti-CD33 scFv disclosed in U.S. Patent Publication No. 20130078241.

In one embodiment, the third engineered heterodimer protein comprises an antigen-binding fragment that binds CD33, where the antigen-binding fragment comprises a light chain variable region (V_{L}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 9 (FIGS. 3 and 6).

Anti-CD33 Light Chain variable region (V_{L}) (SEQ
ID NO: 9):
EIVLTQSPGSLAVSPGERVTMSCKSSQSVFFSSSQKNYLAWYQQIPGQS
PRLLIYWASTRESGVPDRFTGSGSGTDFTLTISSVQPEDLAIYYCHQYL
SSRTFGQGTKLEIKR

In one embodiment, the third engineered heterodimer protein comprises an antigen-binding fragment that binds CD33, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 10 (FIGS. 3 and 6):

Anti-CD33 Heavy Chain variable region (V_{H}) (SEQ
ID NO: 10):
QVQLQQPGAEVVKPGASVKMSCKASGYTFTSYYIHWIKQTPGQGLEWVG
VIYPGNDDISYNQKFQGKATLTADKSSTTAYMQLSSLTSEDSAVYYCAR
EVRLRYFDVWGQGTTVTVSSSSSA

In certain embodiments, the polypeptide that binds a molecule expressed on NK cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence of SEQ ID NOs: 28 and 29.

In one embodiment, the second engineered heterodimer protein comprises an antigen-binding fragment that binds CD16, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 28 (FIG. 23):

Anti-CD16 Heavy Chain variable region (V_{H}) (SEQ
ID NO: 28):
EVOLVESGGGVVRPGGSLRLSCAASGFTFDDYGMSWVRQAPGKGLEWVS
GINWNGGSTGYADSVKGRFTISRDNAKNSLYLQMNSLRAEDTAVYYCAR
GRSLLFDYWGQGTLVTVSR

In one embodiment, the third engineered heterodimer protein comprises an antigen-binding fragment that binds CD16, where the antigen-binding fragment comprises a light chain variable region (VL) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 29 (FIG. 23).

Anti-CD16 Light Chain variable region (V_{H}) (SEQ
ID NO: 29):
SSELTQDPAVSVALGQTVRITCQGDSLRSYYASWYQQKPGQAPVLVIYG
KNNRPSGIPDRFSGSSSGNTASLTITGAQAEDEADYYCNSRDSSGNHVV
FGGGTKLTVG

In one embodiment, the third engineered heterodimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 12 (FIGS. 3 and 6).

6DMPa (SEQ ID NO: 12):
GTKEDILERQRKIIERAQEIHRRQQEILEELERIIRKPGSSEEAMKRML
KLLEESLRLLKELLELSEESAQLLYEQR

In one embodiment, the third engineered heterodimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 16 (FIG. 23).

6DMPb (SEQ ID NO: 16):
TEKRLLEEAERAHREQKEIIKKAQELHRRLEEIVRQSGSSEEAKKEAKK
ILEEIRELSKRSLELLREILYLSQEQKGSLVPR

In one embodiment, the third engineered heterodimer protein comprises one or more covalent dimerization IgG2 hinge domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to IgG2 hinge: ERKCCVECPPCP (SEQ ID NO: 13) (FIGS. 3 and 23).

In one embodiment, the third engineered heterodimer protein comprises one or more covalent dimerization IgG2 Fc domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 19 (FIGS. 6 and 23).

IgG2 Fc Domain (SEQ ID NO: 19):
APPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIS
VEWESNGQPENNYKTTPPMLDSDGSFFLYSKLTVDKSRWQQGNVFSCSV
MHEALHNHYTQKSLSLSPGK

In one embodiment, the third engineered heterodimer protein comprises one or more linkers comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 11: GGGGSGGGGSGGGGS (FIGS. 3, 6, and 23) or to SEQ ID NO: 30: GGGGSGGGGSGGGGSGGGGS (FIG. 23, CD16 VH-VL linker).

In further embodiments, the third engineered heterodimer protein comprises a His6 tag (HHHHHH; SEQ ID NO: 20).

In certain embodiments, the engineered heterotrimer protein comprises: (i) a first polypeptide comprising a polypeptide that binds a molecule expressed on T cells, a non-naturally occurring polypeptide domain comprising 1-5 alpha helices connected by amino acid linkers (a1), and a first covalent dimerization domain; and (ii) a second polypeptide comprising an antigen-binding fragment that binds a lineage-specific cell-surface antigen, a non-naturally occurring polypeptide domain comprising 1-5 alpha helices connected by amino acid linkers (b1) and a second covalent dimerization domain; and (iii) a third polypeptide comprising a polypeptide that binds a molecule expressed on natural killer (NK) cells, a non-naturally occurring polypeptide domain comprising 1-5 alpha helices connected by amino acid linkers (c1), and a third second covalent dimerization domain and (iv) a fourth polypeptide comprising three non-naturally occurring polypeptide domains comprising 1-5 alpha helices connected by amino acid linkers, wherein each domain is the binding domain of a1, b1 and c1 (a2, b2 and c2), and a fourth, fifth and sixth covalent dimerization domain as described herein.

In certain embodiments, antigen-binding fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment, an isolated CDR, diabodies, triabodies, tetrabodies, linear antibodies, single-chain antibody molecules. In some embodiments, scFv comprises a heavy chain variable region (V_{H}), and a light chain variable region (V_{L}).

In some embodiments, the non-naturally occurring polypeptide domain comprising 1-5 alpha helices is 6DMPa (Chen et al. 2019). In some embodiments, the non-naturally occurring polypeptide domain comprising 1-5 alpha helices is 6DMPb (Chen et al. 2019).

In some embodiments, the covalent dimerization domains are IgG2 hinge domains. In some embodiments, the covalent dimerization domains are IgG2 domains and IgG2 Fc domains. In some embodiments, the Fc domains are CH2 and CH3 domains.

The engineered heterotrimer protein can be designed to place the functional moieties (an antigen-binding fragment that binds a lineage-specific cell-surface antigen, and a polypeptide that binds a molecule expressed on NK cells, and a polypeptide that binds a molecule expressed on T cells) in any order. In certain embodiments, the polypeptide that binds a molecule expressed on T cells antigen-binding fragment is located at the N-terminus or C-terminus of the fusion polypeptide. In certain embodiments, the polypeptide that binds a molecule expressed on natural killer (NK) cells is located at the C-terminus or N-terminus of the fusion polypeptide.

In some embodiments, the engineered heterotrimer protein comprises, from N-terminus to C-terminus, a polypeptide that binds a molecule expressed on T cells, a scFv that binds to the lineage-specific cell-surface antigen (e.g., CD33 or CD19) and an ectodomain of ULBP2, ULBP3, ULBP4, ULBP5, ULBP6, MICA, or MICB) or a polypeptide that binds CD16. In some embodiments, the fusion polypeptide comprises, from N terminus to C terminus, an ectodomain of ULBP1 (or an ectodomain of ULBP2, ULBP3, ULBP4, ULBP5, ULBP6, MICA, or MICB), a scFv that binds to the lineage-specific cell-surface antigen (e.g., CD33 or CD19) and a polypeptide that binds a molecule expressed on T cells.

The engineered heterotrimer protein may further comprise a signal sequence, and/or one or more linkers. In the fusion polypeptide, these functional moieties may be covalently ligated continuously or non-continuously (e.g., they may be separated by linkers (e.g., linker amino acid residues)). The linker may have up to 50, up to 40, up to 30, up to 20, up to 18, up to 15, up to 12, up to 11, or up to 10, amino acid residues in length. In certain embodiments, the linker has about 1, 2, 3, 4, 5, 6, 7, 8, 9, 10-20, 8-10, 8-12, 8-15, 8-20, or 8-30 amino acid residues in length. In certain embodiments, the linker has about 7-10, 7-12, 7-15, 7-20, or 7-30 amino acid residues in length.

One type of derivatized protein is produced by crosslinking two or more polypeptides (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A polypeptide can also be derivatized with detectable enzymes, such as alkaline phosphatase, horseradish peroxidase, beta-galactosidase, acetylcholinesterase, glucose oxidase and the like. A polypeptide can also be derivatized with a prosthetic group (e.g., streptavidin/biotin and avidin/biotin).

The engineered heterotrimer protein can be derivatized or linked to another functional molecule. For example, heterotrimer protein can be functionally linked (by chemical coupling, genetic fusion, noncovalent interaction, etc.) to one or more other molecular entities, such as an antibody or antibody fragment, a detectable agent, an immunosuppressant, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof.

The engineered heterotrimer protein may further comprise a fragment (e.g., a tag) useful for polypeptide production and/or detection, including, but not limited to, poly-histidine (e.g., six histidine residues), a maltose binding protein, GST, green fluorescent protein (GFP), hemagglutinin, or alkaline phosphatase, secretion signal peptides (e.g., preprotyrypsin signal sequence), Myc, and/or FLAG.

In one embodiment, the engineered heterotrimer protein comprises one or more signal sequences comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to an IL2 secretory signal sequence: MYRMQLLSCIALSLALVTNS (SEQ ID NO:7) (FIGS. 3-8).

In one embodiment, the engineered heterotrimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to Myc: EQKLISEEDL (SEQ ID NO: 8) (FIGS. 3 and 6).

In one embodiment, the engineered heterotrimer protein comprises one or more tags comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to FLAG: DYKDDDDK (SEQ ID NO: 14) (FIGS. 4, 5, 7 and 8)

In one embodiment, the engineered heterotrimer protein comprises a ULBP1 ectodomain comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 15 (FIGS. 4 and 7).

In one embodiment, the engineered heterotrimer protein comprises an anti-CD33 scFv comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the anti-CD33 scFv disclosed in U.S. Patent Publication No. 20130078241.

In certain embodiments, the polypeptide that binds a molecule expressed on NK cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence of SEQ ID NOs: 28 and 29 (FIG. 23).

In one embodiment, the engineered heterotrimer protein comprises an antigen-binding fragment that binds CD33, where the antigen-binding fragment comprises a light chain variable region (V_{L}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 9 (FIGS. 3 and 6).

In one embodiment, the engineered heterotrimer protein comprises an antigen-binding fragment that binds CD33, where the antigen-binding fragment comprises a heavy chain variable region (V_{H}) comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 10 (FIGS. 3 and 6):

In certain embodiments, the polypeptide that binds a molecule expressed on T cells comprises (or consists essentially of, or consists of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence of SEQ ID NOs: 17 and 18 (FIGS. 5 and 8).

In one embodiment, the engineered heterotrimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 12 (FIGS. 3 and 6).

In one embodiment, the engineered heterotrimer protein comprises one or more non-naturally occurring polypeptide domain comprising 1-5 alpha helices comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 16 (FIGS. 5 and 8).

In one embodiment, the engineered heterotrimer protein comprises one or more covalent dimerization IgG2 hinge domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to IgG2 hinge: ERKCCVECPPCP (SEQ ID NO: 13) (FIGS. 3-8).

In one embodiment, the engineered heterotrimer protein comprises one or more covalent dimerization IgG2 Fc domains comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 19. (FIGS. 6-8).

In one embodiment, the engineered heterotrimer protein comprises one or more linkers comprising (or consisting essentially of, or consisting of) an amino acid sequence at least or about 50%, at least about 55%, at least or about 60%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to SEQ ID NO: 11 GGGGSGGGGSGGGGS (FIGS. 3, 5, 6, and 8) or to SEQ ID NO: 21 GGGGGSGGSGGSGG or to SEQ ID NO: 22 SGSGSG (FIGS. 5 and 8)

In further embodiments, the engineered heterotrimer protein comprises a His6 tag (HHHHHH; SEQ ID NO: 20).

In some embodiments, the third polypeptide is a chemokine or cytokine protein, which increases the immune response. The chemokine or cytokine protein includes but is not limited to CXCLs including CXCL14, GCSF, and interleukins, including IL2 and IL16.

Shown herein is that the disclosed engineered proteins bind to and are cytotoxic against cells expressing CD33, such as HL60 and MOLM cells. Also shown is that when CD33 expression is increased in a cell, the binding efficiency of the engineered protein increases.

Additionally, the cytotoxicity of the engineered protein increases in cells such as MOLM, where CD33 expression is increased.

In certain embodiments, the antigen-binding fragment that binds a lineage-specific cell-surface antigen (e.g., CD33) is a derivative, or a modified form, or a variant, of a fragment of the wildtype antigen-binding fragment.

In certain embodiments, the polypeptide that binds a molecule expressed on natural killer (NK) cells is a derivative, or a modified form, or a variant, of a fragment of the wildtype polypeptide.

In certain embodiments, the polypeptide that binds a molecule expressed on T cells is a derivative, or a modified form, or a variant, of a fragment of the wildtype polypeptide.

As used herein, the term variant also denotes any peptide, pseudopeptide (peptide incorporating non-biochemical elements) or protein differing from the wildtype protein or peptide, obtained by one or more genetic and/or chemical modifications. Genetic and/or chemical modification may be understood to mean any mutation, substitution, deletion, addition and/or modification of one or more residues of the protein or peptide considered. Chemical modification may refer to any modification of the peptide or protein generated by chemical reaction or by chemical grafting of biological or non-biological molecule(s) onto any number of residues of the protein.

The present polypeptides or peptides may include variants, analogs, orthologs, homologs and derivatives of amino acids or peptides. The present polypeptides or peptides may contain one or more analogs of amino acids (including, for example, non-naturally occurring amino acids, amino acids which only occur naturally in an unrelated biological system, modified amino acids etc.), peptides with substituted linkages, as well as other modifications known in the art. The present polypeptides or peptides may comprise a peptidomimetic, such as a peptoid. The present polypeptides or peptides may contain one or more amino acid residues modified by, e.g., glycosylation, acylation (e.g., acetylation, formylation, myristoylation, palmitoylation, lipoylation), alkylation (e.g., methylation), isoprenylation or prenylation (e.g., farnesylation, geranylgeranylation), sulfation, amidation, hydroxylation, succinylation, etc. The present polypeptides and agents may be glycosylated, sulfonated and/or phosphorylated and/or grafted to complex sugars or to a lipophilic compound such as, for example, a polycarbon chain or a cholesterol derivative.

### Lineage-Specific Cell-Surface Antigens

Aspects of the disclosure provide agents targeting a lineage-specific cell-surface antigen, for example on a target cancer cell. Such an agent may comprise an antigen-binding fragment that binds and targets the lineage-specific cell-surface antigen. In some instances, the antigen-binding fragment can be a single chain antibody (scFv) specifically binding to the lineage-specific antigen. As used herein, the terms “lineage-specific cell-surface antigen” and “cell-surface lineage-specific antigen” may be used interchangeably and refer to any antigen that is sufficiently present on the surface of a cell and is associated with one or more populations of cell lineage(s). For example, the antigen may be present on one or more populations of cell lineage(s) and absent (or at reduced levels) on the cell-surface of other cell populations.

In general, lineage-specific cell-surface antigens can be classified based on a number of factors such as whether the antigen and/or the populations of cells that present the antigen are required for survival and/or development of the host organism. A summary of exemplary types of lineage-specific antigens is provide in Table 1 below.

TABLE 1
Classification of Lineage Specific Antigens

| Type of Lineage | Characteristics of the |
|---|---|
| Specific Antigen | Lineage Specific Antigen |
| Type 0 | a) antigen is required for survival of an organism |
| | and |
| | b) cell type carrying type 0 antigen is required for survival of an |
| | organism and is not unique to a tumor, or tumor-associated virus |
| Type 1 | a) antigen is not required for survival of an organism |
| | and |
| | b) cell type carrying type 1 antigen is not required for survival of |
| | an organism |
| Type 2 | a) antigen is not required for survival of an organism |
| | and |
| | b) cell type carrying type 2 antigen is required for the survival of |
| | an organism |
| Type 3 | a) antigen is not required for the survival of an organism |
| | and |
| | b) cell type carrying antigen is not required for survival of an |
| | organism |
| | c) The antigen is unique to a tumor, or a tumor associated virus. |
| | An example is the LMP-2 antigen in EBV infected cells, including |
| | EBV infected tumor cells (Nasopharyngeal carcinoma and Burkitts |
| | Lymphoma) |

Lineage specific antigens of type 1 class may be expressed in a wide variety of different tissues, including, ovaries, testes, prostate, breast, endometrium, and pancreas. In some embodiments, the agent targets a cell-surface lineage-specific antigen that is a type 1 antigen.

In some embodiments, the agent targets a cell-surface lineage-specific antigen that is a type 2 antigen. For example, CD33 is a type 2 antigen expressed in both normal myeloid cells as well as in Acute Myeloid Leukemia (AML) cells (Dohner et al. 2015).

A wide variety of antigens may be targeted by the methods and compositions of the present disclosure. Monoclonal antibodies to these antigens may be purchased commercially or generated using standard techniques, including immunization of an animal with the antigen of interest followed by conventional monoclonal antibody methodologies, e.g., the standard somatic cell hybridization technique of Kohler and Milstein, Nature (1975) 256: 495, as discussed above. The antibodies or nucleic acids encoding for the antibodies may be sequenced using any standard DNA or protein sequencing techniques.

In some embodiments, the cell-surface lineage-specific antigen that is targeted using the methods and compositions described herein is a cell-surface lineage-specific antigen of leukocytes or a subpopulation of leukocytes. In some embodiments, the cell-surface lineage-specific antigen is an antigen that is associated with myeloid cells. In some embodiments, the cell-surface lineage-specific antigen is a cluster of differentiation antigens (CDs). Examples of CD antigens include, without limitation, CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, 306, CD307a, CD307b, CD307c, D307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 and CD363.

In some embodiments, the cell-surface lineage-specific antigen is CD19, CD20, CD11, CD123, CD56, CD34, CD14, CD33, CD66b, CD41, CD61, CD62, CD235a, CD146, CD326, LMP2, CD22, CD52, CD10, CD3/TCR, CD79/BCR, and CD26. In some embodiments, the cell-surface lineage-specific antigen is CD33 or CD19.

Alternatively or in addition, the cell-surface lineage-specific antigen may be a cancer antigen, for example a cell-surface lineage-specific antigen that is differentially present on cancer cells. In some embodiments, the cancer antigen is an antigen that is specific to a tissue or cell lineage. Examples of cell-surface lineage-specific antigen that are associated with a specific type of cancer include, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), CD307e and BCMA (myeloma), RCAS1 (gynecological carcinomas, biliary adenocarcinomas and ductal adenocarcinomas of the pancreas), Claudin3, TMPRSS3 and Her2 (ovarian cancer), Her2 (breast cancer) as well as prostate specific membrane antigen. In some embodiments, the cell-surface antigen CD33 and is associated with AML cells.

In certain embodiments, the antigen-binding fragment that binds a lineage-specific cell-surface antigen (e.g., CD33) has up to or about 500, up to or about 490, up to or about 480, up to or about 470, up to or about 460, up to or about 450, up to or about 440, up to or about 430, up to or about 420, up to or about 410, up to or about 400, up to or about 390, up to or about 380, up to or about 370, up to or about 360, up to or about 350, up to or about 340, up to or about 330, up to or about 320, up to or about 310, up to or about 200, up to or about 190, up to or about 180, up to or about 170, up to or about 160, up to or about 150, up to or about 140, up to or about 130, up to or about 120, up to or about 110, up to or about 100, up to or about 90, up to or about 80, up to or about 70, up to or about 60, up to or about 50, up to or about 40, up to or about 30, up to or about 20, up to or about 15, or up to or about 10, amino acid residues in length. In certain embodiments, the antigen-binding fragment that binds a lineage-specific cell-surface antigen (e.g., CD33) has about 100-200, 80-210, 80-250, 150-250, 100-30, 50-200, 150-250, 150-300, 300-400, 200-400, 400-500, or 150-190 amino acid residues in length.

### NK Cell Surface Receptor-Binding Peptides

In certain embodiments, the present fusion polypeptide or composition comprises a polypeptide that binds to a molecule expressed on natural killer (NK) cells, such as a C-type lectin-like receptor (e.g., NKG2D).

A C-type lectin-like NK cell receptor may be a receptor expressed on the surface of natural killer cells. Exemplary NK cell receptors of this type include, but are not limited to, NKG2D (GENBANK accession number BC039836), Dectin-1 (GENBANK accession number AJ312373 or AJ312372), Mast cell function-associated antigen (GENBANK accession number AF097358), HNKR-P1A (GENBANK accession number U11276), LLT1 (GENBANK accession number AF133299), CD69 (GENBANK accession number NM_001781), CD69 homolog, CD72 (GENBANK accession number NM_001782), CD94 (GENBANK accession number NM_002262 or NM_007334), KLRF1 (GENBANK accession number NM_016523), Oxidised LDL receptor (GENBANK accession number NM 002543), CLEC-1, CLEC-2 (GENBANK accession number NM 016509), NKG2C (GENBANK accession number AJ001684), NKG2A (GENBANK accession number AF461812), NKG2E (GENBANK accession number AF461157), WUGSC:H_DJ0701016.2, or Myeloid DAP12-associating lectin (MDL-1; GENBANK accession number AJ271684). In particular embodiments, the NK cell receptor is human NKG2D or human NKG2C.

As used herein, the terms “Natural Killer Group 2D”, “NKG2D” and “NKG2D receptor”, also known as KLRK1, refer to an activating cell surface molecule that is found on numerous types of immune cells, particularly NK cells, CD8+ T cells, some CD4+ T cells, and gamma delta T cells. The terms NKG2D and NKG2D receptor include variants, isoforms, and homologs of human NKG2D receptor (see, e.g., the isoforms described in Diefenbach et al., Nat Immunol. (2002) 3(12):1142-9). NKG2D is a type II transmembrane protein with an extracellular C-type (i.e., Ca²⁺-binding) lectin-like domain but lacking the Ca²⁺ binding site. It can form heterodimers with adapter proteins such as DAP10 or DAP12, and recognizes protein ligands that include, but are not limited to, MICA, MICB, ULBP1, ULBP2, ULBP3, ULBP4, ULBP5, and ULBP6.

In certain embodiments, the NKG2D-binding peptide is an agonist of NKG2D. In certain embodiments, the NKG2D-binding peptide is an antagonist of NKG2D. In certain embodiments, the NKG2D-binding peptide is neither an antagonist nor an agonist of NKG2D.

The polypeptide that binds a molecule expressed on NK cells may be a ligand for a NK cell surface receptor, such as a ligand for the NKG2D cell surface receptor. Non-limiting examples of the ligands for NKG2D (or the NKG2D ligands) include, an MHC class I chain-related (MIC) antigen such as MICA and MICB, a UL16 binding protein (ULBP) such as ULBP1, ULBP2, ULBP3, ULBP4, ULBP5, ULBP6, and the like (Bahram Adv. Immunol. (2000) 76:1-60; Cerwenka and Lanier Immunol. Rev. (2001) 181:158-169; Cosman, et al. Immunity (2001) 14:123-133; Kubin, et al. Eur. J. Immunol. (2001) 31:1428-1437). Murine NKG2D ligands include, for example, the retinoic acid early inducible-1 gene products (RAE-1) and minor histocompatibility antigen peptide H60. NK cells can be regulated by interaction of immunomodulating polypeptide ligands with receptors on the NK cell surface. For example, ligands for the NKG2D receptor that can regulate NK cell activity, include chemokines such as muCCL21, and stress-inducible polypeptide ligands such as MHC class I chain-related antigens and ULL16 binding proteins. Murine H60 minor histocompatibility antigen peptide is reported to bind to the NKG2D receptor, as well. See, e.g., Robertson et al. Cell Immunol. (2000) 199(1):8-14; Choi et al. Immunity (2002) 17(5):593-603, and Farag et al., Blood, (2002) 100(6):1935-1947. As used herein, the term “NKG2D ligand” refers to a binding partner that binds specifically to an NKG2D receptor. Exemplary ligands include MICA, MICB, ULBP1, ULBP2, ULBP3, ULBP4, ULBP5, ULBP6, and functional fragments thereof, such as soluble forms of MIC and ULBP proteins.

Table 2 lists exemplary NKG2D binding proteins and domains.

TABLE 2
NKG2D binding proteins and domains

| | | | NKG2D |
|---|---|---|---|
| | | Uniport | binding |
| Protein | Gene names | ID | domain* |
| UL16-binding | ULBP1/RAET1I | Q9BZM6 | 27-216 |
| protein 1 | | | |
| UL16-binding | ULBP2/RAET1H | Q9BZM5 | 26-217 |
| protein 2 | | | |
| UL16-binding | ULBP3/RAET1N | Q9BZM4 | 30-217 |
| protein 3 | | | |
| UL16-binding | ULBP4/RAET1E | Q8TD07 | 31-225 |
| protein 4 | | | |
| UL-16 binding | ULBP5/RAET1G | Q6H3X3 | 26-223 |
| protein 5 | | | |
| UL16-binding | ULBP6/RAET1L | Q5VY80 | 26-218 |
| protein 6 | | | |
| MHC class I | MICA | Q29983 | 24-307 |
| polypeptide-related | | | |
| sequence A | | | |
| MHC class I | MICB | Q29980 | 23-309 |
| polypeptide-related | | | |
| sequence B | | | |
| Retinoic acid early- | Raet1a | O08602 | 29-229 |
| inducible protein 1- | | | |
| alpha | | | |
| Retinoic acid early- | Raet1b | O08603 | 29-229 |
| inducible protein 1- | | | |
| beta | | | |
| Retinoic acid early- | Raet1c | O08604 | 29-227 |
| inducible protein 1- | | | |
| gamma | | | |
| Retinoic acid early- | Raet1d | Q9JI58 | 29-225 |
| inducible protein 1- | | | |
| delta | | | |
| Retinoic acid early- | Raet1e | Q9CZQ6 | 29-225 |
| inducible protein 1- | | | |
| epsilon | | | |
| Histocompatibility | H60b | B1B212 | 25-251 |
| antigen 60b | | | |
| Histocompatibility | H60c | B1B213 | 18-177 |
| antigen 60c | | | |

*The start and end amino acids numbers are based on the sequence of protein identified by the uniport ID.

The MIC and ULBP proteins act as ligands that bind to C-type lectin-like activating receptor Natural Killer Group 2D (NKG2D) on immune effector cells, including NK cells, NKT cells, alpha beta CD8+ T cells, and gamma delta CD8+ T cells.

As used herein, the term “ULBP protein” refers to members of the MHC class I-related molecules having a characteristic organization for the unprocessed protein that includes a N-terminal signal sequence, centrally located alpha-1 and alpha-2 domains, and a C-terminal cell membrane association domain, which can be a glycosylphosphatidylinositol (GPI) anchoring domain or a transmembrane domain. Some species of ULBP protein have a cytoplasmic domain. Generally, ULBP proteins have weak amino acid sequence identity to MICA/MICB proteins. ULBP family members are ligands for the effector cell receptor NKG2D, and are known to activate NK cells. As used herein. “ULBP protein” includes active variants, isoforms, and species homologs of human ULBP protein, and includes fragments having NKG2D receptor binding activity.

As used herein, the term “ULBP1”, also described as “retinoic acid early transcript 1 protein” or “RAET1”, refers to a member of the MHC class I family, including variants, isoforms, and species homologs of human ULBP1. The protein functions as a ligand for receptor NKG2D. ULBP1 protein activates multiple signaling pathways in primary NK cells. The C terminal membrane association domain in ULBP1 comprises a GPI domain. ULBP1 is weakly homologous with MICA and MICB and has about 55% to 60% amino acid sequence identity to ULBP2 and ULBP3. Exemplary sequence of human ULBP1 is available as NCBI accession no. NP_079494.1. DNA and protein sequences for human ULBP1 have been reported by Cosman et al., Immunity (2001) 14(2):123-133, DNA Accession No. AF304377 in the EMBL database of the European Bioinformatics Institute, Wellcome Trust Genome Campus, Hinxton, Cambridge CB10 1SD, UK.

As used herein, the term “ULBP2”, also described as “retinoic acid early transcript 1H protein” or “RAET1H”, refers to a member of the MHC class I family, including variants, isoforms, and species homologs of human ULBP2. The protein functions acts as a ligand for receptor NKG2D. ULBP2 activates multiple signaling pathways in primary NK cells. The C terminal membrane association domain in ULBP2 comprises a GPI domain. ULBP2 is weakly homologous with MICA and MICB and has about 55% and 60% amino acid sequence identity to ULBP1 and ULBP3. Exemplary sequence of human ULBP2 is available as NCBI accession no. NP_079493.1. DNA and protein sequences for human ULBP2 have been reported by Cosman et al., Immunity (2001) 14(2):123-133, DNA Accession No. AF304378 in the EMBL database of the European Bioinformatics Institute, Wellcome Trust Genome Campus, Hinxton, Cambridge CB10 1SD, UK.

As used herein, the term “ULBP3”, also described as “retinoic acid early transcript 1N protein” or “RAET1N”, refers to a member of the MHC class I family, including variants, isoforms, and species homologs of human ULBP3. The protein functions as a ligand for receptor NKG2D. The C terminal membrane association domain in ULBP2 comprises a GPI anchoring domain. ULBP3 activates multiple signaling pathways in primary NK cells. ULBP3 is weakly homologous with MICA and MICB. Exemplary sequence of human ULBP3 is available as NCBI accession no. NP_078794.1. DNA and protein sequences for ULBP3 have been reported by Cosman et al., Immunity (2001) 14(2):123-133, DNA Accession No. AF304379 in the EMBL database of the European Bioinformatics Institute, Wellcome Trust Genome Campus, Hinxton, Cambridge CB10 1SD, UK.

As used herein, the term “ULBP4”, also described as “retinoic acid early transcript 1E protein” or “RAET1E”, refers to a member of the MHC class I family, including variants, isoforms, and species homologs of human ULBP4. The protein functions as a ligand for receptor NKG2D. The C terminal region of ULBP4 comprises a transmembrane domain and a cytoplasmic domain, (see, e.g., U.S. patent publication US20090274699). ULBP4 is involved in activating NK cells through its binding to receptor NKG2D and induces NK-mediated lysis (see, e.g., Kong et al., Blood (2009) 114(2):310-17). ULBP4 has higher sequence identity to ULBP3 than ULBP1 and ULBP2. Exemplary amino acid sequences of human ULBP4 are available as NCBI accession nos. NP_001230254.1; NP 001230256.1; NP 001230257.1; and NP 631904.1. As used herein, the term “ULBP5”, also described as “retinoic acid early transcript 1G protein” or “RAET1G”, refers to a member of the MHC class I family, including variants, isoforms, and species homologs of human ULBP5. The C-terminal region of the protein has a transmembrane domain and a cytoplasmic domain. ULBP5 is involved in activating NK cells and NK cell-mediated cytotoxicity through its binding to receptor NKG2D. Exemplary sequence of human ULBP5 is available as NCBI accession no. NP_001001788.2.

As used herein, the term “ULBP6”, also described as “retinoic acid early transcript 1L protein” or “RAET1L”, refers to a member of the MHC class I family, including variants, isoforms, and species homologs of human ULBP6. ULBP6 contains a GPI anchoring domain, similar to ULBP1, ULBP2, and ULBP3. ULBP6 is involved in activating NK cells and NK cell mediated cytotoxicity through its binding to receptor NKG2D. Exemplary sequence of human ULBP6 is available as NCBI accession no. NP_570970.2.

As with MICA and MICB, a known function of ULBP proteins is binding to NKG2D receptor and activating NK cell activity.

MICA is MHC class I chain-related gene A protein (MICA), including variants, isoforms, and homologs of human MICA, and includes fragments of MICA having functional MICA activity. MICA protein comprises three extracellular Ig-like domains, i.e., alpha-1, alpha-2 and alpha-3, a transmembrane domain, and an intracellular domain. The protein is expressed at low levels in cells of the gastric epithelium, endothelial cells and fibroblasts and in the cytoplasm of keratinocytes and monocytes. An exemplary sequence of MICA is available as NCBI Accession Nos. NP_000238.1. Other exemplary MICA sequences can be found in U.S. patent publication 20110311561.

MICB is MHC class I chain-related gene B protein (MICB), including variants, isoforms, and homologs of human MICB, and includes fragments of MICB having functional MICB activity. MICB has about 84% sequence identity to MICA. MICB protein comprises three extracellular Ig-like domains, i.e., alpha-1, alpha-2 and alpha-3, a transmembrane domain, and an intracellular domain. An exemplary sequence of MICB is available as UniProtKB accession number Q29980.1. Other exemplary MICB sequences can be found in U.S. patent publication 20110311561.

The NKG2D ligands (ligands for the NKG2D receptor) may also include an anti-NKG2D antibody or its fragment (e.g., an antigen-binding portion or fragment thereof), including, but not limited to, all or part of antibody that specifically recognizes or binds to NKG2D. Such antibodies can be monoclonal or polyclonal antibodies. Antibodies can also be variant antibodies, such as chimeric antibodies, humanized antibodies, single chain antibodies, and hybrid antibodies comprising immunoglobulin chains capable of binding NKG2D. In particular embodiments, the antibody comprises a single chain variable fragment. In particular embodiments, the antibody is 16F16, 16F31, MS, or 21F2, as set forth in U.S. Pat. No. 7,879,985, which is hereby incorporated by reference. The antibody fragment can be any suitable fragment as discussed herein.

In certain embodiments, the hetero protein or composition comprises a polypeptide that binds to a molecule expressed on natural killer (NK) cells that is CD16.

Such a polypeptide may comprise an antigen-binding fragment that binds and targets the molecule expressed on NK cells. In some instances, the antigen-binding fragment can be a single chain antibody (scFv) specifically binding to the molecule expressed on NK cells A wide variety of antigens may be targeted by the methods and compositions of the present disclosure. Monoclonal antibodies to these antigens may be purchased commercially or generated using standard techniques, including immunization of an animal with the antigen of interest followed by conventional monoclonal antibody methodologies, e.g., the standard somatic cell hybridization technique of Kohler and Milstein, Nature (1975) 256: 495, as discussed above. The antibodies or nucleic acids encoding for the antibodies may be sequenced using any standard DNA or protein sequencing techniques.

In certain embodiments, the polypeptide that binds a molecule expressed on natural killer (NK) cells has up to or about 500, up to or about 490, up to or about 480, up to or about 470, up to or about 460, up to or about 450, up to or about 440, up to or about 430, up to or about 420, up to or about 410, up to or about 400, up to or about 390, up to or about 380, up to or about 370, up to or about 360, up to or about 350, up to or about 340, up to or about 330, up to or about 320, up to or about 310, up to or about 200, up to or about 190, up to or about 180, up to or about 170, up to or about 160, up to or about 150, up to or about 140, up to or about 130, up to or about 120, up to or about 110, up to or about 100, up to or about 90, up to or about 80, up to or about 70, up to or about 60, up to or about 50, up to or about 40, up to or about 30, up to or about 20, up to or about 15, or up to or about 10, amino acid residues in length. In certain embodiments, the polypeptide that binds a molecule expressed on natural killer (NK) cells has about 100-200, 80-210, 80-250, 150-250, 100-30, 50-200, 150-250, 150-300, or 150-190 amino acid residues in length.

### Molecules Expressed on T Cells

Aspects of the disclosure provide agents targeting a molecule expressed on T cells. Such an agent may comprise an antigen-binding fragment that binds and targets the molecule expressed on T cells. In some instances, the antigen-binding fragment can be a single chain antibody (scFv) specifically binding to the molecule expressed on T cells A wide variety of antigens may be targeted by the methods and compositions of the present disclosure. Monoclonal antibodies to these antigens may be purchased commercially or generated using standard techniques, including immunization of an animal with the antigen of interest followed by conventional monoclonal antibody methodologies, e.g., the standard somatic cell hybridization technique of Kohler and Milstein, Nature (1975) 256: 495, as discussed above. The antibodies or nucleic acids encoding for the antibodies may be sequenced using any standard DNA or protein sequencing techniques.

In certain embodiments, the antigen-binding fragment that binds a molecule expressed on T cells lineage-specific cell-surface antigen (e.g., CD3) has up to or about 500, up to or about 490, up to or about 480, up to or about 470, up to or about 460, up to or about 450, up to or about 440, up to or about 430, up to or about 420, up to or about 410, up to or about 400, up to or about 390, up to or about 380, up to or about 370, up to or about 360, up to or about 350, up to or about 340, up to or about 330, up to or about 320, up to or about 310, up to or about 200, up to or about 190, up to or about 180, up to or about 170, up to or about 160, up to or about 150, up to or about 140, up to or about 130, up to or about 120, up to or about 110, up to or about 100, up to or about 90, up to or about 80, up to or about 70, up to or about 60, up to or about 50, up to or about 40, up to or about 30, up to or about 20, up to or about 15, or up to or about 10, amino acid residues in length. In certain embodiments, the antigen-binding fragment that binds a molecule expressed on T cells (e.g., CD3) has about 100-200, 80-210, 80-250, 150-250, 100-30, 50-200, 150-250, 150-300, 300-400, 200-400, 400-500, or 150-190 amino acid residues in length.

### Non-Naturally Occurring Polypeptide Domain

Aspects of the disclosure use a 6DMP heterodimer approach which is based on four helices—in some heterodimer designs, each protein monomer has two helices, in others, 3-to-1-which create four binding networks along the helix that ultimately favor only a heterodimer forming this network. The 6DMP heterodimerization network generates three hydrogen-bond networks and one hydrophobic core. The four helices are separated into two protein sequences, with each contributing two helices. The highly-specific four-helix structure forms only heterodimers between its partner proteins. It is known to be useful in facilitating intranuclear processes but has not been tested as a facilitator of cell-to-cell interactions.

In some embodiments, the non-naturally occurring polypeptide domain comprising 1-5 alpha helices is 6DMPa (SEQ ID NO: 12). In some embodiments, the non-naturally occurring polypeptide domain comprising 1-5 alpha helices is 6DMPb (SEQ ID NO: 16).

See Chen et al. 2019, incorporated in its entirety by reference herein.

### Antigen-Binding Fragment

The antigen-binding fragment may be an antibody fragment. The antibody or antibody fragment may be any of the immunoglobulin classes (e.g., IgA, IgD, IgE, IgG, and IgM) and subclasses, so long as they are capable of binding. In certain embodiments, the antibody fragment has an antigen-binding portion. In certain embodiments, antibody fragments include, but are not limited to, Fab, F(ab′)2, Fab′, F(ab)′, Fv, a disulfide linked Fv, single chain Fv (scFv), bivalent scFv (bi-scFv), trivalent scFv (tri-scFv), Fd, dAb fragment (e.g., Ward et al., Nature, (1989) 341:544-546), an isolated CDR, diabodies, affibodies, triabodies, tetrabodies, linear antibodies, single-chain antibody molecules. Single chain antibodies produced by joining antibody fragments using recombinant methods, or a synthetic linker, are also encompassed by the present disclosure. Bird et al. Science, (1988), 242:423-426. Huston et al., Proc. Natl. Acad. Sci. USA, (1988), 85:5879-5883. Antibody fragments comprise only a portion of an intact antibody, generally including an antigen binding site of the intact antibody and thus retaining the ability to bind antigen. Examples of antibody fragments encompassed by the present invention include: the Fab fragment, having a light chain variable domain (V_{L}), light chain constant domain (C_{L}), heavy chain variable domain (V_{H}), and heavy chain constant domain (C_{H}); the Fab′ fragment, which is a Fab fragment having one or more cysteine residues at the C-terminus of the C_{H} domain; the Fd fragment having V_{H} and C_{H} domains; the Fd′ fragment having V_{H} and C_{H} domains and one or more cysteine residues at the C-terminus of the C_{H} domain; the Fv fragment having the V_{L} and V_{H} domains of a single arm of an antibody; the dAb fragment (Ward et al., Nature (1989) 341:544-546) which consists of a V_{H} domain; isolated CDR regions; F(ab′)2 fragments, a bivalent fragment including two Fab′ fragments linked by a disulphide bridge at the hinge region; single chain antibody molecules (Bird et al., Science (1988) 242:423-426; and Huston et al., PNAS (1988) 85:5879-5883; diabodies with two antigen binding sites, comprising a V_{H} domain connected to a V_{L} domain in the same polypeptide chain (see, e.g., WO 93/11161 to Whitlow et al. and Hollinger et al., PNAS (1993) 90:6444-6448; affibodies which are triple helix high affinity peptides (see, e.g., Nygren, “FEBS Journal (2008) 275:2668-2676,), and linear antibodies comprising a pair of tandem Fd segments (VH-CH1-VH-CH1) which, together with complementary light chain polypeptides, form a pair of antigen binding regions (Zapata et al., Protein Eng. (1995) 8(10):1057-1062; U.S. Pat. Nos. 5,641,870; 8,580,755).

Any antibody or an antigen-binding fragment thereof can be used for constructing the agent that targets a lineage-specific cell-surface antigen as described herein. Such an antibody or antigen-binding fragment can be prepared by a conventional method, for example, the hybridoma technology or recombinant technology.

For example, antibodies specific to a lineage-specific antigen of interest can be made by the conventional hybridoma technology. The lineage-specific antigen, which may be coupled to a carrier protein such as KLH, can be used to immunize a host animal for generating antibodies binding to that complex. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, et al., In Vitro, (1982)18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TCR-like monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to a lineage-specific antigen. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the lineage-specific antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins.

Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-MouseR® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., Annu. Rev. Immunol. (1994) 12:433-455. Alternatively, the phage display technology (McCafferty et al., Nature (1990) 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., Proc. Nat. Acad. Sci. (1984) 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. Proc. Natl. Acad. Sci. USA (1984) 81:6851; Neuberger et al. Nature (1984) 312:604; and Takeda et al. Nature (1984) 314:452.

Methods for constructing humanized antibodies are also well known in the art. See, e.g., Queen et al., Proc. Natl. Acad. Sci. USA, (1989) 86:10029-10033. In one example, variable regions V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a lineage-specific antigen can be identified from the library following routine procedures. Positive clones can be subjected to further screening to identify those that bind lineage-specific antigen.

The “percent identity” of two amino acid sequences is determined using the algorithm of Karlin and Altschul Proc. Natl. Acad. Sci. USA (1990) 87:2264-68, modified as in Karlin and Altschul Proc. Natl. Acad. Sci. USA (1993) 90:5873-77. Such an algorithm is incorporated into the NBLAST and XBLAST programs (version 2.0) of Altschul, et al. J. Mol. Biol. (1990) 215:403-10. BLAST protein searches can be performed with the XBLAST program, score=50, wordlength=3 to obtain amino acid sequences homologous to the protein molecules of the present disclosure. Where gaps exist between two sequences, Gapped BLAST can be utilized as described in Altschul et al., Nucleic Acids Res. (1997) 25(17):3389-3402. When utilizing BLAST and Gapped BLAST programs, the default parameters of the respective programs (e.g., XBLAST and NBLAST) can be used.

### Nucleic Acids and Vectors

The present disclosure provides for a nucleic acid/polynucleotide encoding any of the disclosed polypeptides, engineered proteins or agents. For example, polynucleotides encoding any of the proteins described herein are provided, e.g., for recombinant expression and purification. In some embodiments, an isolated polynucleotide comprises one or more sequences encoding the fusion proteins or agents. The nucleic acid may be deoxyribonucleic acid (DNA), ribonucleic acid (RNA) or a DNA/RNA hybrid. The nucleic acid may be linear or circular (such as a plasmid). The nucleic acid may be single-stranded, double-stranded, branched or modified by the ligation of non-nucleic acid molecules. The nucleic acids include nucleic acids produced by recombinant technology.

In certain embodiments, the nucleic acid encoding the disclosed engineered proteins is codon optimized. Methods for codon optimization are known in the art.

In certain embodiments, the nucleic acid encoding the aCD33-6DMPa-Hinge polypeptide has a nucleic acid sequence that is at least 70% identical to SEQ ID NO: 23 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence in SEQ ID NO: 23).

In certain embodiments, the nucleic acid encoding the polypeptide ULBP1-6DMPb-Hinge has a nucleic acid sequence that is at least 70% identical to SEQ ID NO: 24 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence in SEQ ID NO: 24).

In certain embodiments, the nucleic acid encoding the aCD3-6DMPb polypeptide has a nucleic acid sequence that is at least 70% identical to SEQ ID NO: 25 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence in SEQ ID NO: 25).

In certain embodiments, the nucleic acid is a plasmid DNA including a coding sequence for the disclosed polypeptide or agents, together with flanking regulatory sequences effective to cause the expression of the fusion polypeptide or agents in cells. Examples of flanking regulatory sequences are a promoter sequence sufficient to initiate transcription and a terminator sequence sufficient to terminate the gene product, by termination of transcription or translation. Suitable transcriptional or translational enhancers can be included in the vector to further assist the expression of the fusion polypeptide or agents.

In some embodiments, vectors encoding any of the engineered proteins described herein are provided, e.g., for recombinant expression and purification. In some embodiments, the vector comprises or is engineered to include an isolated polynucleotide, e.g., those described herein. Typically, the vector comprises a sequence encoding the engineered polypeptide or protein or agents operably linked to a promoter, such that the engineered protein (or agents) is (are) expressed in a host cell.

The nucleic acid may be contained within an expression vector. Thus, for example, a nucleic acid sequence may be included in any one of a variety of expression vectors for expressing one or more polypeptides, and more than one nucleic acid may be included in one expression vector. Alternatively, parts of one gene or nucleic acid may be included in separate vectors. In some embodiments, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and derivatives of viral DNA).

Vectors of the present disclosure can drive the expression of one or more sequences in mammalian cells using a mammalian expression vector. Examples of mammalian expression vectors include pCDM8 (Seed, Nature (1987) 329:840) and pMT2PC (Kaufman, et al., EMBO J. (1987) 6:187). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989.

The vectors of the present disclosure may direct expression of the nucleic acid preferentially in a particular cell type (e.g., tissue-specific regulatory elements are used to express the nucleic acid). Such regulatory elements include promoters that may be tissue-specific or cell type-specific. The term “tissue-specific” as it applies to a promoter refers to a promoter that is capable of directing selective expression of a nucleotide sequence of interest to a specific type of tissue in the relative absence of expression of the same nucleotide sequence of interest in a different type of tissue. The term “cell type-specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type-specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Conventional viral and non-viral based gene transfer methods can be used to introduce nucleic acids in mammalian cells or target tissues. Such methods can be used to administer nucleic acids encoding the present agents/polypeptides to cells in culture, or in a subject. Non-viral vector delivery systems include DNA plasmids, RNA (e.g., a transcript of a vector described herein), naked nucleic acid, and nucleic acid complexed with a delivery vehicle. Viral vector delivery systems include DNA and RNA viruses, which have either episomal or integrated genomes after delivery to the cell.

Viral vectors can be administered directly to patients (in vivo) or they can be used to manipulate cells in vitro or ex vivo, where the modified cells may be administered to patients. In one embodiment, the present disclosure utilizes viral based systems including, but not limited to retroviral, lentivirus, adenoviral, adeno-associated and herpes simplex virus vectors for gene transfer. Furthermore, the present disclosure provides vectors capable of integration in the host genome, such as retrovirus or lentivirus.

The vectors of the present disclosure may be delivered to the eukaryotic cell in a subject.

Any of the chimeric proteins described herein can be prepared by routine methods, such as recombinant technology. Methods for preparing the chimeric proteins herein involve generation of a nucleic acid that encodes a polypeptide comprising each of the fragments/domains/moieties of the chimeric proteins, including the antigen-binding fragment and the polypeptide that binds a molecule expressed on natural killer (NK) cells. In some embodiments, a nucleic acid encoding each of the components of chimeric protein are joined together using recombinant technology.

Sequences of each of the components of the engineered proteins may be obtained via routine technology, e.g., PCR amplification from any one of a variety of sources known in the art. In some embodiments, sequences of one or more of the components of the chimeric proteins are obtained from a human cell. Alternatively, the sequences of one or more components of the chimeric proteins can be synthesized. Sequences of each of the components (e.g., fragments/domains/moieties) can be joined directly or indirectly (e.g., using a nucleic acid sequence encoding a peptide linker) to form a nucleic acid sequence encoding the chimeric protein, using methods such as PCR amplification or ligation. Alternatively, the nucleic acid encoding the chimeric protein may be synthesized. In some embodiments, the nucleic acid is DNA. In other embodiments, the nucleic acid is RNA.

Mutation of one or more residues within one or more of the components of the chimeric protein (e.g., the antigen-binding fragment, etc.), prior to or after joining the sequences of each of the components. In some embodiments, one or more mutations in a component of the engineered protein may be made to modulate (increase or decrease) the affinity of the component for a target (e.g., the antigen-binding fragment for the target antigen) and/or modulate the activity of the component.

Any of the engineered proteins described herein can be introduced into a suitable cell for expression via conventional technology.

To express the engineered proteins, expression vectors for stable or transient expression of the engineered proteins may be constructed via conventional methods as described herein. For example, nucleic acids encoding the chimeric proteins may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the engineered proteins. The synthetic linkers may contain nucleic acid sequences that correspond to a particular restriction site in the vector. The selection of expression vectors/plasmids/viral vectors would depend on the type of host cells for expression of the chimeric proteins, but should be suitable for integration and replication in eukaryotic cells.

A variety of promoters can be used for expression of the engineered proteins described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric proteins include any constitutively active promoter. Alternatively, any regulatable promoter may be used, such that its expression can be modulated.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric protein. See section VI below. Suitable vectors and methods for producing vectors containing transgenes are well known and available in the art. Examples of the preparation of vectors for expression of chimeric proteins can be found, for example, in US2014/0106449.

In some embodiments, the engineered protein or the nucleic acid encoding said engineered protein is a DNA molecule. In some embodiments, the nucleic acid encoding said engineered protein is a DNA vector. In some embodiments, the nucleic acid encoding the engineered protein is an RNA molecule.

Any of the vectors comprising a nucleic acid sequence that encodes an engineered protein described herein is also within the scope of the present disclosure. Such a vector may be delivered into host cells by a suitable method. Methods of delivering vectors to cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Natl. Acad. Sci. USA (2013) 110(6):2082-2087); or viral transduction. In some embodiments, the vectors for expression of the chimeric proteins are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression are retroviruses. In some embodiments, the vectors for expression are lentiviruses. In some embodiments, the vectors for expression are adeno-associated viruses.

In examples in which the vectors encoding engineered proteins are introduced to the host cells using a viral vector, viral particles that are capable of infecting the cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the cells.

### Therapeutic Methods

Any of the disclosed engineered proteins may be administered to a subject to treat a condition such as hematopoietic malignancy. Additionally, any of the nucleic acids/polynucleotides/vectors encoding the disclosed engineered proteins may be administered to a subject to treat a condition such as hematopoietic malignancy. Additionally, any compositions or pharmaceutical compositions comprising any of the disclosed engineered proteins or nucleic acids/polynucleotides/vectors encoding the disclosed engineered proteins may be administered to a subject to treat a condition such as hematopoietic malignancy. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human patient having a hematopoietic malignancy.

In some embodiments, the present vectors, engineered proteins or agents may be mixed with a pharmaceutically acceptable carrier to form a pharmaceutical composition, which is also within the scope of the present disclosure.

The present disclosure provides for a vaccine suitable for eliciting an immune response against cancer cells. Method of inhibiting tumor growth by administering the vaccine of the invention to a mammal is also described.

The present composition may be delivered to, or administered to be in contact with, any suitable types of cells. The cell may a eukaryotic cell. The cell may a mammalian cell, such as a human cell or a non-human mammalian cell (e.g., a non-human primate cell). These include a number of cell lines that can be obtained from American Tissue Culture Collection. In certain embodiments, the cell is a tumor cell.

In certain embodiments, the cell is present in a subject (e.g., a mammal). The mammal can be a human or a non-human primate. Non-human primates include, but are not limited to, chimpanzees, cynomologous monkeys, spider monkeys, and macaques, e.g., Rhesus.

In certain embodiments, the cell may be removed and maintained in tissue culture in a primary, secondary, immortalized or transformed state. In certain embodiments, the cells are cultured cells or cells freshly obtained from a source (e.g., a tissue, an organ, a subject, etc.). The mammalian cell can be primary or secondary which means that it has been maintained in culture for a relatively short time after being obtained from an animal tissue.

To perform the methods described herein, an effective amount of the present composition may be administered to a subject in need of the treatment. As used herein the term “effective amount” may be used interchangeably with the term “therapeutically effective amount” and refers to that quantity of a vector, an engineered protein, an agent, or pharmaceutical composition that is sufficient to result in a desired activity upon administration to a subject in need thereof. Within the context of the present disclosure, the term “effective amount” refers to that quantity of a vector, an engineered protein, an agent, or pharmaceutical composition that is sufficient to delay the manifestation, arrest the progression, relieve or alleviate at least one symptom of a disorder treated by the methods of the present disclosure.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human patient having a hematopoietic malignancy.

In some embodiments, the present composition is administered to a subject in an amount effective in to reduce the number of target cells (e.g., cancer cells) by least 20%, e.g., 50%, 80%, 100%, 2-fold, 5-fold, 10-fold, 20-fold, 50-fold, 100-fold or more.

In one embodiment, the present composition is administered to a subject (e.g., human patient) as an initial dose. One or more subsequent administrations of the present composition may be provided to the patient at intervals of 15 days, 14, 13, 12, 11, 10, 9, 8, 7, 6, 5, 4, 3, or 2 days after the previous administration. More than one dose of the present composition can be administered to the subject per week, e.g., 2, 3, 4, or more administrations of the agent. The subject may receive more than one doses of the present composition per week, followed by a week of no administration of the agent, and finally followed by one or more additional doses of the present composition (e.g., more than one administration of the present composition per week). The present composition may be administered every other day for 3 administrations per week for two, three, four, five, six, seven, eight or more weeks.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis.

In some embodiments, the present engineered proteins fusion recognizes (binds) a target cell expressing the cell-surface lineage-specific antigen for targeting killing.

The efficacy of the present therapeutic methods may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the cell-surface lineage-specific antigen.

The present composition may be administered to a subject in combination with a second therapy. The present composition may be administered prior to administration of the second therapy. In some embodiments, the present composition is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the second therapy.

In some embodiments, the second therapy is administered prior to the administration of the present composition. In some embodiments, the second therapy is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the present composition.

In some embodiments, the present composition and the second therapy are administered at substantially the same time. In some embodiments, the present composition is administered, and the patient is assessed for a period of time, after which the second therapy is administered. In some embodiments, the second therapy is administered, and the patient is assessed for a period of time, after which the present composition is administered.

Also within the scope of the present disclosure are multiple administrations (e.g., doses) of the present composition. In some embodiments, the present composition is administered to the subject once. In some embodiments, the present composition is administered to the subject more than once (e.g., at least 2, 3, 4, 5, or more times). In some embodiments, the present composition is administered to the subject at a regular interval, e.g., every six months.

In some embodiments, the subject is a human subject having a hematopoietic malignancy or hematological neoplasm. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukemia, acute lymphoid leukemia, chronic myelogenous leukemia, acute lymphoblastic leukemia or chronic lymphoblastic leukemia, and chronic lymphoid leukemia.

Hematological malignancies or neoplasms include but not limited to, myeloid malignancies, lymphatic malignancies, malignant histiocytosis and mast cell leukemia.

The hematopoietic malignancy may be a myeloid malignancy wherein the myeloid malignancies include but not limited to myeloproliferative disorders (MPD), myelodysplastic syndrome (MDS), myelodysplastic/myeloproliferative disorders (MD/MPD) and acute myeloid leukemia (AML).

In certain embodiments, myeloid malignancies refer to a condition associated with a defect in the proliferation of a hematopoietic cell. In certain embodiments, myeloid malignancies refer to clonal hematological diseases affecting the myeloid blood lineages, including chronic and acute conditions. Myeloid malignancies include myeloproliferative neoplasms, myelodysplastic syndromes and acute myeloid leukemias. A myeloproliferative neoplasm may be primary myelofibrosis (PMF), or essential thrombocythemia (ET). A myelodysplastic syndrome may be refractory anemia with ringed sideroblasts and thrombocythemia (RARS-T). Myeloid malignancies include, but are not limited to, myeloproliferative disorders (MPD), myelodysplastic syndrome (MDS), myelodysplastic/myeloproliferative disorders (MD/MPD), and acute myeloid leukemia (AML).

Lymphatic malignancies include, but are not limited to, T/NK cell tumor, B cell tumor and Hodgkin's disease.

In some embodiments, the leukemia is acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al. 2015). CD33 glycoprotein is expressed on the majority of myeloid leukemia cells as well as on normal myeloid and monocytic precursors and has been considered to be an attractive target for AML therapy (Laszlo et al., Blood Rev. (2014) 28(4):143-53). While clinical trials using anti-CD33 monoclonal antibody based therapy have shown improved survival in a subset of AML patients when combined with standard chemotherapy, these effects were also accompanied by safety and efficacy concerns.

Alternatively or in addition, the methods described herein may be used to treat non-hematopoietic cancers, including without limitation: lung cancer; ear, nose and throat cancer; colon cancer; melanoma; pancreatic cancer; mammary cancer; prostate cancer; breast cancer; ovarian cancer; basal cell carcinoma; biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Carcinomas are cancers of epithelial origin. Carcinomas intended for treatment with the methods of the present disclosure include, but are not limited to, acinar carcinoma, acinous carcinoma, alveolar adenocarcinoma (also called adenocystic carcinoma, adenomyoepithelioina, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Huirthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum.

Sarcomas are mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In some embodiments, the cancer to be treated can be a refractory cancer. A “refractory cancer,” as used herein, is a cancer that is resistant to the standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, or surgery, or radiation, or a combination thereof. Those of ordinary skill in the art are aware of such standards of care. Subjects being treated according to the present disclosure for a refractory cancer therefore may have already been exposed to another treatment for their cancer. Alternatively, if the cancer is likely to be refractory (e.g., given an analysis of the cancer cells or history of the subject), then the subject may not have already been exposed to another treatment. Examples of refractory cancers include, but are not limited to, leukemia, melanomas, renal cell carcinomas, colon cancer, liver (hepatic) cancers, pancreatic cancer, Non-Hodgkin's lymphoma and lung cancer.

Any of the present vectors, engineered proteins or agents described herein may be administered in a pharmaceutically acceptable carrier or excipient as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

### Kits

Also within the scope of the present disclosure are kits for use of the present engineered proteins, agents, vectors, and/or compositions. Such kits may include one or more containers comprising present engineered proteins, agents, vectors, and/or compositions.

Some aspects of this disclosure provide kits comprising the present engineered proteins or agents. In some embodiments, the kit comprises a polynucleotide encoding the present engineered proteins. In some embodiments, the kit comprises a vector for recombinant protein expression, wherein the vector comprises a polynucleotide encoding the present engineered proteins. In some embodiments, the kit comprises a cell that comprises a genetic construct for expressing the present engineered proteins. In some embodiments, the kit comprises an excipient and instructions for using the kit. In some embodiments, the excipient is a pharmaceutically acceptable excipient.

In some embodiments, the kit can comprise instructions for use in any of the methods described herein. The included instructions can comprise a description of administration of the pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the pharmaceutical composition to a subject who is in need of the treatment.

The instructions relating to the use of the pharmaceutical composition described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

In some embodiment, the disclosure provides articles of manufacture comprising contents of the kits described above.

In some embodiments, the individual components of the formulation can be provided in one container. Alternatively, it can be desirable to provide the components of the formulation separately in two or more containers. The different components can be combined, e.g., according to instructions provided with the kit. The components can be combined according to a method described herein, e.g., to prepare and administer a pharmaceutical composition.

The present engineered proteins, agents, vectors, or compositions can be provided in any form, e.g., liquid, dried or lyophilized form.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Construction of the Engineered Heterodimer Proteins

Initially, pcDNA3.1 expression vectors were used for the aCD33-ULBP1 engineered heterodimer proteins. All constructs utilized an N-terminal IL-2 secretory signal to trigger export of the completed protein to cell supernatant, and C-terminal myc- and 6×His-tags to facilitate purification and labeling. In two constructs, ULBP1 immediately follows the IL-2 signal, followed by a linker and then the VH and VL regions of aCD33. In another construct, aCD33 precedes ULBP1, in order to determine whether one orientation is favorable. Additionally, constructs have been developed for the aCD33-scFV or ULBP1 alone.

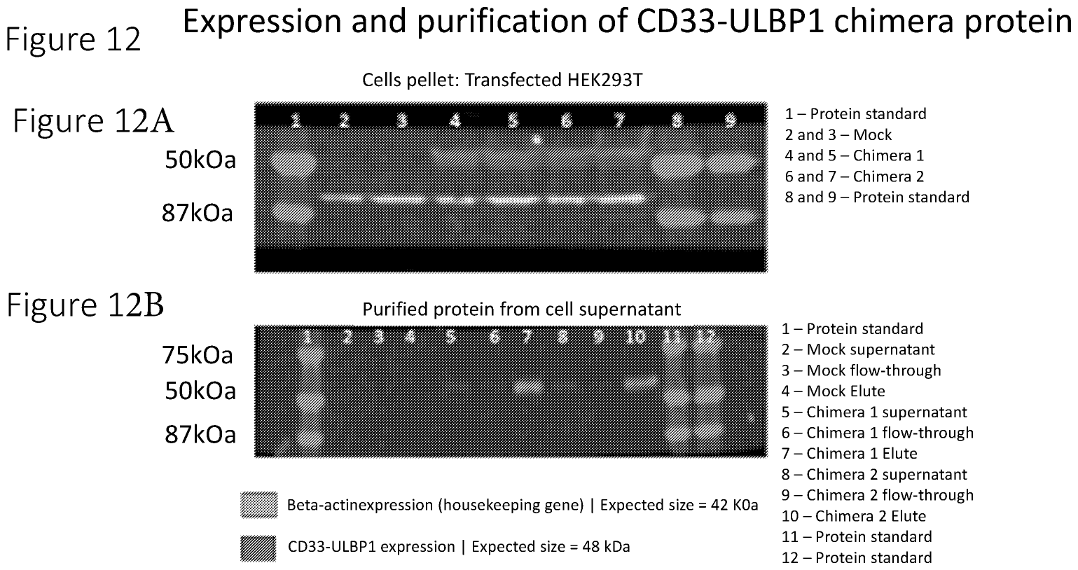

Expression via Mirus TransIT-293 transfection reagent was confirmed using Western blot probing for Myc following purification of 6×His-tagged proteins in supernatant of transfected HEK-293T cells, as well as Myc probing of cellular protein. See Example 2 and FIG. 12.

The 6DMPa-monomer was fused to anti-CD33, GFP, and the associated tags (6×His and myc) listed above. The 6DMPb monomer was fused to UPBP1, RFP, and 6×His and FLAG tags to facilitate purification independently of the 6DMPa-myc (and to allow for separate co-immunoprecipitation experiments).

pcDNA3.1 plasmids were also created that encode for the engineered protein that attaches anti-CD3 to 6DMPb, with tags to match those of the ULBP1-6DMPb construct (FIG. 10). In this approach, CD3+ T-cells replaced NK cells as the relevant effector cell, and thus 6DMPb was attached in order to bind the anti-CD3 facilitator to the anti-CD33 targeting moiety. The anti-CD3 sequence was taken from the Amgen CD3-CD19 BiTE blinotumomab, and fused to 6DMPb via the same linkers used previously (Kufer et al. 2017). This was co-expressed with aCD33-6DMPa and co-immunoprecipitated to verify heterodimerization. The construct was cloned into bacterial or lentiviral vectors to generate high-output protein expression methods as noted above.

The latest modification to these designs included the hinge region of IgG2. With four cysteine residues creating four disulfide bridges in the span of 12 total residues, hinge regions added to the C-terminus of 6DMP-based chimeras were expected to enhance the binding of heterodimers and prevent dissociation (Wypych et al. 2008). An identical IgG2-hinge was thus added to each construct, following the 6DMPa/b and preceding purification tags (FIGS. 1 and 10). Constructs have been designed that add this hinge directly or add this hinge after a short linker.

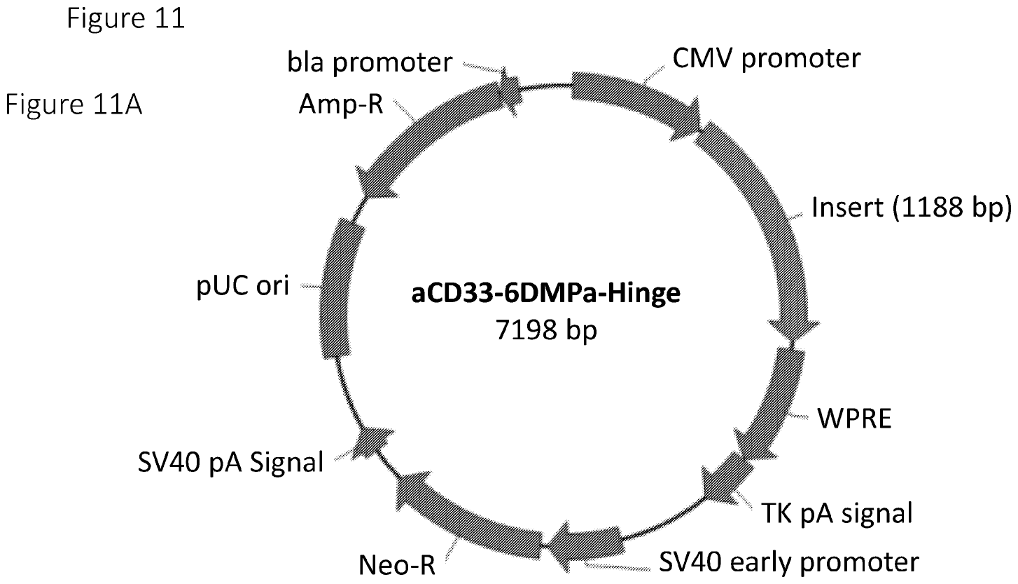

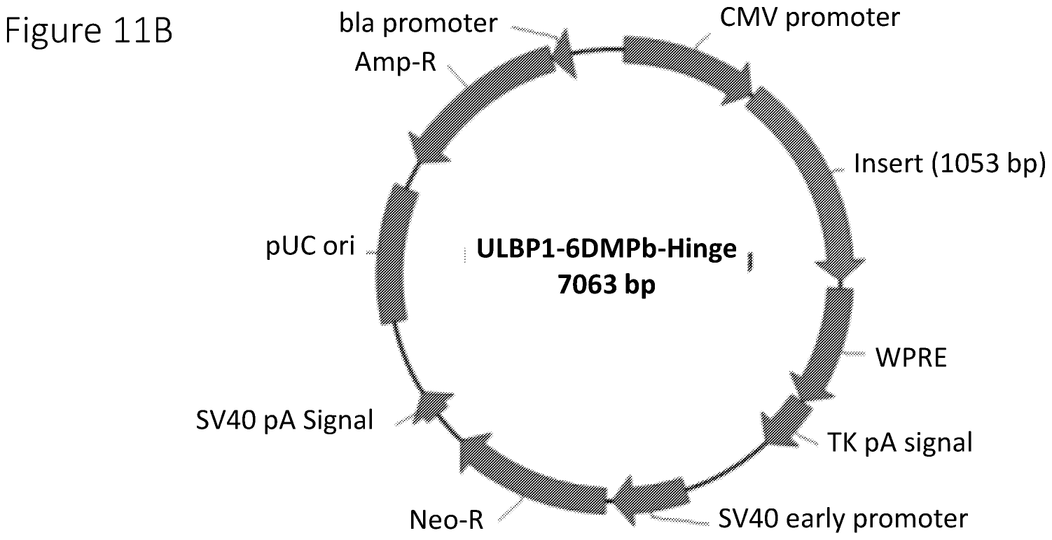

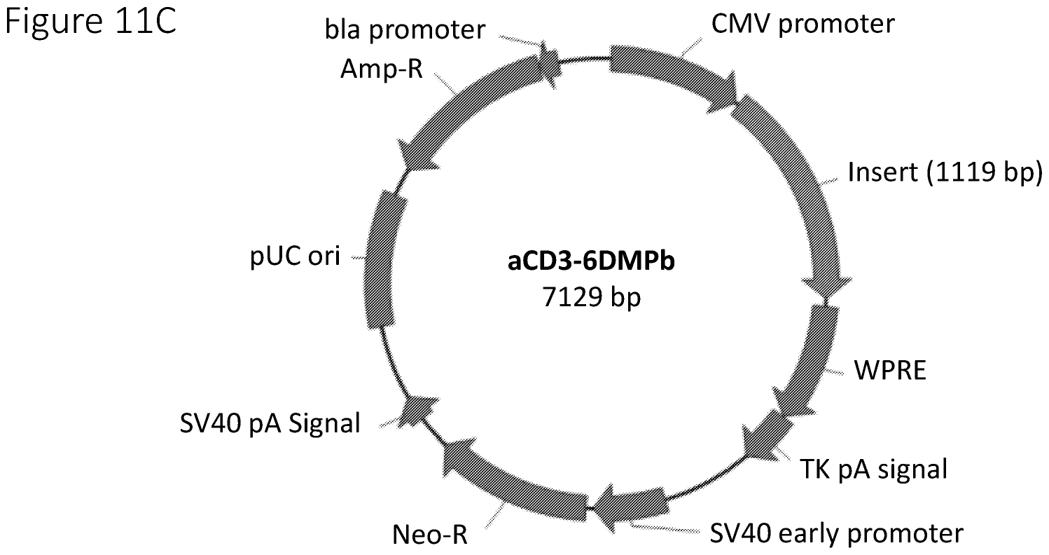

All constructs were expressed from pcDNA3.4-TOPO vectors with ampicillin resistance, codon-optimized for expression in Cricetulus griseus (Chinese hamster). See FIG. 11.

To express the polypeptides/proteins, the amino acid sequences were back translated to obtain the DNA sequences which were then codon optimized (SEQ ID NOs: 23-25).

Proteins were expressed according to a modified version of the manufacturer protocol using Lipofectamine 3000.
aCD33-6DMPa-Hinge was co-expressed in the same dish with aCD3-6DMPb-Hinge or ULBP1-6DMPb-Hinge
Proteins were produced CHO-K1 cells in 15-cm tissue-culture treated dishes 10-12×10⁶ cells were cultured the day before transfection, 37° C. 5% CO₂
   Reagent volumes per plate were as follows:
      135 uL Lipofectamine 3000
      40 ug of each plasmid (aCD33-6DMPa-Hinge and aCD3-6DMPb-Hinge)
      160 ug P3000 reagent (2 uL/ug DNA)
   Supernatant collected 3-4 days post-transfection
Cell culture supernatant was purified for His-tag containing proteins (aCD33-6DMPa-Hinge and aCD3-6DMPb-Hinge) using TALON Metal Affinity Reason (TaKaRa Bio)
   400 uL of suspended resin (200 uL of pelleted resin) used per 20 mL supernatant
      Typically, 5 plates were used at once, resulting in two 50-mL batches, each receiving 500 uL of washed and equilibrated resin
   Manufacturer protocol was followed, using the following buffers in place of HisTALON buffers:
      Wash buffer: TBST-Tris 20 mM, NaCl 150 mM, 0.1% Tween-20;
      Equilibration buffer: Tris 20 mM, NaCl 150 mM, 5 mM imidazole,
      1 mM PMSF;
      Elution buffer: NaCl 150 mM, 1 mM KH₂PO₄, 3 mM Na₂HPO₄-7H₂O, 150 mM imidazole
   Supernatant and resin rotated 30 min at 4degC before wash and elution
500 uL of dimer-bound beads were eluted 3× with Elution Buffer to a final volume ˜7.5 mL
   7.5 mL of protein-containing elution buffer was diluted to 20 mL final volume with PBS
   20 mL of protein-containing buffer was concentrated using Pierce Protein Concentrator PES (30k MWCO, 5-20 mL size), following manufacturer protocol
      Following concentration, samples once again diluted with PBS to 10⁻²⁰ mL, and concentrated once more
      30k MWCO cutoff is large enough to keep most of the dimer in upper chamber, but allows most of smaller monomers to pass to lower chamber to be discarded
Concentrated protein was quantified using Bio-Rad Protein Assay
   Samples diluted to 0.5 mg/mL with PBS, and frozen at −20C until use

Using the above protocol, a third engineered heterodimer protein is made using the constructs shown in FIGS. 3 and 23A, and FIGS. 5 and 23B.

### Example 2—Expression of the Engineered Heterodimer Proteins

The expression of anti-CD33-ULBP1 engineered heterodimer protein in 293T cells was tested using the following protocol. 293T cells mock transfected (No plasmid) or transfected with anti-CD33-ULBP1 chimeras 1 and 2 plasmids (as described in Example 1), and the cell pellets were lysed in 2×SDS gel loading buffer. The lysate were loaded on a SDS-PAGE gel and protein separated was transferred to a nylon membrane. The membrane was probed with anti-MYC antibody to detect CD33-ULBP1 and anti-Beta Actin to detect the actin protein (loading control). The membrane was also probed with a fluorochrome conjugated secondary antibody (red to detect anti-MYC; green to detect anti-beta actin) to detect the primary antibody.

As shown in FIG. 12A, the lanes loaded with lysate from the transfected cells had detectable myc protein indicative of the heterodimer protein.

Expression of the anti-CD33-ULBP1 engineered heterodimer protein in the supernatant of the 293T cells was also tested used the following protocol. Supernatant of 293T cells mock transfected (No plasmid) or transfected with anti-CD33-ULBP1 chimeras 1 and 2 plasmids were subjected to affinity purification using Talon beads. The input, flow-through, and the purified protein (Elute) was then separated on SDS-PAGE gel and transferred to a nylon membrane. The membrane was probed with ant-MYC antibody to detect. The membrane was also probed with a flurochrome conjugated secondary antibody (red to detect anti-MYC to detect the primary antibody. Again as shown in FIG. 12B, the lanes loaded with supernatant from the transfected cells had detectable myc protein indicative of the heterodimer protein.

Cell culture supernatants or eluate from TALON resin from the CHO-K1 cells transfected with chimeras as listed and described in Example 1 were added to 2×SDS loading buffer, heated, and run on an SDS Page gel. Proteins were transferred to a PVDF membrane and probed with anti-myc antibody to detect anti-CD33-6DMPa-Hinge-MycHis, and anti-FLAG antibody to detect ULBP1-6DMPb-Hinge-FLAGHis or anti-CD3-6DMPb-Hinge-FLAGHis. Fluorochrome-conjugated secondary antibodies were used to probe for the antiMyc and antiFLAG antibodies (green and red respectively).

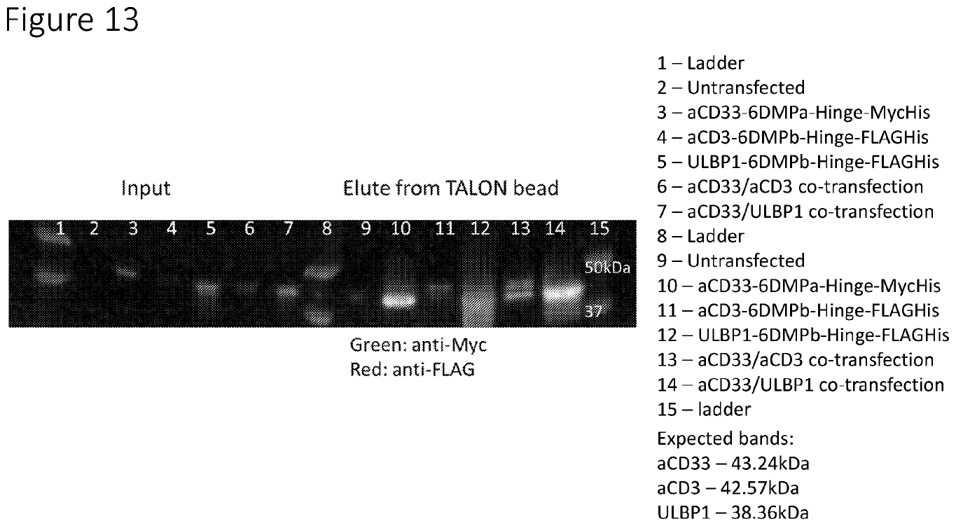

As shown in FIG. 13, the cells had detectable myc or flag protein indicative of the heterodimer proteins.

### Example 3—Binding Experiments

The binding of the engineered heterodimer proteins was tested using the following protocol:
Resuspended 50k cells in 50 uL FACS Buffer
Add 50 uL of engineered heterodimer protein, incubated 30 minutes
Wash
Resuspend in anti-MYC-FITC or anti-FLAG-PE, incubated 30 minutes

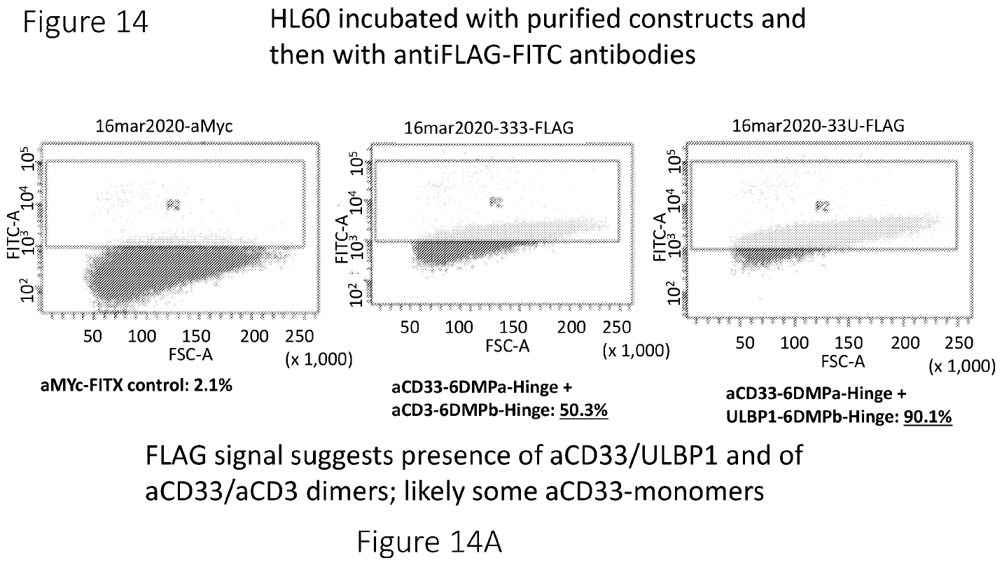

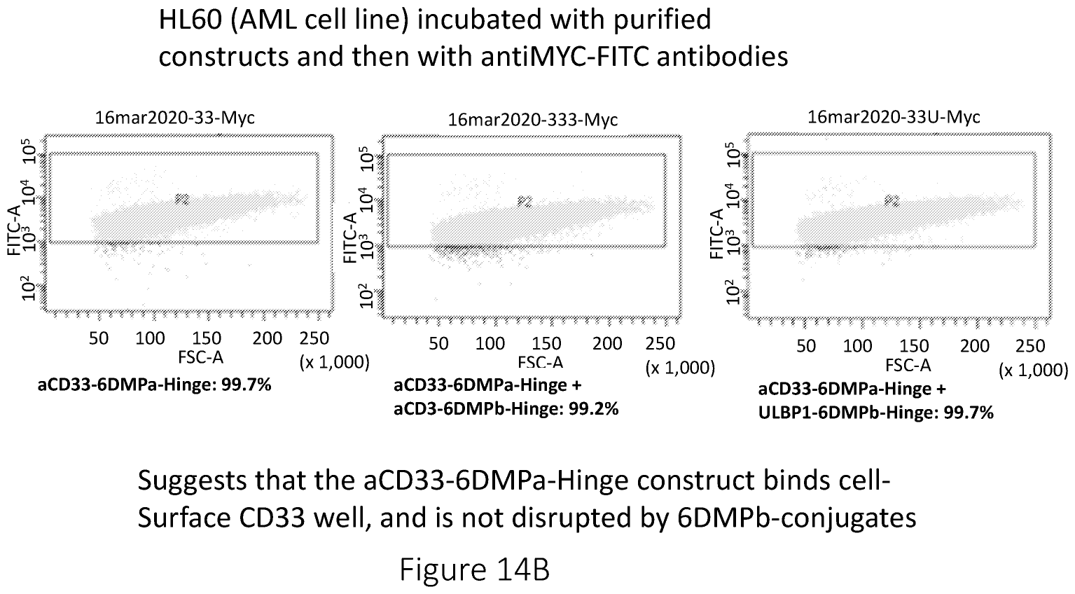

Results showed that in HL60 cells incubated with the purified engineered heterodimer proteins and then with anti-FLAG FITC antibodies there was binding of both constructs (FIG. 14A). When HL60 cells were incubated with the constructs and then anti-MYC FITC antibodies, over 99% of binding was seen for each construct as well as the CD33 construct alone, showing that the CD33 construct bound to the cell surface CD33 well and was not disrupted by the CD3 or ULBP conjugate (FIG. 14B).

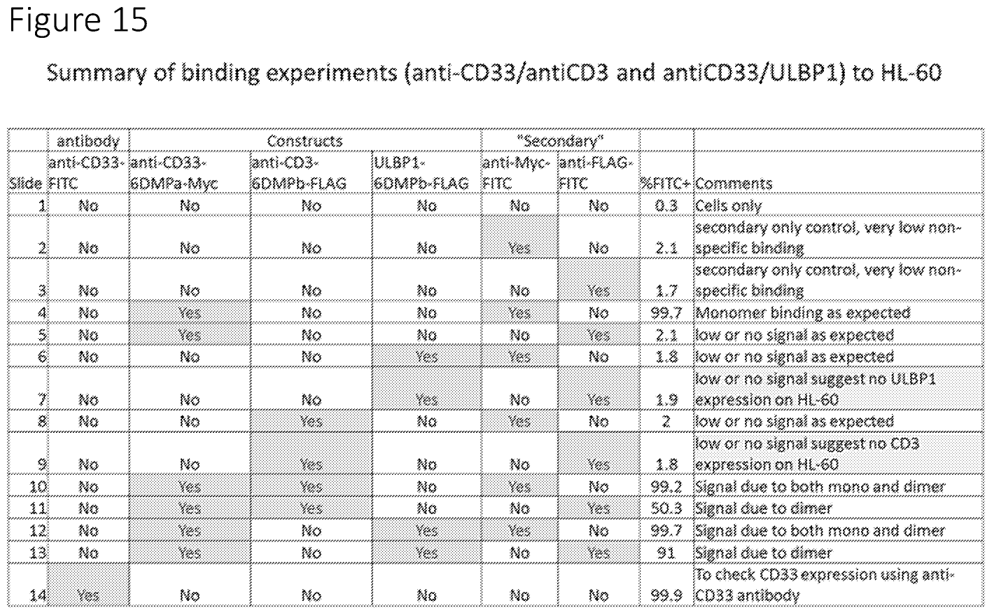

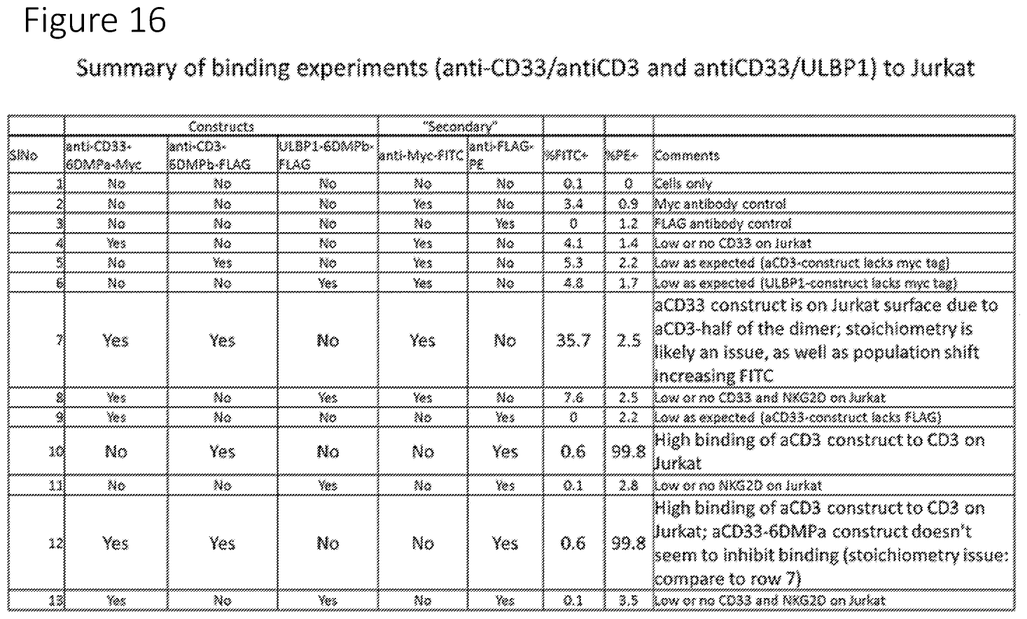

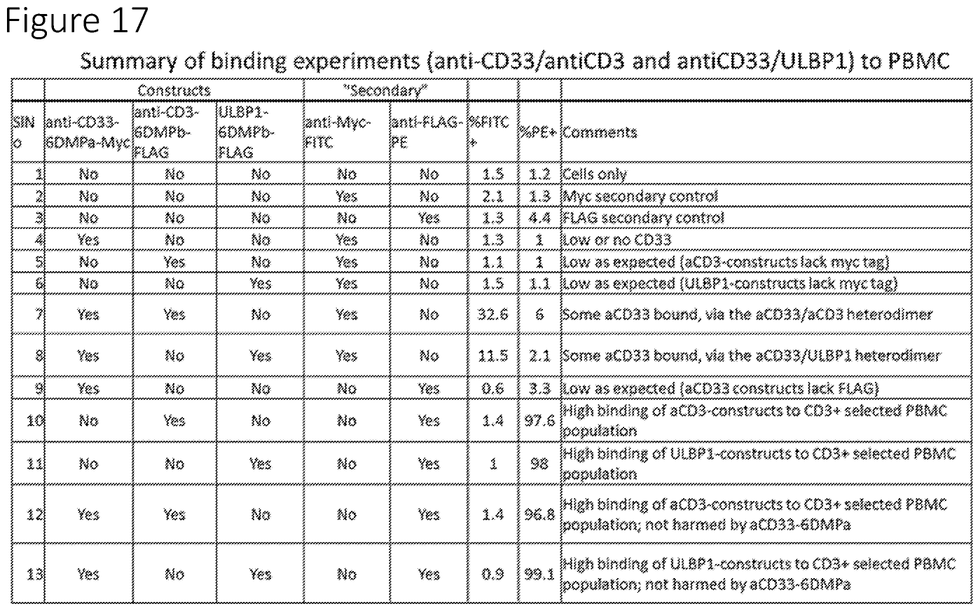

Similar results are summarized in FIGS. 15-17 for binding of the engineered proteins to HL60 cells, Jurkat cells and PMBCs.

### Example 4—Cytotoxic Activity of the Engineered Heterodimer Proteins

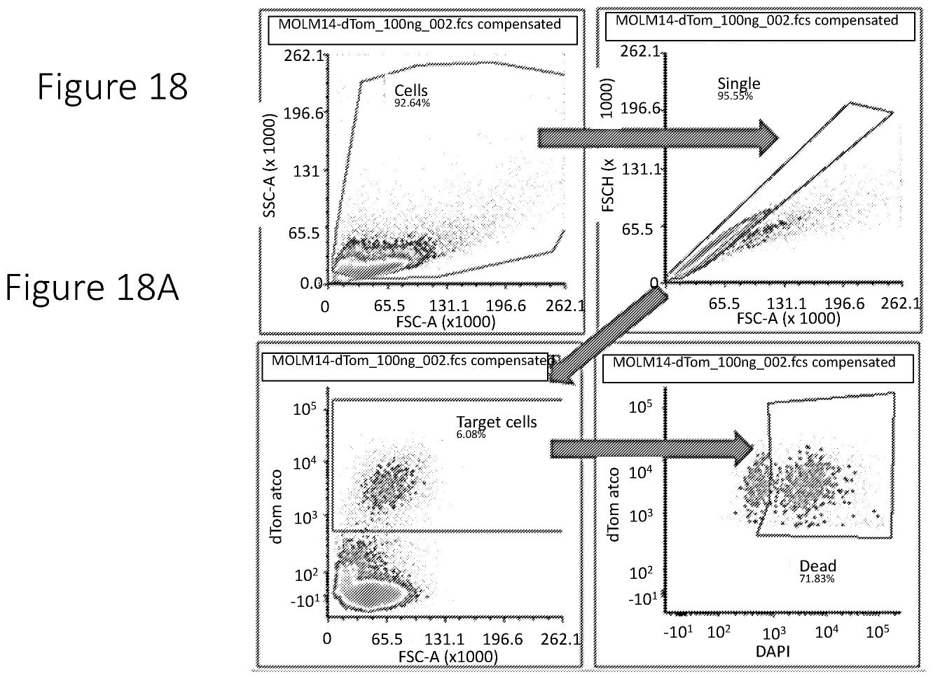

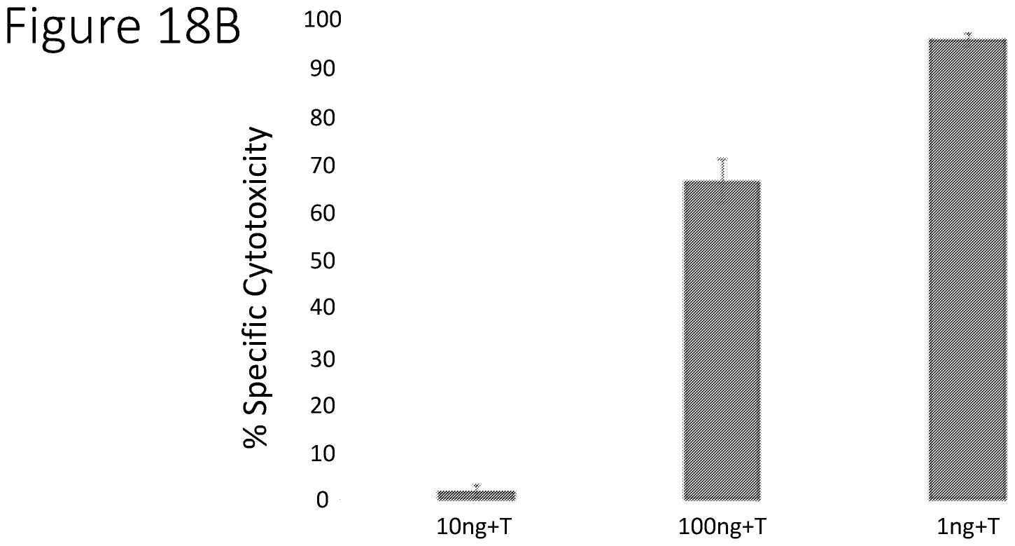

NK and CD3 in vitro cytotoxic activity of the engineered heterodimer proteins was evaluated using the following general protocol:
1. From frozen human PBMC, CD3+ cytotoxic T-cells were selected using REAlease CD3 MicroBead Kit (Miltenyi). Dynabeads Human T-activator CD3/CD28 are used immediately after to activate T-cells for one week.
2. One week after selection and activation, removed Dynabeads from T-cells.
3. Counted MOLM14-dTomato-luciferase cells (AML cell line) and T-cells (see above). Co-cultured in RPMI (+20% fetal bovine serum, 1% penicillin-streptomycin) at a ratio of 1:5 (10,000 MOLM14 cells with 50,000 T-cells).
4. Added freshly-thawed chimeric proteins to each well (e.g., various concentrations of anti-CD33-anti-CD3 engineered heterodimer)
5. Cultured overnight at 37° C., 5% CO₂.
6. Spun plates (500 rpm, 5 min), remove supernatant.
7. Washed with 200 uL FACS buffer (PBS with: 1% FBS, 1 mM EDTA)
8. Spun plates (500 rpm, 5 min), remove supernatant
9. Resuspended in 100 uL FACS buffer, add DAPI to final concentration of 0.1 ug/mL
10. Proceeded to FACS analysis; gate for single cells, and identify target cells as PE+DAPI+ (dTomato in MOLM14 AML cells in PE channel, dead cells in DAPI channel) See FIG. 18A.
11. Compared % of DAPI+ cells among PE+ cells in the T-cell+dimer group, considering wells with MOLM14 and T-cells (without dimer) as baseline. % cell death was normalized to control group with T-cells and MOLM14 cells co-incubated without dimer treatment according to the formula below:

[(death from T-cells and dimer)−(death from T-cells alone)]/[100−(death from T-cells alone)]*100

In one experiment, MOLM14-dTomato cells were incubated with varying amount of anti-CD33-anti-CD3 heterodimer (10 ng, 100 ng, 1 μg and 10 μg) with 5:1 ratio of T-cells (effector) as well as control (no heterodimer, no T cells) and incubation with 10 μg of heterodimer only and 5:1 T cells only for 24 hours.

As shown in FIG. 18B, cytotoxicity was seen for all of the doses of heterodimer with the T cells (effector cells) as compared to the controls, dimer alone or T cells alone and anti-CD33-anti-CD3 heterodimer enhanced the killing of CD33-expressing target cells (MOLM14) by effector T-cells in a dose dependent manner.

In a further experiment using the protocol above, activated T-cells (CD3+) selected from PBMCs were co-incubated with CellTrace Violet CD33 expressing HL-60 target cells for 16 hours, in the presence of anti-CD33-anti-CD3 heterodimer at three concentrations.

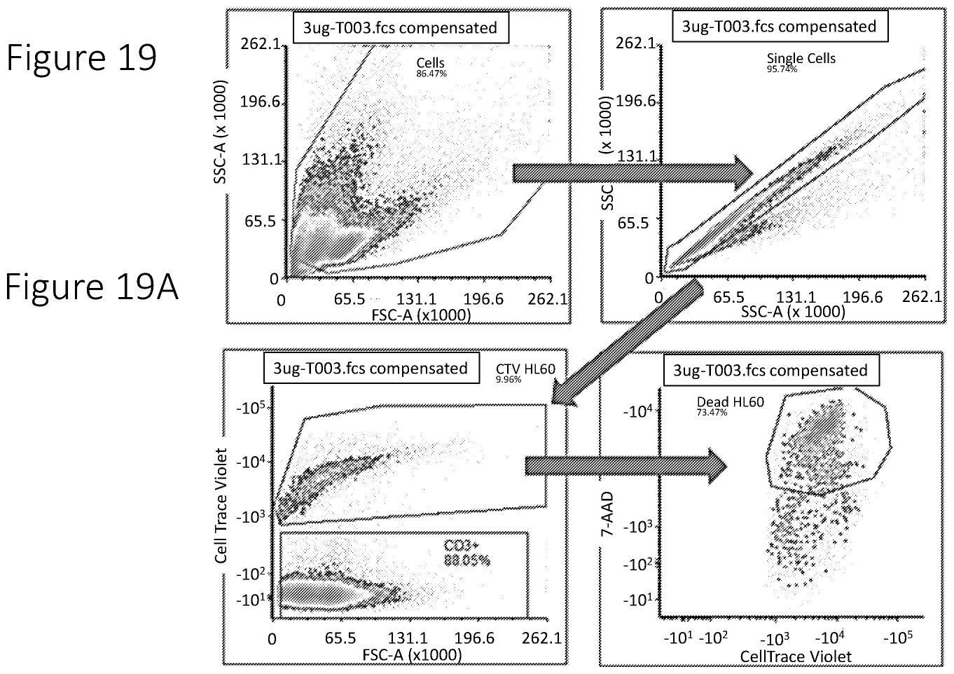

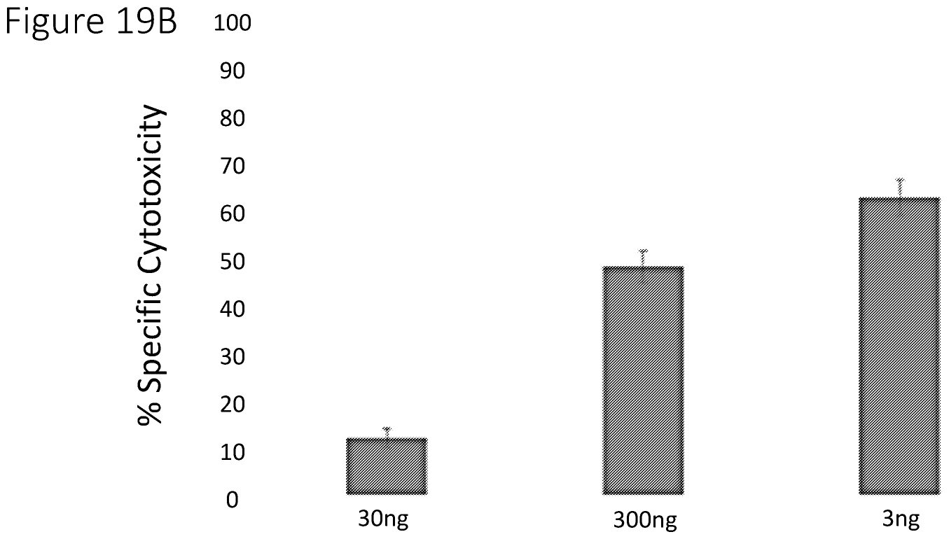

Following incubation, cells were stained with 7-AAD viability dye and analyzed using flow cytometry. See FIG. 19A. Percent (%) cell death was normalized to control group with T-cells and HL-60 cells co-incubated without dimer treatment according to the formula below:

[(death from T-cells and dimer)−(death from T-cells alone)]/[100−(death from T-cells alone)]*100

As shown in FIG. 19B, anti-CD33-anti-CD3 heterodimer enhances killing of CD33-expressing target cells (HL60) by effector T-cells in a dose dependent manner.

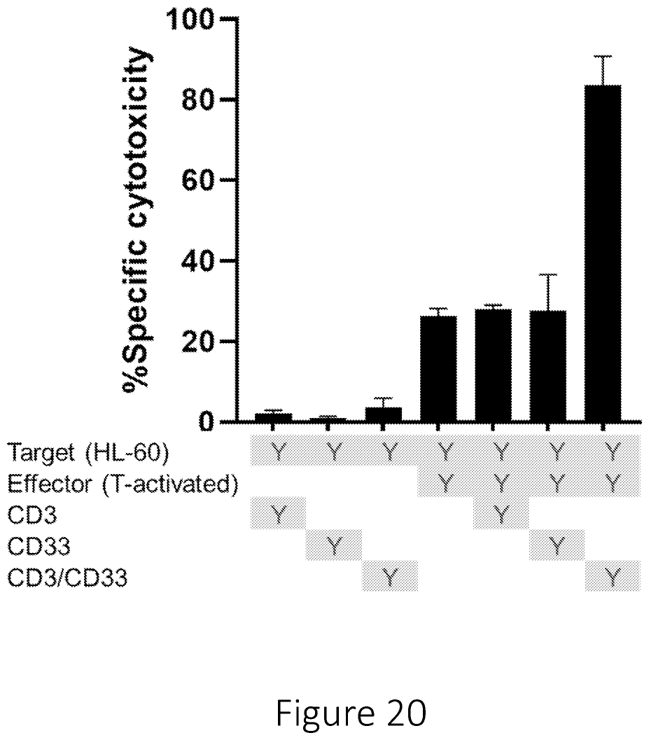

An additional cytotoxicity assay was performed as follows: AML cells (HL-60) were co-incubated with either monomer of CD3 or CD33 or with anti-CD33-anti-CD33 heterodimer with and without T-cells for 24 hours and viability was measured using live/dead staining using 7AAD and flow cytometry as described. Results in FIG. 20 show that the heterodimers but not the monomers enhanced effector cells (T-cells) cytotoxic activity on AML cells.

A further dose dependent cytotoxicity assay was performed as follows: AML cells (HL-60) were co-incubated with anti-CD3-anti-CD33 heterodimer at the indicated amounts (3, 30, 300 or 30000 ng of heterodimer) with or without T-cells for 24 hours and viability was measured using live/dead staining using 7AAD and flow cytometry as described.

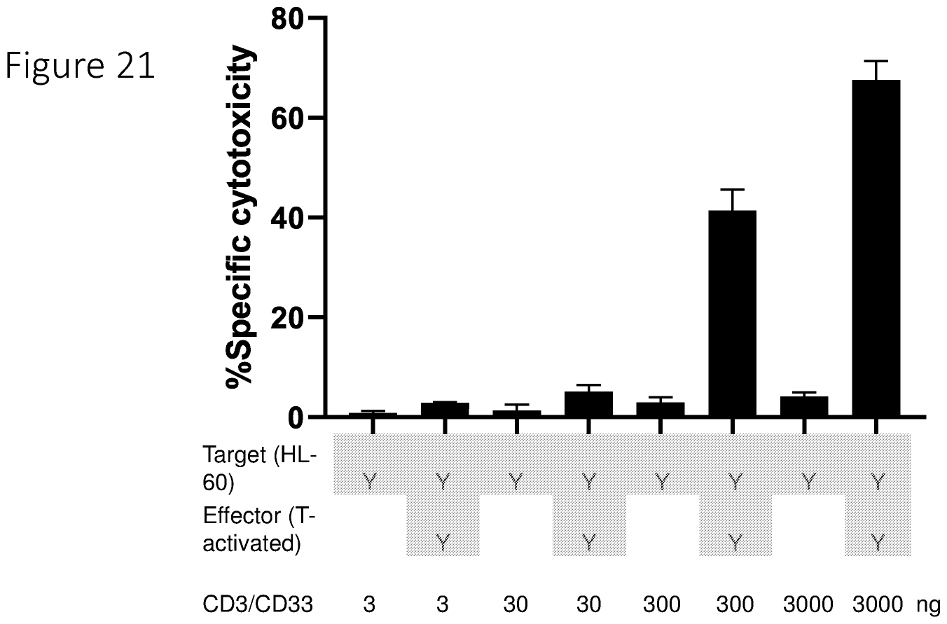

Dose dependent enhancement of effector cell cytotoxic activity of the heterodimer on AML cells was seen. There was no appreciable increase in cytotoxicity with the anti-CD33-anti-CD3 heterodimer alone (no T-cells) between 3 and 3000 ng, suggesting the anti-CD33-anti-CD3 heterodimer does not show cytotoxic by itself and the cytotoxic effects are mediated via enhancement of T-cell function. See FIG. 21.

Another dose dependent cytotoxicity assay was performed as follows: AML cells (HL-60) were incubated with 300 ng of anti-CD33-anti-CD3 heterodimer with varying ratios of T-cells (effector) (1:1, 2:1, 5:1, and 10:1 effector to target) for 24 hours and viability was measured using live/dead staining using 7AAD and flow cytometry as described.

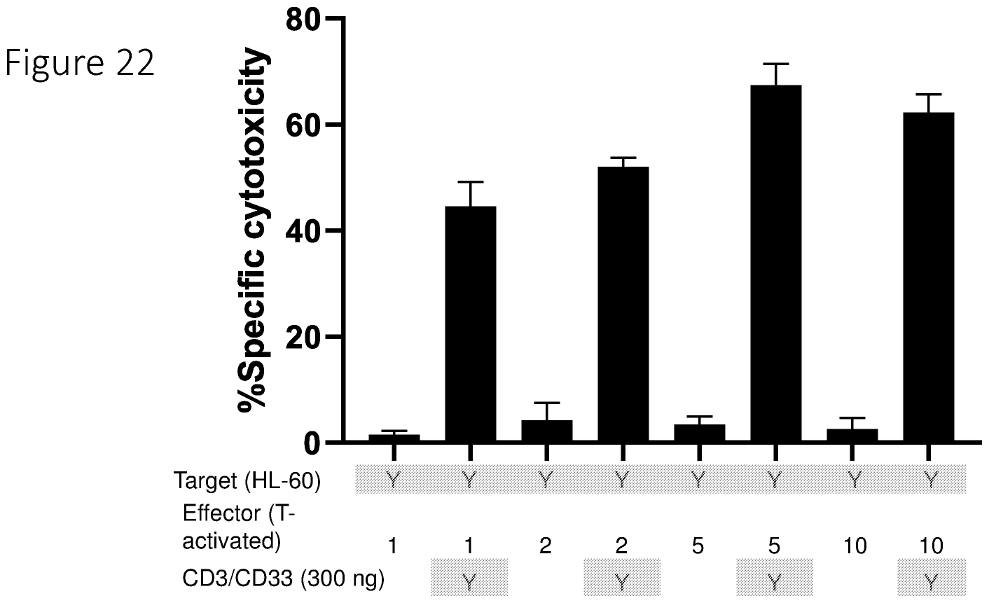

A dose dependent increase in cytotoxicity was seen up to the 5:1 ratio of effector to target using 300 ng of heterodimer. There was no appreciable increase in cytotoxicity between the ratios of 5:1 and 10:1 effector to target, likely because a maximal cytotoxicity was reached. See FIG. 22

All the above data suggest that heterodimer was functionally active and enhanced the T-cell cytotoxic function towards the AML cells. The data also shows that the cytotoxicity of the heterodimer is increase in cells which exhibit a higher expression of CD33, such as MOLM14 cells. See FIG. 18B versus FIG. 19B.

### Example 5—In Vivo Anti-Tumor Activity of the Engineered Heterodimer Proteins

The in vivo activity of the anti-CD33-anti-CD3 engineered heterodimer protein was tested in mice using the following protocol. All mice received 2×10⁵ MOLM14-dTomato-luciferase cells (AML cell line), then at day 2 and following the below schedule, one group received no treatment, one group received unloaded T cells (“unloaded T cells”) and one group T cells loaded with the anti-CD33-anti-CD3 engineered heterodimer protein (Bite) (“loaded T cells”).

Day 2: 10 million T cells with or without 100 ugr anti-CD33-anti-CD3 engineered heterodimer protein;

Days 6 and 11: 20 million T cells with or without 200 ugr anti-CD33-anti-CD3 engineered heterodimer protein

Days 15 and 20: 40 million T cells with or without 400 ugr anti-CD33-anti-CD3 engineered heterodimer protein.

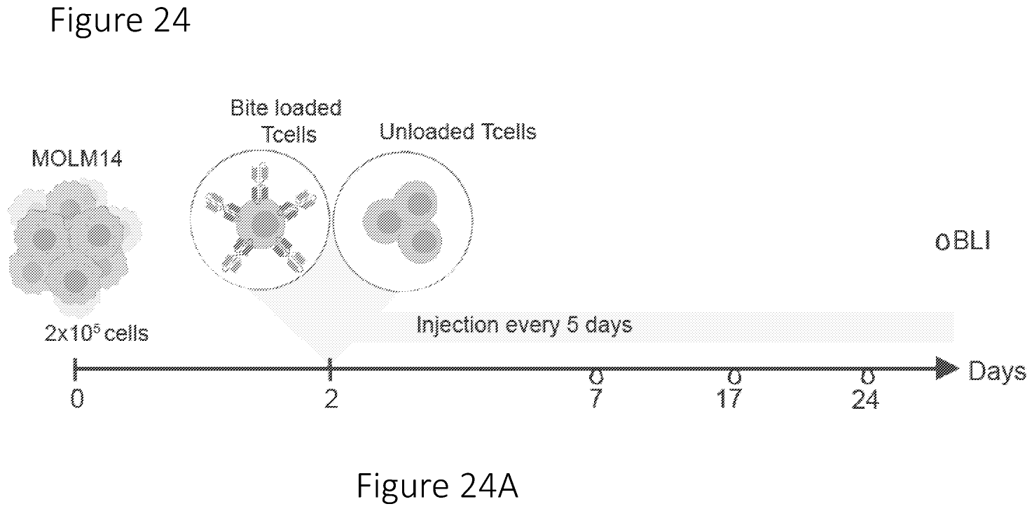

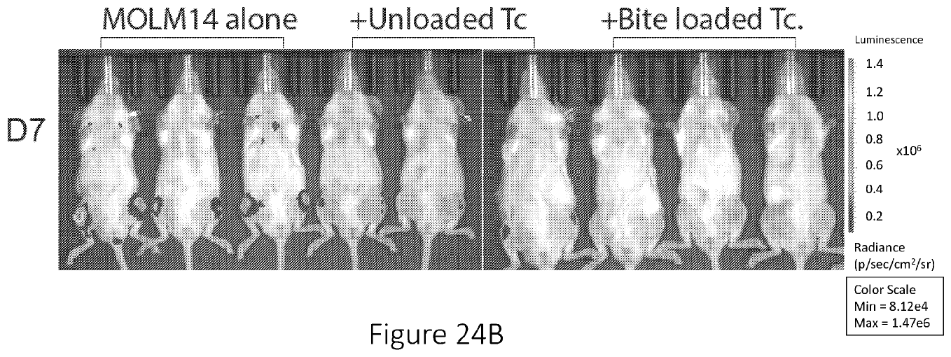

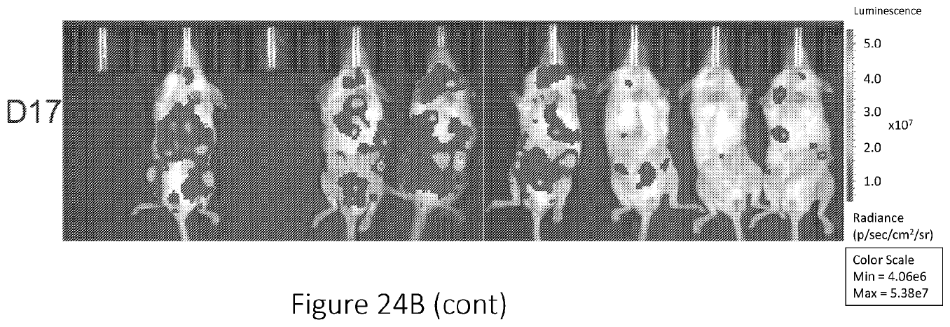

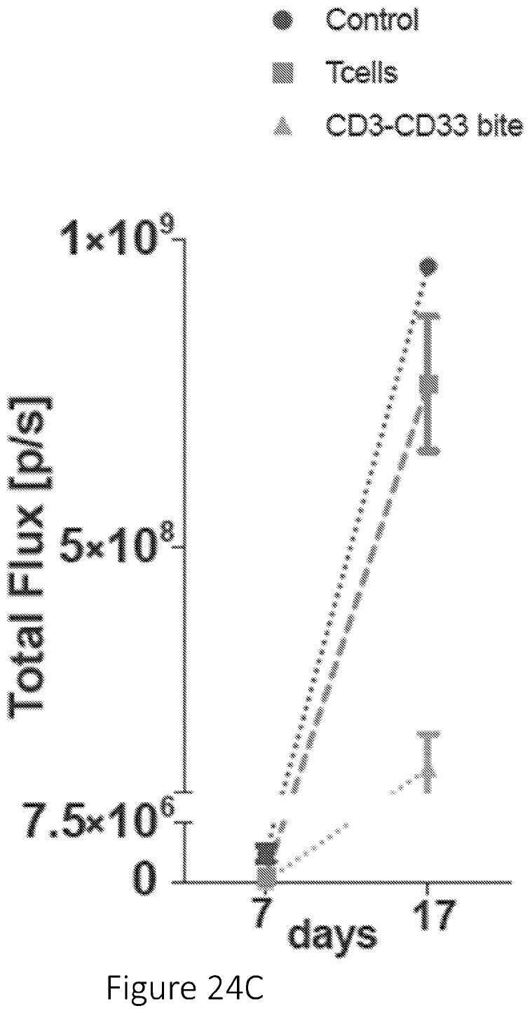

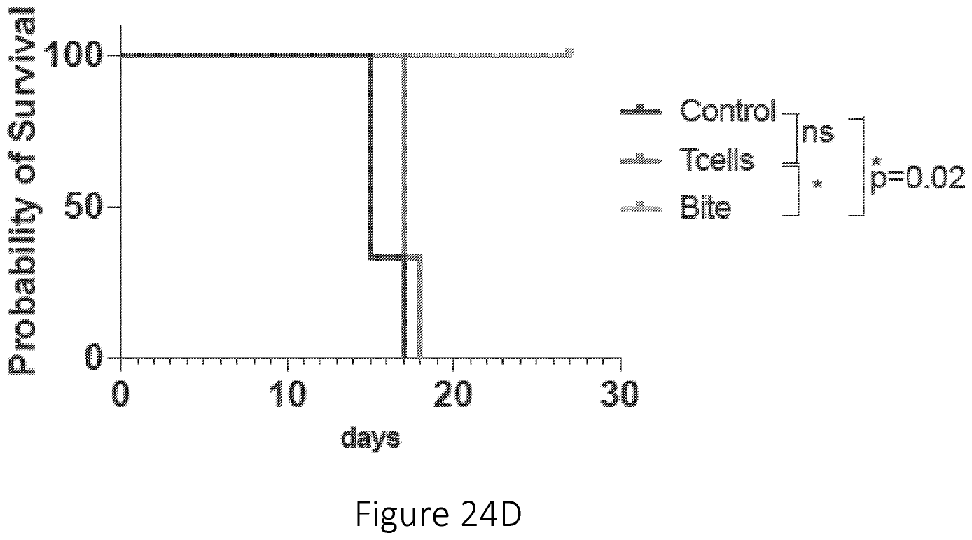

Bioluminescence imaging (BLI) to monitor the growth of FFluc-dtomato transduced MOLM14 on days 7, 17 and 24. See FIG. 24A.

Mice were sacked for analysis when one or more leukemia-related symptoms were observed such as hunch-backed, significant weight loss, ruffled coat, and limb paralysis.

As shown in FIGS. 24B and C, mice treated with loaded T cells had lower tumor burden at days 7 and 17.

Additionally, mice treated with the loaded T cells had better survival than the 2 control groups (no treatment or unloaded T cells). The Kaplan-Meier survival plot was made with starting date as the date of MOLM14 injection and end date as date of death/sacking for each mouse. See FIG. 24D.

### REFERENCES

Benci, et al. “Tumor Interferon Signaling Regulates a Multigenic Resistance Program to Immune Checkpoint Blockade.” Cell. 167(6):1540-54. 2016.
Chamuleau, et al. “High INDO (indoleamine 2,3-dioxygenase) mRNA level in blasts of acute myeloid leukemic patients predicts poor clinical outcome.” Haematologica. 93(12):1894-8. 2008.
Chen, et al. “Programmable design of orthogonal protein heterodimers.” Nature 565: 106-111. 2019.
Dohner et al., NEJM (2015) 373:1136.
Dulphy, et al. A. “Underground adaptation to a hostile environment: acute myeloid leukemia vs. natural killer cells.” Front Immunol. 7(94):1-15. 2016.
Elias, et al. “Immune evasion by oncogenic proteins of acute myeloid leukemia.” Blood 123(10):1535-43. 2014.
Krupka, et al. “CD33 target validation and sustained depletion of AML blasts in long-term cultures by the bi-specific T-cell-engaging antibody AMG 330.” Blood. 123:356-365. 2014.
Kufer, et al. “Pharmaceutical compositions comprising bispecific anti-CD3, anti-CD19 antibody constructs for the treatment of B-cell related disorders.” Patent CA2522586. 2017.
Le Dieu, et al. “Peripheral blood T cells in acute myeloid leukemia (AML) patients at diagnosis have abnormal phenotype and genotype and form defective immune synapses with AML blasts.” Blood 114(18):3909-16. 2009.
Löwenberg, et al. Dutch-Belgian Hemato-Oncology Cooperative Group (HOVON)., German Austrian AML Study Group (AMLSG)., Swiss Group for Clinical Cancer Research Collaborative Group (SAKK). “Gemtuzumab ozogamicin as postremission treatment in AML at 60 years of age or more: results of a multicenter phase 3 study.” Blood. 115(13):2586-91. 2010.
Mastaglio, et al. “Natural killer receptor ligand expression on acute myeloid leukemia impacts survival and relapse after chemotherapy”. Blood Adv. 2(4):335-346. 2018.
Mundy-Bosse, et al. “Acute Myeloid Leukemia Alters Natural Killer Cell Maturation and Functional Activation.” Blood. 124(21):754. 2014.
Nanbakhsh, et al. “c-Myc regulates expression of NKG2D ligands ULBP1/2/3 in AML and modulates their susceptibility to NK-mediated lysis”. Immunobio. 123(23):3585-3596. 2014.
Orleans-Lindsay, et al. “Acute myeloid leukaemia cells secrete a soluble factor that inhibits T1 and NK cell proliferation but not cytolytic function—implications for the adoptive immunotherapy of leukaemia.” Clin Exp Immunol. 126(3):403-11. 2001.
Renneville, et al. “Clinical impact of gene mutations and lesions detected by SNP-array karyotyping in acute myeloid leukemia patients in the context of gemtuzumab ozogamicin treatment: results of the ALFA-0701 trial.” Oncotarget. 5(4):916-932. 2014.
Schnorfeil, et al. “T cells are functionally not impaired in AML: increased PD-1 expression is only seen at time of relapse and correlates with a shift towards the memory T cell compartment.” J Hematol Oncol. 8:93. 2015.
Shenghui, et al. “Elevated frequencies of CD4(+) CD25(+) CD127lo regulatory T cells is associated to poor prognosis in patients with acute myeloid leukemia.” Int J Cancer. 129(6):1373-81. 2011.
Stringaris, et al. “Leukemia-induced phenotypic and functional defects in natural killer cells predict failure to achieve remission in acute myeloid leukemia.” Haematologica. 99(5):836-47. 2014.
Thorsson, et al. “The Immune Landscape of Cancer.” Immunity. 48(4):812-30. 2018.
Wypych., et al. “Human IgG2 Antibodies Display Disulfide-mediated Structural Isoforms.” J Biol Chem. 283:16194-205. 2008.

1. An engineered heterodimer protein, comprising:
(a) a first polypeptide comprising an antigen-binding fragment that binds a lineage-specific cell-surface antigen, a non-naturally occurring polypeptide domain comprising 1-5 alpha helices connected by amino acid linkers and a first covalent dimerization domain; and
(b) a second polypeptide comprising a polypeptide that binds a molecule expressed on natural killer (NK) cells or a molecule expressed on T cells, a non-naturally occurring polypeptide domain comprising 1-5 alpha helices connected by amino acid linkers, and a second covalent dimerization domain; and wherein the first and second polypeptides are covalently bonded through the covalent dimerization domain.

2. The engineered heterodimer protein of claim 1, wherein the molecule expressed on NK cells is NKG2D, and wherein the polypeptide that binds a molecule expressed on NK cells is ULBP1, ULBP2, ULBP3, ULBP4, ULBP5, ULBP6, MICA, MICB, or mutants or fragments thereof.

3. The engineered heterodimer protein of claim 2, wherein the polypeptide that binds a molecule expressed on NK cells is an ectodomain of ULBP1, ULBP2, ULBP3, ULBP4, ULBP5, ULBP6, MICA, or MICB.

4. The engineered heterodimer protein of claim 1, wherein the molecule expressed on NK cells is CD16, and wherein the polypeptide that binds a molecule expressed on T cells is a mono clonal antibody of CD 16.

5. (canceled)

6. The engineered heterodimer protein of claim 1, wherein the molecule expressed on T cells is CD3, and wherein the polypeptide that binds a molecule expressed on T cells is a mono clonal antibody of CD3.

7. The engineered heterodimer protein of claim 1, wherein the first dimerization domain and/or the second dimerization domain comprise an IgG2 hinge domain.

8. The engineered heterodimer protein of claim 7, wherein the first dimerization domain and/or the second dimerization domain further comprise an IgG2 Fc domain.

9. The engineered heterodimer protein of claim 1, wherein the non-naturally occurring polypeptide domain comprising 1-5 alpha helices connected by amino acid linkers in the first polypeptide and the second polypeptide comprise 6DMPa and 6DMPb, respectively.

10. The engineered heterodimer protein of claim 1, wherein the non-naturally occurring polypeptide domain comprising 1-5 alpha helices connected by amino acid linkers in the first polypeptide and the second polypeptide comprise 6DMPb and 6DMPa, respectively.

11. The engineered heterodimer protein of claim 1, wherein the lineage-specific cell-surface antigen is CD33.

12. The engineered heterodimer protein of claim 1, wherein the antigen-binding fragment is a single-chain antibody fragment (scFv).

13. A composition comprising at least one vector encoding the engineered heterodimer protein of claim 1.

14. A kit comprising the composition of claim 13.

15. A method of treating a hematopoietic malignancy in a subject, comprising administering to the subject an effective amount of the composition of claim 13.

16. An engineered heterotrimer protein, comprising:
(a) a first polypeptide comprising a polypeptide that binds a molecule expressed on T cells, a non-naturally occurring polypeptide domain comprising 1-5 alpha helices connected by amino acid linkers (a1), and a first covalent dimerization domain;
(b) a second polypeptide comprising an antigen-binding fragment that binds a lineage-specific cell-surface antigen, a non-naturally occurring polypeptide domain comprising 1-5 alpha helices connected by amino acid linkers (b1), and a second covalent dimerization domain;
(c) a third polypeptide comprising a polypeptide that binds a molecule expressed on natural killer (NK) cells, a non-naturally occurring polypeptide domain comprising 1-5 alpha helices connected by amino acid linkers (c1), and a third covalent dimerization domain; and
(d) a fourth polypeptide comprising three non-naturally occurring polypeptide domains comprising 1-5 alpha helices connected by amino acid linkers, wherein each domain is the binding domain of a1, b1 and c1 (a2, b2 and c2), and a fourth, fifth and sixth covalent dimerization domain; wherein the first and second and third and fourth polypeptides are covalently bonded through the covalent dimerization domain.

17. The engineered heterotrimer protein of claim 16, wherein the molecule expressed on T cells is CD3, and wherein the polypeptide that binds a molecule expressed on T cells is a mono clonal antibody of CD3, wherein the lineage-specific cell-surface antigen is CD33, and wherein the molecule expressed on NK cells is NKG2D.

18. The engineered heterotrimer protein of claim 16, wherein the molecule expressed on T cells is CD3, and wherein the polypeptide that binds a molecule expressed on T cells is a mono clonal antibody of CD3, wherein the lineage-specific cell-surface antigen is CD33, and wherein the molecule expressed on NK cells is CD 16, and wherein the polypeptide that binds a molecule expressed on NK cells is a monoclonal antibody of CD 16.

19. The engineered heterotrimer protein of claim 16, wherein the first dimerization domain and/or the second dimerization domain and/or the third dimerization domain and/or the fourth dimerization domain and/or the fifth dimerization domain and/or the sixth dimerization domain comprise an IgG2 hinge domain.

20. The engineered heterotrimer protein of claim 19, wherein the first dimerization domain and/or the second dimerization domain and/or the third dimerization domain and/or the fourth dimerization domain and/or the fifth dimerization domain and/or the sixth dimerization domain further comprise an IgG2 Fc domain.

21. The engineered heterotrimer protein of claim 16, wherein the non-naturally occurring polypeptide domain comprising 1-5 alpha helices connected by amino acid linkers in the first polypeptide and the second polypeptide and the third polypeptide and the fourth polypeptide are chosen from the group consisting of 6DMPa and 6DMPb.

22. A composition comprising at least one vector encoding the engineered heterotrimer protein of claim 16.

23. A kit comprising the composition of claim 22.

24. A method of treating a hematopoietic malignancy in a subject, comprising administering to the subject an effective amount of the composition of claim 22.


SEQUENCE LISTING (30 sequences)

SEQ ID NO: 1 - protein, 393 aa, Artificial Sequence
  Engineered Protein
MYRMQLLSCIALSLALVTNSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSSSAGGGGSGGGGSGGGGSGTKEDILERQRKIIERAQEIHRRQQEILEELERIIRKPGSSEEAMKRMLKLLEESLRLLKELLELSEESAQLLYEQRERKCCVECPPCPEQKLISEEDLHHHHHH

SEQ ID NO: 2 - protein, 333 aa, Artificial Sequence
  Engineered Protein
MYRMQLLSCIALSLALVTNSWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGGGGGSGGGGSGGGGSTEKRLLEEAERAHREQKEIIKKAQELHRRLEEIVRQSGSSEEAKKEAKKILEEIRELSKRSLELLREILYLSQEQKGSLVPRERKCCVECPPCPDYKDDDDKHHHHHH

SEQ ID NO: 3 - protein, 388 aa, Artificial Sequence
  Engineered Protein
MYRMQLLSCIALSLALVTNSDIKLQQSGAELARPGASVKMSCKTSGYTFTRYTMHWVKQRPGQGLGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGGKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSTEKRLLEEAERAHREQKEIIKKAQELHRRLEEIVRQSGSSEEAKKEAKKILEEIRELSKRSLELLREILYLSQEQKGSLVPRERKCCVECPPCPDYKDDDDKHHHHHH

SEQ ID NO: 4 - protein, 609 aa, Artificial Sequence
  Engineered Protein
MYRMQLLSCIALSLALVTNSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSSSAGGGGSGGGGSGGGGSGTKEDILERQRKIIERAQEIHRRQQEILEELERIIRKPGSSEEAMKRMLKLLEESLRLLKELLELSEESAQLLYEQRERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKEQKLISEEDLHHHHHH

SEQ ID NO: 5 - protein, 549 aa, Artificial Sequence
  Engineered Protein
MYRMQLLSCIALSLALVTNSWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGGGGGSGGGGSGGGGSTEKRLLEEAERAHREQKEIIKKAQELHRRLEEIVRQSGSSEEAKKEAKKILEEIRELSKRSLELLREILYLSQEQKGSLVPRERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDYKDDDDKHHHHHH

SEQ ID NO: 6 - protein, 604 aa, Artificial Sequence
  Engineered protein
MYRMQLLSCIALSLALVTNSDIKLQQSGAELARPGASVKMSCKTSGYTFTRYTMHWVKQRPGQGLGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGGKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSTEKRLLEEAERAHREQKEIIKKAQELHRRLEEIVRQSGSSEEAKKEAKKILEEIRELSKRSLELLREILYLSQEQKGSLVPRERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDYKDDDDKHHHHHH

SEQ ID NO: 7 - protein, 20 aa, Artificial Sequence
  Synthetic polypeptide
MYRMQLLSCIALSLALVTNS

SEQ ID NO: 8 - protein, 10 aa, Artificial Sequence
  Synthetic polypeptide
EQKLISEEDL

SEQ ID NO: 9 - protein, 113 aa, Artificial Sequence
  Synthetic polypeptide
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 10 - protein, 122 aa, Artificial Sequence
  Synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSSSA

SEQ ID NO: 11 - protein, 15 aa, Artificial Sequence
  Synthetic polypeptide
GGGGSGGGGSGGGGS

SEQ ID NO: 12 - protein, 77 aa, Artificial Sequence
  Synthetic polypeptide
GTKEDILERQRKIIERAQEIHRRQQEILEELERIIRKPGSSEEAMKRMLKLLEESLRLLKELLELSEESAQLLYEQR

SEQ ID NO: 13 - protein, 12 aa, Artificial Sequence
  Synthetic polypeptide
ERKCCVECPPCP

SEQ ID NO: 14 - protein, 8 aa, Artificial Sequence
  Synthetic polypeptide
DYKDDDDK

SEQ ID NO: 15 - protein, 190 aa, Artificial Sequence
  Synthetic polypeptide
WVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 16 - protein, 82 aa, Artificial Sequence
  Synthetic polypeptide
TEKRLLEEAERAHREQKEIIKKAQELHRRLEEIVRQSGSSEEAKKEAKKILEEIRELSKRSLELLREILYLSQEQKGSLVPR

SEQ ID NO: 17 - protein, 121 aa, Artificial Sequence
  Synthetic polypeptide
DIKLQQSGAELARPGASVKMSCKTSGYTFTRYTMHWVKQRPGQGLGLEWIGYINPSRGYTNYNQKFKDKATLTTDKSSSTAYMQLSSLTSEDSAVYYCARYYDDHYCLDYWGQGTTLTVSS

SEQ ID NO: 18 - protein, 100 aa, Artificial Sequence
  Synthetic polypeptide
DIQLTQSPAIMSASPGGKVTMTCRASSSVSYMNWYQQKSGTSPKRWIYDTSKVASGVPYRFTSYSLTISSMEAEDAATYYCQQWSSNPLTFGAGTKLELK

SEQ ID NO: 19 - protein, 216 aa, Artificial Sequence
  Synthetic polypeptide
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 20 - protein, 6 aa, Artificial Sequence
  Synthetic polypeptide
HHHHHH

SEQ ID NO: 21 - protein, 14 aa, Artificial Sequence
  Synthetic polypeptide
GGGGGSGGSGGSGG

SEQ ID NO: 22 - protein, 6 aa, Artificial Sequence
  Synthetic polypeptide
SGSGSG

SEQ ID NO: 23 - DNA, 1188 bp, Artificial Sequence
  Synthetic polynucleotide
gccccatgtacagaatgcagctgctgagctgtatcgccctgtctctggccctggtcaccaactctgagatcgtgctgacacagagccctggcagcctggctgtttctcctggcgagcgagtgaccatgagctgcaagtctagccagagcgtgttcttcagcagcagccagaagaactacctggcctggtatcagcagatccccggacagtctccccggctgctgatctattgggccagcacaagagaaagcggcgtgcccgatagattcacaggctctggcagcggcaccgacttcaccctgacaatcagtagcgtgcagcccgaggacctggccatctactactgtcaccagtacctgagcagccggacctttggccagggcaccaagctggaaatcaagagaggcagcacaagcggcagcggaaagcctggatctggcgagggctctacaaaaggccaggttcagctgcaacagcctggcgccgaagttgtgaaacctggcgcctctgtgaagatgtcctgcaaggccagcggctacaccttcaccagctactacatccactggatcaagcagaccccaggccaaggcctggaatgggtcggagtgatctaccccggcaacgacgacatcagctacaaccagaagttccagggcaaagccacactgaccgccgacaagtccagcaccacagcctacatgcagctgtccagcctgaccagcgaagatagcgccgtgtactactgcgccagagaagtgcggctgcggtactttgatgtgtggggccagggaaccaccgtgaccgtttctagctcatcttctgctggcggcggaggaagcggaggcggaggttctggtggtggtggctctggaacaaaagaggacatcctggaacggcagaggaagatcatcgagcgggcccaagagatccacagacggcagcaagagattctggaagaactggaacggatcatcagaaagcccggcagcagcgaagaggccatgaagagaatgctgaagctgctggaagagagcctgagactgctgaaagaactgctggaactgagcgaggaaagcgcccagctgctgtacgagcagagggaaagaaagtgctgcgtggaatgccctccttgtcctgagcagaagctgatctccgaagaggacctgcachcaccatcaccaccactga

SEQ ID NO: 24 - DNA, 1053 bp, Artificial Sequence
  Synthetic polynucleotide
gccaccatgtacagaatgcagctgctgagctgtatcgccctgtctctggccctggtcaccaatagctgggtcgacacccactgcctgtgctacgacttcatcatcacccctaagagcagacccgagcctcagtggtgcgaagtgcaaggactggtggacgagaggcccttcctgcactacgattgcgtgaaccacaaggccaaggcctttgccagcctgggcaagaaagtgaacgtgaccaagacctgggaagaacagaccgagacactgcgcgacgtggtggattttctgaagggccagctgctggacatccaggtggaaaatctgatccccatcgagcccctgacactgcaggccagaatgtcttgtgaacacgaggcccacggccacggcagaggatcttggcagttcctgttcaacggccagaagttcctgctgttcgacagcaacaaccggaagtggaccgctctgcatcctggcgccaagaaaatgaccgagaagtgggagaagaaccgggacgtgaccatgttcttccagaagatctccctgggcgactgcaagatgtggctggaagagttcctgatgtactgggagcagatgctggaccccaccaagcctccatctcttgcacctggcggaggcggaggatctggtggcggaggaagcggtggcggcggatctacagaaaagcggctgctggaagaagccgagagagcccacagagagcagaaagagatcatcaagaaggcccaagagctgcaccgcagactggaagagattgtgcggcagagcggcagctctgaggaagccaagaaagaggccaagaagatcctcgaagagatccgcgagctgagcaagcggagtctggaactgctgagagagatcctgtacctgagccaagaacagaaaggcagcctggtgcctagaggcggcggtggaagtggcggtggtggatcaggcggcggaggctctgagagaaagtgctgcgttgagtgccctccttgtcctgactacaaggacgacgacgacaagcaccaccaccatcaccactga

SEQ ID NO: 25 - DNA, 1121 bp, Artificial Sequence
  Synthetic polynucleotide
gccaccatgtacagaatgcagctgctgagctgtatcgccctgtctctggccctggtcaccaacagcgatatcaagctgcagcagtctggcgccgaactggctagacctggcgcctctgtgaagatgagctgcaagaccagcggctacaccttcaccagatacaccatgcactgggtcaagcagaggccaggccaaggactgggactcgagtggatcggctacatcaaccccsagcagaggctacaccaactacaaccagaagttcaaggacaaggccacactgacctaccgacaagagcagcagcacagcctacatgcagctgagcagcctgaccagcgaagatagcgccgtgtactactgcgcccggtactacgacgatcactactgcctggattactggggccagggcacaaccctgacagtgtcatctgtggaaggcggctctggtggcagcggaggttctggtggatctggcggagtggacgacatccagctgacacagagccctgccatcatgtctgctagccctggcggcaaagtgaccatgacctgtagagccagcagcagcgtgtcctacatgaactggtatcagcagaagtccggcacaagccccaagcggtggatctacgatacaagcaaggtggccagcggcgtgccctacagattttctggctctggcagcggcaccagctactccctgacaatcagcagcatggaagccgaggatgccgccacctactactgccagcagtggtccagcaatcccctgacatttggagccggcaccaagctggaactgaaaggcggcggaggaagcggaggcggaggatccggtggtggcggatctacagagaagagactgctggaagaggccgagagagcccacagagagcagaaagagatcatcaagaaggcccaagagctgcaccgcagactggaagagattgtgcggcagagcggcagctctgaggaagccaagaaagaggccaagaagatcctcgaagagatccgcgagctgagcaagcggagtctggaactgctgagagagatcctgtacctgagccaagaacagaaaggcagcctggtgcctcgggactacaaggacgacgacgacaagtga

SEQ ID NO: 26 - protein, 388 aa, Artificial Sequence
  Synthetic polypeptide
MYRMQLLSCIALSLALVTNSEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGGSGGGGSGGGGSTEKRLLEEAERAHREQKEIIKKAQELHRRLEEIVRQSGSSEEAKKEAKKILEEIRELSKRSLELLREILYLSQEQKGSLVPRERKCCVECPPCPDYKDDDDKHHHHHH

SEQ ID NO: 27 - protein, 603 aa, Artificial Sequence
  Synthetic polypeptide
MYRMQLLSCIALSLALVTNSEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGGSGGGGSGGGGSTEKRLLEEAERAHREQKEIIKKAQELHRRLEEIVRQSGSSEEAKKEAKKILEEIRELSKRSLELLREILYLSQEQKGSLVPRERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDYKDDDKHHHHHH

SEQ ID NO: 28 - protein, 117 aa, Artificial Sequence
  Synthetic polypeptide
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 29 - protein, 108 aa, Artificial Sequence
  Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 30 - protein, 20 aa, Artificial Sequence
  Synthetic polypeptide
GGGGSGGGGSGGGGSGGGGS